SEQUENCE LISTING (1144 sequences)

SEQ ID NO: 1 - protein, 10 aa, Arabidopsis Thaliana
  Sequence located in GRF1. at 61-70 and may interact with
IGARRASWRI

SEQ ID NO: 2 - protein, 10 aa, Arabidopsis Thaliana
  Sequence located in ATPK1 OR ATPK6. at 430-439 and may
DSPASSPSSD

SEQ ID NO: 3 - protein, 10 aa, Arabidopsis Thaliana
  Sequence located in GRF1. at 162-171 and may interact with
AAQDIANSEL

SEQ ID NO: 4 - protein, 10 aa, Arabidopsis Thaliana
  Sequence located in AHA1. at 837-846 and may interact with
KFAIRYILSG

SEQ ID NO: 5 - protein, 10 aa, Arabidopsis Thaliana
  Sequence located in GRF4. a 2-11 and may interact with
AAPPASSSAR

SEQ ID NO: 6 - protein, 10 aa, Arabidopsis Thaliana
  Sequence located in GRP7. a 196-205 and may interact with
AGGGAGGGGG

SEQ ID NO: 7 - protein, 10 aa, Arabidopsis Thaliana
  Sequence located in GRF4. a 2-11 and may interact with
AAPPASSSAR

SEQ ID NO: 8 - protein, 10 aa, Arabidopsis Thaliana
  Sequence located in GRP7. a 138-147 and may interact with
AGGGAGGGGG

SEQ ID NO: 9 - protein, 10 aa, Arabidopsis Thaliana
  Sequence located in GRF4. a 62-71 and may interact with
IGARRASWRI

SEQ ID NO: 10 - protein, 10 aa, Arabidopsis Thaliana
  Sequence located in ATPK1 O ATPK6. at 430-439 and may
DSPASSPSSD

SEQ ID NO: 11 - protein, 10 aa, Arabidopsis Thaliana
  Sequence located in AFT1. a 3-12 and may interact with
ATLGRDQYVY

SEQ ID NO: 12 - protein, 10 aa, Arabidopsis Thaliana
  Sequence located in Unknown at 140-149 and may interact with
SGKSSVLIDI

SEQ ID NO: 13 - protein, 10 aa, Arabidopsis Thaliana
  Sequence located in GRF2 OR GF14. at 56-65 and may interact
IGARRASWRI

SEQ ID NO: 14 - protein, 10 aa, Arabidopsis Thaliana
  Sequence located in ATPK1 O ATPK6. at 430-439 and may
DSPASSPSSD

SEQ ID NO: 15 - protein, 10 aa, Arabidopsis Thaliana
  Sequence located in Unknown at 55-64 and may interact with
IGARRASWRI

SEQ ID NO: 16 - protein, 10 aa, Arabidopsis Thaliana
  Sequence located in ATPK1 O ATPK6. at 430-439 and may
DSPASSPSSD

SEQ ID NO: 17 - protein, 10 aa, Arabidopsis Thaliana
  Sequence located in RCI2. a 3-12 and may interact with
ATLGRDQYVY

SEQ ID NO: 18 - protein, 10 aa, Arabidopsis Thaliana
  Sequence located in Unknown at 140-149 and may interact with
SGKSSVLIDI

SEQ ID NO: 19 - protein, 10 aa, Arabidopsis Thaliana
  Sequence located in GRF5. a 58-67 and may interact with
IGARRASWRI

SEQ ID NO: 20 - protein, 10 aa, Arabidopsis Thaliana
  Sequence located in ATPK1 O ATPK6. at 430-439 and may
DSPASSPSSD

SEQ ID NO: 21 - protein, 10 aa, Arabidopsis Thaliana
  Sequence located in Unknown at 67-76 and may interact with
CNRLRAATTL

SEQ ID NO: 22 - protein, 10 aa, Arabidopsis Thaliana
  Sequence located in BIOB OR BIO2 OR T01O24.10. at 94-103
ECCSRPKTVG

SEQ ID NO: 23 - protein, 10 aa, Arabidopsis Thaliana
  Sequence located in Unknown at 67-76 and may interact with
CNRLRAATTL

SEQ ID NO: 24 - protein, 10 aa, Arabidopsis Thaliana
  Sequence located in CYP71B7 at 95-104 and may interact with
ECCSRPETVA

SEQ ID NO: 25 - protein, 10 aa, Arabidopsis Thaliana
  Sequence located in Unknown at 70-79 and may interact with
LRAATTLTSL

SEQ ID NO: 26 - protein, 10 aa, Arabidopsis Thaliana
  Sequence located in Unknown at 499-508 and may interact with
EARERSRSAQ

SEQ ID NO: 27 - protein, 10 aa, Arabidopsis Thaliana
  Sequence located in ASP1 OR F7F1.18. at 63-72 and may interact
GVGAYRDDNG

SEQ ID NO: 28 - protein, 10 aa, Arabidopsis Thaliana
  Sequence located in P5CSA O P5CS1 OR T5I7.10. at 250-259
AYAGIPVIIT

SEQ ID NO: 29 - protein, 10 aa, Arabidopsis Thaliana
  Sequence located in ASP1 OR F7F1.18. at 19-28 and may interact
ISGHFGGLRS

SEQ ID NO: 30 - protein, 10 aa, Arabidopsis Thaliana
  Sequence located in RPB205 R RPII OR RPB1. at 866-875 and
DTAVKTSETG

SEQ ID NO: 31 - protein, 10 aa, Arabidopsis Thaliana
  Sequence located in ASP1 OR F7F1.18. at 19-28 and may interact
ISGHFGGLRS

SEQ ID NO: 32 - protein, 10 aa, Arabidopsis Thaliana
  Sequence located in RPB205 R RPII OR RPB1. at 840-849 and
DTAVKTSETG

SEQ ID NO: 33 - protein, 10 aa, Arabidopsis Thaliana
  Sequence located in ASP4. a 279-288 and may interact with
SVRLFAERGM

SEQ ID NO: 34 - protein, 10 aa, Arabidopsis Thaliana
  Sequence located in AK1. at 391-400 and may interact with
RDPKKRLTAH

SEQ ID NO: 35 - protein, 10 aa, Arabidopsis Thaliana
  Sequence located in ASP4. a 275-284 and may interact with
EDAASVRLFA

SEQ ID NO: 36 - protein, 10 aa, Arabidopsis Thaliana
  Sequence located in CHLI OR CCSA. at 187-196 and may
LVGSGNPEEG

SEQ ID NO: 37 - protein, 10 aa, Arabidopsis Thaliana
  Sequence located in ASP4. a 274-283 and may interact with
DEDAASVRLF

SEQ ID NO: 38 - protein, 10 aa, Arabidopsis Thaliana
  Sequence located in CHLI OR CCSA. at 186-195 and may
ILVGSGNPEE

SEQ ID NO: 39 - protein, 10 aa, Arabidopsis Thaliana
  Sequence located in ACO OR AT1. at 269-278 and may interact
KEAEKEKKEN

SEQ ID NO: 40 - protein, 10 aa, Arabidopsis Thaliana
  Sequence located in CYP71B3 at 4-13 and may interact with
LLSFFLLLLV

SEQ ID NO: 41 - protein, 10 aa, Arabidopsis Thaliana
  Sequence located in ACO OR AT1. at 268-277 and may interact
GKEAEKEKKE

SEQ ID NO: 42 - protein, 10 aa, Arabidopsis Thaliana
  Sequence located in CYP71B3 at 3-12 and may interact with
SLLSFFLLLL

SEQ ID NO: 43 - protein, 10 aa, Arabidopsis Thaliana
  Sequence located in Unknown at 45-54 and may interact with
IPSRRLSVSC

SEQ ID NO: 44 - protein, 10 aa, Arabidopsis Thaliana
  Sequence located in AG. at 1-60 and may interact with Sequence
TGNGKSATGN

SEQ ID NO: 45 - protein, 10 aa, Arabidopsis Thaliana
  Sequence located in Unknown at 46-55 and may interact with
PSRRLSVSCA

SEQ ID NO: 46 - protein, 10 aa, Arabidopsis Thaliana
  Sequence located in AG. at 0-59 and may interact with Sequence
RTGNGKSATG

SEQ ID NO: 47 - protein, 10 aa, Arabidopsis Thaliana
  Sequence located in Unknown at 47-56 and may interact with
SRRLSVSCAA

SEQ ID NO: 48 - protein, 10 aa, Arabidopsis Thaliana
  Sequence located in AG. at 9-58 and may interact with Sequence
GRTGNGKSAT

SEQ ID NO: 49 - protein, 10 aa, Arabidopsis Thaliana
  Sequence located in ACT1 OR AAC1. at 293-302 and may
KDLYGNIVLS

SEQ ID NO: 50 - protein, 10 aa, Arabidopsis Thaliana
  Sequence located in CAD1. a 36-45 and may interact with
GENDVTVKIL

SEQ ID NO: 51 - protein, 10 aa, Arabidopsis Thaliana
  Sequence located in ACT1 OR AAC1. at 142-151 and may
LSLYASGRTT

SEQ ID NO: 52 - protein, 10 aa, Arabidopsis Thaliana
  Sequence located in RPL12-B at 114-123 and may interact with
GGATASVERQ

SEQ ID NO: 53 - protein, 10 aa, Arabidopsis Thaliana
  Sequence located in ACT2 AN ACT7. at 293-302 and may
KDLYGNIVLS

SEQ ID NO: 54 - protein, 10 aa, Arabidopsis Thaliana
  Sequence located in CAD1. a 36-45 and may interact with
GENDVTVKIL

SEQ ID NO: 55 - protein, 10 aa, Arabidopsis Thaliana
  Sequence located in ACT2 AN ACT7. at 142-151 and may
LSLYASGRTT

SEQ ID NO: 56 - protein, 10 aa, Arabidopsis Thaliana
  Sequence located in RPL12-B at 114-123 and may interact with
GGATASVERQ

SEQ ID NO: 57 - protein, 10 aa, Arabidopsis Thaliana
  Sequence located in ACT3 OR F13M22.12. at 293-302 and may
KDLYGNIVLS

SEQ ID NO: 58 - protein, 10 aa, Arabidopsis Thaliana
  Sequence located in CAD1. a 36-45 and may interact with
GENDVTVKIL

SEQ ID NO: 59 - protein, 10 aa, Arabidopsis Thaliana
  Sequence located in ACT3 OR F13M22.12. at 142-151 and may
LSLYASGRTT

SEQ ID NO: 60 - protein, 10 aa, Arabidopsis Thaliana
  Sequence located in RPL12-B at 114-123 and may interact with
GGATASVERQ

SEQ ID NO: 61 - protein, 10 aa, Arabidopsis Thaliana
  Sequence located in ACT4. a 293-302 and may interact with
KDLYGNIVLS

SEQ ID NO: 62 - protein, 10 aa, Arabidopsis Thaliana
  Sequence located in CAD1. a 36-45 and may interact with
GENDVTVKIL

SEQ ID NO: 63 - protein, 10 aa, Arabidopsis Thaliana
  Sequence located in ACT4. a 142-151 and may interact with
LSLYASGRTT

SEQ ID NO: 64 - protein, 10 aa, Arabidopsis Thaliana
  Sequence located in RPL12-B at 114-123 and may interact with
GGATASVERQ

SEQ ID NO: 65 - protein, 10 aa, Arabidopsis Thaliana
  Sequence located in ACT11. t 293-302 and may interact with
KDLYGNIVLS

SEQ ID NO: 66 - protein, 10 aa, Arabidopsis Thaliana
  Sequence located in CAD1. a 36-45 and may interact with
GENDVTVKIL

SEQ ID NO: 67 - protein, 10 aa, Arabidopsis Thaliana
  Sequence located in ACT11. t 142-151 and may interact with
LSLYASGRTT

SEQ ID NO: 68 - protein, 10 aa, Arabidopsis Thaliana
  Sequence located in RPL12-B at 114-123 and may interact with
GGATASVERQ

SEQ ID NO: 69 - protein, 10 aa, Arabidopsis Thaliana
  Sequence located in ACT12. t 293-302 and may interact with
KDLYGNIVLS

SEQ ID NO: 70 - protein, 10 aa, Arabidopsis Thaliana
  Sequence located in CAD1. a 36-45 and may interact with
GENDVTVKIL

SEQ ID NO: 71 - protein, 10 aa, Arabidopsis Thaliana
  Sequence located in ACT12. t 142-151 and may interact with
LSLYASGRTT

SEQ ID NO: 72 - protein, 10 aa, Arabidopsis Thaliana
  Sequence located in RPL12-B at 114-123 and may interact with
GGATASVERQ

SEQ ID NO: 73 - protein, 10 aa, Arabidopsis Thaliana
  Sequence located in ADH1 OR ADH OR F22K20.19. at 169-178
LDKVCIVSCG

SEQ ID NO: 74 - protein, 10 aa, Arabidopsis Thaliana
  Sequence located in A6. at 99-308 and may interact with
AATYNRNLIK

SEQ ID NO: 75 - protein, 10 aa, Arabidopsis Thaliana
  Sequence located in ADH1 OR ADH OR F22K20.19. at 239-248
GVTECVNPKD

SEQ ID NO: 76 - protein, 10 aa, Arabidopsis Thaliana
  Sequence located in HSP101. at 230-239 and may interact with
PNSLTDVRLI

SEQ ID NO: 77 - protein, 10 aa, Arabidopsis Thaliana
  Sequence located in ADH2 OR ADHIII. at 238-247 and may
FGVNEFVNPK

SEQ ID NO: 78 - protein, 10 aa, Arabidopsis Thaliana
  Sequence located in ADH2 OR ADHIII. at 238-247 and may
FGVNEFVNPK

SEQ ID NO: 79 - protein, 10 aa, Arabidopsis Thaliana
  Sequence located in ADH2 OR ADHIII. at 212-221 and may
EGAKTAGASR

SEQ ID NO: 80 - protein, 10 aa, Arabidopsis Thaliana
  Sequence located in GAPA. a 15-24 and may interact with
FSGLRSSSAS

SEQ ID NO: 81 - protein, 10 aa, Arabidopsis Thaliana
  Sequence located in ADH2 OR ADHIII. at 364-373 and may
LLHEGTCLRC

SEQ ID NO: 82 - protein, 10 aa, Arabidopsis Thaliana
  Sequence located in ABI2. a 93-102 and may interact with
KKVLSRTESR

SEQ ID NO: 83 - protein, 10 aa, Arabidopsis Thaliana
  Sequence located in ADH2 OR ADHIII. at 101-110 and may
ECKFCKSGKT

SEQ ID NO: 84 - protein, 10 aa, Arabidopsis Thaliana
  Sequence located in RBCL. a 125-134 and may interact with
FGFKALAALR

SEQ ID NO: 85 - protein, 10 aa, Arabidopsis Thaliana
  Sequence located in ADH2 OR ADHIII. at 208-217 and may
LAVAEGAKTA

SEQ ID NO: 86 - protein, 10 aa, Arabidopsis Thaliana
  Sequence located in RPOMT. t 57-66 and may interact with
GGLSSFRHGQ

SEQ ID NO: 87 - protein, 10 aa, Arabidopsis Thaliana
  Sequence located in ANT2. a 27-36 and may interact with
IRGYASASKR

SEQ ID NO: 88 - protein, 10 aa, Arabidopsis Thaliana
  Sequence located in COB OR YTB. at 83-92 and may interact
PLTGGGIPSY

SEQ ID NO: 89 - protein, 10 aa, Arabidopsis Thaliana
  Sequence located in ANT2. a 338-347 and may interact with
IVKKEGAKSL

SEQ ID NO: 90 - protein, 10 aa, Arabidopsis Thaliana
  Sequence located in TMKL1. t 509-518 and may interact with
DDFFFARLTE

SEQ ID NO: 91 - protein, 10 aa, Arabidopsis Thaliana
  Sequence located in AFC1. a 3-12 and may interact with
SSVYRDKASS

SEQ ID NO: 92 - protein, 10 aa, Arabidopsis Thaliana
  Sequence located in F7G19.2 at 70-79 and may interact with
RRYVSIFSAA

SEQ ID NO: 93 - protein, 10 aa, Arabidopsis Thaliana
  Sequence located in AGL6 OR F17K2.18. at 108-117 and may
RNLLGEDLGE

SEQ ID NO: 94 - protein, 10 aa, Arabidopsis Thaliana
  Sequence located in PPH1. a 129-138 and may interact with
AIKEALIKAF

SEQ ID NO: 95 - protein, 10 aa, Arabidopsis Thaliana
  Sequence located in AGL8. a 153-162 and may interact with
ALQKKDKALQ

SEQ ID NO: 96 - protein, 10 aa, Arabidopsis Thaliana
  Sequence located in APG. at 432-441 and may interact with
SKLLLILGQL

SEQ ID NO: 97 - protein, 10 aa, Arabidopsis Thaliana
  Sequence located in AG. at 55-264 and may interact with
KKEQEELESK

SEQ ID NO: 98 - protein, 10 aa, Arabidopsis Thaliana
  Sequence located in Unknown at 7-16 and may interact with
LTFQLLLLLL

SEQ ID NO: 99 - protein, 10 aa, Arabidopsis Thaliana
  Sequence located in AG. at 55-264 and may interact with
KKEQEELESK

SEQ ID NO: 100 - protein, 10 aa, Arabidopsis Thaliana
  Sequence located in ILL2. a 9-18 and may interact with
LTFQLLLFLL

SEQ ID NO: 101 - protein, 10 aa, Arabidopsis Thaliana
  Sequence located in AG. at 56-265 and may interact with
KEQEELESKG

SEQ ID NO: 102 - protein, 10 aa, Arabidopsis Thaliana
  Sequence located in ILL2. a 8-17 and may interact with
SLTFQLLLFL

SEQ ID NO: 103 - protein, 10 aa, Arabidopsis Thaliana
  Sequence located in AG. at 9-78 and may interact with Sequence
KSKTKSSGVT

SEQ ID NO: 104 - protein, 10 aa, Arabidopsis Thaliana
  Sequence located in NIT1. a 70-79 and may interact with
GYPRGFRFGL

SEQ ID NO: 105 - protein, 10 aa, Arabidopsis Thaliana
  Sequence located in AG. at 6-75 and may interact with Sequence
KVFKSKTKSS

SEQ ID NO: 106 - protein, 10 aa, Arabidopsis Thaliana
  Sequence located in TYKY OR YUP8H12R.37. at 78-87 and may
RGLSLTLKYF

SEQ ID NO: 107 - protein, 10 aa, Arabidopsis Thaliana
  Sequence located in AMT1. a 285-294 and may interact with
LVGKGLTFDS

SEQ ID NO: 108 - protein, 10 aa, Arabidopsis Thaliana
  Sequence located in Unknown at 175-184 and may interact with
QYSLPKGEVG

SEQ ID NO: 109 - protein, 10 aa, Arabidopsis Thaliana
  Sequence located in AMT1. a 39-48 and may interact with
VTEKDLAKDG

SEQ ID NO: 110 - protein, 10 aa, Arabidopsis Thaliana
  Sequence located in CYP83A1 OR CYP83. at 210-219 and may
SVLGKIFFSD

SEQ ID NO: 111 - protein, 10 aa, Arabidopsis Thaliana
  Sequence located in AMT1. a 134-143 and may interact with
VLASSVSDES

SEQ ID NO: 112 - protein, 10 aa, Arabidopsis Thaliana
  Sequence located in T29E15. 3. at 375-384 and may interact with
TLIADAASKD

SEQ ID NO: 113 - protein, 10 aa, Arabidopsis Thaliana
  Sequence located in AMT1. a 61-70 and may interact with
DAHLSGLLAQ

SEQ ID NO: 114 - protein, 10 aa, Arabidopsis Thaliana
  Sequence located in Unknown at 617-626 and may interact with
LSKETGEVSV

SEQ ID NO: 115 - protein, 10 aa, Arabidopsis Thaliana
  Sequence located in AMT1. a 62-71 and may interact with
AHLSGLLAQV

SEQ ID NO: 116 - protein, 10 aa, Arabidopsis Thaliana
  Sequence located in Unknown at 616-625 and may interact with
YLSKETGEVS

SEQ ID NO: 117 - protein, 10 aa, Arabidopsis Thaliana
  Sequence located in AMT1. a 63-72 and may interact with
HLSGLLAQVS

SEQ ID NO: 118 - protein, 10 aa, Arabidopsis Thaliana
  Sequence located in Unknown at 615-624 and may interact with
GYLSKETGEV

SEQ ID NO: 119 - protein, 10 aa, Arabidopsis Thaliana
  Sequence located in Unknown at 290-299 and may interact with
TLAGCTAALT

SEQ ID NO: 120 - protein, 10 aa, Arabidopsis Thaliana
  Sequence located in GBF3 OR T3F17.8. at 198-207 and may
RESAARSRER

SEQ ID NO: 121 - protein, 10 aa, Arabidopsis Thaliana
  Sequence located in Unknown at 292-301 and may interact with
AGCTAALTTL

SEQ ID NO: 122 - protein, 10 aa, Arabidopsis Thaliana
  Sequence located in Unknown at 8-17 and may interact with
QSSEGSRRSR

SEQ ID NO: 123 - protein, 10 aa, Arabidopsis Thaliana
  Sequence located in Unknown at 86-95 and may interact with
AGGLFYYLFG

SEQ ID NO: 124 - protein, 10 aa, Arabidopsis Thaliana
  Sequence located in PROC1. t 63-72 and may interact with
STSEEVVKES

SEQ ID NO: 125 - protein, 10 aa, Arabidopsis Thaliana
  Sequence located in ATHB-7. at 56-65 and may interact with
LRDRIDSVKN

SEQ ID NO: 126 - protein, 10 aa, Arabidopsis Thaliana
  Sequence located in DGK1. a 264-273 and may interact with
VFNAVDSISK

SEQ ID NO: 127 - protein, 10 aa, Arabidopsis Thaliana
  Sequence located in ATHB-7. at 57-66 and may interact with
RDRIDSVKNT

SEQ ID NO: 128 - protein, 10 aa, Arabidopsis Thaliana
  Sequence located in DGK1. a 263-272 and may interact with
SVFNAVDSIS

SEQ ID NO: 129 - protein, 10 aa, Arabidopsis Thaliana
  Sequence located in ATHB-7. at 38-47 and may interact with
SPPSRASSVS

SEQ ID NO: 130 - protein, 10 aa, Arabidopsis Thaliana
  Sequence located in PME2. a 532-541 and may interact with
RGGGAGTANR

SEQ ID NO: 131 - protein, 10 aa, Arabidopsis Thaliana
  Sequence located in ATHB-7. at 190-199 and may interact with
EAGTLPTAKE

SEQ ID NO: 132 - protein, 10 aa, Arabidopsis Thaliana
  Sequence located in ASA2 OR T27A16.21. at 129-138 and may
LLSGGQGTRL

SEQ ID NO: 133 - protein, 10 aa, Arabidopsis Thaliana
  Sequence located in Unknown at 549-558 and may interact with
IGSAALVSLA

SEQ ID NO: 134 - protein, 10 aa, Arabidopsis Thaliana
  Sequence located in GBF1. a 304-313 and may interact with
NAAGSKDGEG

SEQ ID NO: 135 - protein, 10 aa, Arabidopsis Thaliana
  Sequence located in Unknown at 551-560 and may interact with
SAALVSLALF

SEQ ID NO: 136 - protein, 10 aa, Arabidopsis Thaliana
  Sequence located in T06B20. 4 OR T09D09.20. at 406-415 and
EEGKAYQSGG

SEQ ID NO: 137 - protein, 10 aa, Arabidopsis Thaliana
  Sequence located in Unknown at 132-141 and may interact with
TTRTCKPALA

SEQ ID NO: 138 - protein, 10 aa, Arabidopsis Thaliana
  Sequence located in Unknown at 367-376 and may interact with
GRSGRFGRKG

SEQ ID NO: 139 - protein, 10 aa, Arabidopsis Thaliana
  Sequence located in Unknown at 131-140 and may interact with
DTTRTCKPAL

SEQ ID NO: 140 - protein, 10 aa, Arabidopsis Thaliana
  Sequence located in Unknown at 366-375 and may interact with
IGRSGRFGRK

SEQ ID NO: 141 - protein, 10 aa, Arabidopsis Thaliana
  Sequence located in Unknown at 132-141 and may interact with
TTRTCKPALA

SEQ ID NO: 142 - protein, 10 aa, Arabidopsis Thaliana
  Sequence located in TIF4A-2 at 367-376 and may interact with
GRSGRFGRKG

SEQ ID NO: 143 - protein, 10 aa, Arabidopsis Thaliana
  Sequence located in Unknown at 131-140 and may interact with
DTTRTCKPAL

SEQ ID NO: 144 - protein, 10 aa, Arabidopsis Thaliana
  Sequence located in TIF4A-2 at 366-375 and may interact with
IGRSGRFGRK

SEQ ID NO: 145 - protein, 10 aa, Arabidopsis Thaliana
  Sequence located in Unknown at 439-448 and may interact with
DVADSCRTGA

SEQ ID NO: 146 - protein, 10 aa, Arabidopsis Thaliana
  Sequence located in RPS13. t 68-77 and may interact with
IHGIGRTRAR

SEQ ID NO: 147 - protein, 10 aa, Arabidopsis Thaliana
  Sequence located in AOX1B. t 167-176 and may interact with
LVHCKSLRRF

SEQ ID NO: 148 - protein, 10 aa, Arabidopsis Thaliana
  Sequence located in CYP71B9 OR T8K22.12. at 149-158 and
EATKALRMNK

SEQ ID NO: 149 - protein, 10 aa, Arabidopsis Thaliana
  Sequence located in IAA2. a 38-47 and may interact with
SPSASASLRS

SEQ ID NO: 150 - protein, 10 aa, Arabidopsis Thaliana
  Sequence located in Unknown at 63-72 and may interact with
GAKGGRCRWG

SEQ ID NO: 151 - protein, 10 aa, Arabidopsis Thaliana
  Sequence located in IAA2. a 50-59 and may interact with
ARRSSLSSTS

SEQ ID NO: 152 - protein, 10 aa, Arabidopsis Thaliana
  Sequence located in PHYC. a 9-18 and may interact with
TSGGEAASSG

SEQ ID NO: 153 - protein, 10 aa, Arabidopsis Thaliana
  Sequence located in IAA2. a 22-31 and may interact with
KSSLSSPSVS

SEQ ID NO: 154 - protein, 10 aa, Arabidopsis Thaliana
  Sequence located in RAN-1. t 20-29 and may interact with
GDGGTGKTTF

SEQ ID NO: 155 - protein, 10 aa, Arabidopsis Thaliana
  Sequence located in IAA2. a 35-44 and may interact with
TLSSPSASAS

SEQ ID NO: 156 - protein, 10 aa, Arabidopsis Thaliana
  Sequence located in RPL23. t 2-11 and may interact with
SKRGRGGTSG

SEQ ID NO: 157 - protein, 10 aa, Arabidopsis Thaliana
  Sequence located in IAA2. a 5-14 and may interact with
ASSTTLISSP

SEQ ID NO: 158 - protein, 10 aa, Arabidopsis Thaliana
  Sequence located in Unknown at 76-85 and may interact with
RGGDKRSGGG

SEQ ID NO: 159 - protein, 10 aa, Arabidopsis Thaliana
  Sequence located in CAC1. a 7-16 and may interact with
SVTSPAAAAS

SEQ ID NO: 160 - protein, 10 aa, Arabidopsis Thaliana
  Sequence located in CTR1. a 149-158 and may interact with
ADSAGGSSSG

SEQ ID NO: 161 - protein, 10 aa, Arabidopsis Thaliana
  Sequence located in CAC1. a 180-189 and may interact with
AAPAPSTPAS

SEQ ID NO: 162 - protein, 10 aa, Arabidopsis Thaliana
  Sequence located in CYSJ. a 337-346 and may interact with
RGWSRASGRT

SEQ ID NO: 163 - protein, 10 aa, Arabidopsis Thaliana
  Sequence located in CAC1. a 7-16 and may interact with
SVTSPAAAAS

SEQ ID NO: 164 - protein, 10 aa, Arabidopsis Thaliana
  Sequence located in RAB18. t 35-44 and may interact with
GYGTGGGGGA

SEQ ID NO: 165 - protein, 10 aa, Arabidopsis Thaliana
  Sequence located in CAC1. a 180-189 and may interact with
AAPAPSTPAS

SEQ ID NO: 166 - protein, 10 aa, Arabidopsis Thaliana
  Sequence located in GRP7. a 201-210 and may interact with
GGGGGAGGGG

SEQ ID NO: 167 - protein, 10 aa, Arabidopsis Thaliana
  Sequence located in CAC1. a 7-16 and may interact with
SVTSPAAAAS

SEQ ID NO: 168 - protein, 10 aa, Arabidopsis Thaliana
  Sequence located in HAT9. a 231-240 and may interact with
GNGGGGGGSG

SEQ ID NO: 169 - protein, 10 aa, Arabidopsis Thaliana
  Sequence located in CAC1. a 50-59 and may interact with
SKPSRSSYPV

SEQ ID NO: 170 - protein, 10 aa, Arabidopsis Thaliana
  Sequence located in Unknown at 364-373 and may interact with
HRIGRSGRFG

SEQ ID NO: 171 - protein, 10 aa, Arabidopsis Thaliana
  Sequence located in CAC1. a 195-204 and may interact with
PPTPAKSSLP

SEQ ID NO: 172 - protein, 10 aa, Arabidopsis Thaliana
  Sequence located in Unknown at 367-376 and may interact with
GRSGRFGRKG

SEQ ID NO: 173 - protein, 10 aa, Arabidopsis Thaliana
  Sequence located in CAC1. a 50-59 and may interact with
SKPSRSSYPV

SEQ ID NO: 174 - protein, 10 aa, Arabidopsis Thaliana
  Sequence located in TIF4A-2 at 364-373 and may interact with
HRIGRSGRFG

SEQ ID NO: 175 - protein, 10 aa, Arabidopsis Thaliana
  Sequence located in CAC1. a 195-204 and may interact with
PPTPAKSSLP

SEQ ID NO: 176 - protein, 10 aa, Arabidopsis Thaliana
  Sequence located in TIF4A-2 at 367-376 and may interact with
GRSGRFGRKG

SEQ ID NO: 177 - protein, 10 aa, Arabidopsis Thaliana
  Sequence located in CAC1. a 90-99 and may interact with
NSLKESSASS

SEQ ID NO: 178 - protein, 10 aa, Arabidopsis Thaliana
  Sequence located in Unknown at 457-466 and may interact with
IAQLFRASRA

SEQ ID NO: 179 - protein, 10 aa, Arabidopsis Thaliana
  Sequence located in CAC1. a 175-184 and may interact with
AAPAAAAPAP

SEQ ID NO: 180 - protein, 10 aa, Arabidopsis Thaliana
  Sequence located in Unknown at 81-90 and may interact with
RSGGGSRRSS

SEQ ID NO: 181 - protein, 10 aa, Arabidopsis Thaliana
  Sequence located in CAC1. a 172-181 and may interact with
APPAAPAAAA

SEQ ID NO: 182 - protein, 10 aa, Arabidopsis Thaliana
  Sequence located in Unknown at 83-92 and may interact with
GGGSRRSSSS

SEQ ID NO: 183 - protein, 10 aa, Arabidopsis Thaliana
  Sequence located in CAC1. a 172-181 and may interact with
APPAAPAAAA

SEQ ID NO: 184 - protein, 10 aa, Arabidopsis Thaliana
  Sequence located in Unknown at 82-91 and may interact with
SGGGSRRSSS

SEQ ID NO: 185 - protein, 10 aa, Arabidopsis Thaliana
  Sequence located in CAC1. a 178-187 and may interact with
AAAAPAPSTP

SEQ ID NO: 186 - protein, 10 aa, Arabidopsis Thaliana
  Sequence located in Unknown at 80-89 and may interact with
RRRGCWRRSC

SEQ ID NO: 187 - protein, 10 aa, Arabidopsis Thaliana
  Sequence located in CAC1. a 179-188 and may interact with
AAAPAPSTPA

SEQ ID NO: 188 - protein, 10 aa, Arabidopsis Thaliana
  Sequence located in Unknown at 79-88 and may interact with
RRRRGCWRRS

SEQ ID NO: 189 - protein, 10 aa, Arabidopsis Thaliana
  Sequence located in CAC1. a 180-189 and may interact with
AAPAPSTPAS

SEQ ID NO: 190 - protein, 10 aa, Arabidopsis Thaliana
  Sequence located in Unknown at 78-87 and may interact with
ARRRRGCWRR

SEQ ID NO: 191 - protein, 10 aa, Arabidopsis Thaliana
  Sequence located in CAC1. a 172-181 and may interact with
APPAAPAAAA

SEQ ID NO: 192 - protein, 10 aa, Arabidopsis Thaliana
  Sequence located in Unknown at 80-89 and may interact with
RRRGCWRRSC

SEQ ID NO: 193 - protein, 10 aa, Arabidopsis Thaliana
  Sequence located in BIOB OR BIO2 OR T01O24.10. at 105-114
GKLSYGFKDI

SEQ ID NO: 194 - protein, 10 aa, Arabidopsis Thaliana
  Sequence located in Unknown at 85-94 and may interact with
TLERITKLVN

SEQ ID NO: 195 - protein, 10 aa, Arabidopsis Thaliana
  Sequence located in BIOB OR BIO2 OR T01O24.10. at 93-102
LECCSRPKTV

SEQ ID NO: 196 - protein, 10 aa, Arabidopsis Thaliana
  Sequence located in FBP. at 400-409 and may interact with
HCLGAAGGLE

SEQ ID NO: 197 - protein, 10 aa, Arabidopsis Thaliana
  Sequence located in CYP71B3 at 72-81 and may interact with
GFAPMVVISS

SEQ ID NO: 198 - protein, 10 aa, Arabidopsis Thaliana
  Sequence located in FBP. at 234-243 and may interact with
AANHNHRGEA

SEQ ID NO: 199 - protein, 10 aa, Arabidopsis Thaliana
  Sequence located in CYP71B3 at 73-82 and may interact with
FAPMVVISSS

SEQ ID NO: 200 - protein, 10 aa, Arabidopsis Thaliana
  Sequence located in FBP. at 233-242 and may interact with
AAANHNHRGE

SEQ ID NO: 201 - protein, 10 aa, Arabidopsis Thaliana
  Sequence located in CYP71B5 at 24-33 and may interact with
LLIFFLLRSP

SEQ ID NO: 202 - protein, 10 aa, Arabidopsis Thaliana
  Sequence located in RPS2 OR T11A07.6. at 21-30 and may
KQDKKKKPRG

SEQ ID NO: 203 - protein, 10 aa, Arabidopsis Thaliana
  Sequence located in CYP71B7 at 94-103 and may interact with
LECCSRPETV

SEQ ID NO: 204 - protein, 10 aa, Arabidopsis Thaliana
  Sequence located in FBP. at 400-409 and may interact with
HCLGAAGGLE

SEQ ID NO: 205 - protein, 10 aa, Arabidopsis Thaliana
  Sequence located in CYP71B7 at 95-104 and may interact with
ECCSRPETVA

SEQ ID NO: 206 - protein, 10 aa, Arabidopsis Thaliana
  Sequence located in FBP. at 399-408 and may interact with
GHCLGAAGGL

SEQ ID NO: 207 - protein, 10 aa, Arabidopsis Thaliana
  Sequence located in CYP71B9 OR T8K22.12. at 309-318 and
FTAGTDTSSS

SEQ ID NO: 208 - protein, 10 aa, Arabidopsis Thaliana
  Sequence located in GSH1. a 512-521 and may interact with
KRRSRVRRAA

SEQ ID NO: 209 - protein, 10 aa, Arabidopsis Thaliana
  Sequence located in CYP71B9 OR T8K22.12. at 24-33 and may
TTTRSGRISR

SEQ ID NO: 210 - protein, 10 aa, Arabidopsis Thaliana
  Sequence located in HEMA1 O HEMA. at 69-78 and may
TANTTTASSS

SEQ ID NO: 211 - protein, 10 aa, Arabidopsis Thaliana
  Sequence located in CYP71B9 OR T8K22.12. at 25-34 and may
TTRSGRISRG

SEQ ID NO: 212 - protein, 10 aa, Arabidopsis Thaliana
  Sequence located in HEMA1 O HEMA. at 68-77 and may
ATANTTTASS

SEQ ID NO: 213 - protein, 10 aa, Arabidopsis Thaliana
  Sequence located in CYP71B9 OR T8K22.12. at 20-29 and may
FLIFTTTRSG

SEQ ID NO: 214 - protein, 10 aa, Arabidopsis Thaliana
  Sequence located in PPH1. a 155-164 and may interact with
EEDESGSTAT

SEQ ID NO: 215 - protein, 10 aa, Arabidopsis Thaliana
  Sequence located in CYP71B9 OR T8K22.12. at 39-48 and may
PPGPPRLPII

SEQ ID NO: 216 - protein, 10 aa, Arabidopsis Thaliana
  Sequence located in RBP31 O RNPT. at 277-286 and may
YDRETGRSRG

SEQ ID NO: 217 - protein, 10 aa, Arabidopsis Thaliana
  Sequence located in CYP76C2 OR F17K2.10. at 39-48 and may
PPGPPRLPII

SEQ ID NO: 218 - protein, 10 aa, Arabidopsis Thaliana
  Sequence located in RBP31 O RNPT. at 277-286 and may
YDRETGRSRG

SEQ ID NO: 219 - protein, 10 aa, Arabidopsis Thaliana
  Sequence located in CYP76C2 OR F17K2.10. at 243-252 and
KTLKACSERL

SEQ ID NO: 220 - protein, 10 aa, Arabidopsis Thaliana
  Sequence located in Unknown at 452-461 and may interact with
LGKLGARLTK

SEQ ID NO: 221 - protein, 10 aa, Arabidopsis Thaliana
  Sequence located in CYP88A3 OR YUP8H12.23. at 402-411 and
IDPEVFPDPR

SEQ ID NO: 222 - protein, 10 aa, Arabidopsis Thaliana
  Sequence located in Unknown at 94-103 and may interact with
PGVWEYLRVN

SEQ ID NO: 223 - protein, 10 aa, Arabidopsis Thaliana
  Sequence located in CYP98A3 OR T20B5.9. at 91-100 and may
LADRHRNRST

SEQ ID NO: 224 - protein, 10 aa, Arabidopsis Thaliana
  Sequence located in COR47 O RD17. at 162-171 and may
STTIPVPVSE

SEQ ID NO: 225 - protein, 10 aa, Arabidopsis Thaliana
  Sequence located in CYP98A3 OR T20B5.9. at 140-149 and may
RPIREDEVTA

SEQ ID NO: 226 - protein, 10 aa, Arabidopsis Thaliana
  Sequence located in Unknown at 195-204 and may interact with
CRDLVLSYGA

SEQ ID NO: 227 - protein, 10 aa, Arabidopsis Thaliana
  Sequence located in CYP98A3 OR T20B5.9. at 101-110 and may
EAFSRNGQDL

SEQ ID NO: 228 - protein, 10 aa, Arabidopsis Thaliana
  Sequence located in PROC1. t 2-11 and may interact with
EILPIPAESF

SEQ ID NO: 229 - protein, 10 aa, Arabidopsis Thaliana
  Sequence located in CYP98A3 OR T20B5.9. at 255-264 and may
EEHTLARQKS

SEQ ID NO: 230 - protein, 10 aa, Arabidopsis Thaliana
  Sequence located in Unknown at 179-188 and may interact with
GLLSGESVFL

SEQ ID NO: 231 - protein, 10 aa, Arabidopsis Thaliana
  Sequence located in CYP98A3 OR T20B5.9. at 256-265 and may
EHTLARQKSS

SEQ ID NO: 232 - protein, 10 aa, Arabidopsis Thaliana
  Sequence located in Unknown at 178-187 and may interact with
GGLLSGESVF

SEQ ID NO: 233 - protein, 10 aa, Arabidopsis Thaliana
  Sequence located in CYP98A3 OR T20B5.9. at 257-266 and may
HTLARQKSSG

SEQ ID NO: 234 - protein, 10 aa, Arabidopsis Thaliana
  Sequence located in Unknown at 177-186 and may interact with
SGGLLSGESV

SEQ ID NO: 235 - protein, 10 aa, Arabidopsis Thaliana
  Sequence located in CAD1. a 296-305 and may interact with
KMVGGSDVGG

SEQ ID NO: 236 - protein, 10 aa, Arabidopsis Thaliana
  Sequence located in HEMC. a 74-83 and may interact with
ASNVRATYHF

SEQ ID NO: 237 - protein, 10 aa, Arabidopsis Thaliana
  Sequence located in CAD1. a 224-233 and may interact with
INHLGADSFL

SEQ ID NO: 238 - protein, 10 aa, Arabidopsis Thaliana
  Sequence located in Unknown at 261-270 and may interact with
EEGISAEVIN

SEQ ID NO: 239 - protein, 10 aa, Arabidopsis Thaliana
  Sequence located in CAM1. a 13-22 and may interact with
GCITTKELGT

SEQ ID NO: 240 - protein, 10 aa, Arabidopsis Thaliana
  Sequence located in NAK. at 20-29 and may interact with
SSKFLSRDGS

SEQ ID NO: 241 - protein, 10 aa, Arabidopsis Thaliana
  Sequence located in (CAM2 O CAL1) AND CAM3 AND
GCITTKELGT

SEQ ID NO: 242 - protein, 10 aa, Arabidopsis Thaliana
  Sequence located in NAK. at 20-29 and may interact with
SSKFLSRDGS

SEQ ID NO: 243 - protein, 10 aa, Arabidopsis Thaliana
  Sequence located in CAS1. a 93-102 and may interact with
VETTLKRGLD

SEQ ID NO: 244 - protein, 10 aa, Arabidopsis Thaliana
  Sequence located in CAS1. a 93-102 and may interact with
VETTLKRGLD

SEQ ID NO: 245 - protein, 10 aa, Arabidopsis Thaliana
  Sequence located in CAS1. a 529-538 and may interact with
LATYELTRSY

SEQ ID NO: 246 - protein, 10 aa, Arabidopsis Thaliana
  Sequence located in Unknown at 124-133 and may interact with
IAACELVGGE

SEQ ID NO: 247 - protein, 10 aa, Arabidopsis Thaliana
  Sequence located in CAS1. a 340-349 and may interact with
NLREKAIRTA

SEQ ID NO: 248 - protein, 10 aa, Arabidopsis Thaliana
  Sequence located in SAM1. a 182-191 and may interact with
GGSDRLLSQV

SEQ ID NO: 249 - protein, 10 aa, Arabidopsis Thaliana
  Sequence located in CAS1. a 341-350 and may interact with
LREKAIRTAI

SEQ ID NO: 250 - protein, 10 aa, Arabidopsis Thaliana
  Sequence located in SAM1. a 181-190 and may interact with
YGGSDRLLSQ

SEQ ID NO: 251 - protein, 10 aa, Arabidopsis Thaliana
  Sequence located in CAS1. a 638-647 and may interact with
VAKACEFLLS

SEQ ID NO: 252 - protein, 10 aa, Arabidopsis Thaliana
  Sequence located in RAN-1. t 71-80 and may interact with
GQEKFGGLRD

SEQ ID NO: 253 - protein, 10 aa, Arabidopsis Thaliana
  Sequence located in CAS1. a 338-347 and may interact with
GANLREKAIR

SEQ ID NO: 254 - protein, 10 aa, Arabidopsis Thaliana
  Sequence located in UBC1. a 107-116 and may interact with
TSIQSLLCDP

SEQ ID NO: 255 - protein, 10 aa, Arabidopsis Thaliana
  Sequence located in CAS1. a 338-347 and may interact with
GANLREKAIR

SEQ ID NO: 256 - protein, 10 aa, Arabidopsis Thaliana
  Sequence located in UBC2 OR T20F6.10. at 107-116 and may
TSIQSLLCDP

SEQ ID NO: 257 - protein, 10 aa, Arabidopsis Thaliana
  Sequence located in CAT1. a 29-38 and may interact with
NSSLTVGTRG

SEQ ID NO: 258 - protein, 10 aa, Arabidopsis Thaliana
  Sequence located in RPS6. a 82-91 and may interact with
SARPYGQRAV

SEQ ID NO: 259 - protein, 10 aa, Arabidopsis Thaliana
  Sequence located in CAT2 OR CAT. at 453-462 and may interact
ALSDPRITHE

SEQ ID NO: 260 - protein, 10 aa, Arabidopsis Thaliana
  Sequence located in F7G19.2 at 374-383 and may interact with
LMGDPGVAKS

SEQ ID NO: 261 - protein, 10 aa, Arabidopsis Thaliana
  Sequence located in Unknown at 23-32 and may interact with
LTGSSGRLNR

SEQ ID NO: 262 - protein, 10 aa, Arabidopsis Thaliana
  Sequence located in COR47 O RD17. at 21-30 and may interact
ESPATTTEVT

SEQ ID NO: 263 - protein, 10 aa, Arabidopsis Thaliana
  Sequence located in Unknown at 22-31 and may interact with
FLTGSSGRLN

SEQ ID NO: 264 - protein, 10 aa, Arabidopsis Thaliana
  Sequence located in COR47 O RD17. at 20-29 and may interact
EESPATTTEV

SEQ ID NO: 265 - protein, 10 aa, Arabidopsis Thaliana
  Sequence located in Unknown at 21-30 and may interact with
RFLTGSSGRL

SEQ ID NO: 266 - protein, 10 aa, Arabidopsis Thaliana
  Sequence located in COR47 O RD17. at 19-28 and may interact
TEESPATTTE

SEQ ID NO: 267 - protein, 10 aa, Arabidopsis Thaliana
  Sequence located in CDC2A O CDC2. at 10-19 and may
LLHFVVFIAS

SEQ ID NO: 268 - protein, 10 aa, Arabidopsis Thaliana
  Sequence located in LTI78 O LTI140 OR RD29B OR COR78.
EEVEHHENGA

SEQ ID NO: 269 - protein, 10 aa, Arabidopsis Thaliana
  Sequence located in CDC2A O CDC2. at 458-467 and may
LKTYEQLSFL

SEQ ID NO: 270 - protein, 10 aa, Arabidopsis Thaliana
  Sequence located in Unknown at 77-86 and may interact with
EETELLVGLK

SEQ ID NO: 271 - protein, 10 aa, Arabidopsis Thaliana
  Sequence located in Unknown at 76-85 and may interact with
PSTKSQSTKS

SEQ ID NO: 272 - protein, 10 aa, Arabidopsis Thaliana
  Sequence located in P5CSA O P5CS1 OR T5I7.10. at 30-39 and
GGRLALGRLG

SEQ ID NO: 273 - protein, 10 aa, Arabidopsis Thaliana
  Sequence located in Unknown at 285-294 and may interact with
NPAERISAKT

SEQ ID NO: 274 - protein, 10 aa, Arabidopsis Thaliana
  Sequence located in RPB205 R RPII OR RPB1. at 49-58 and
VGGLSDTRLG

SEQ ID NO: 275 - protein, 10 aa, Arabidopsis Thaliana
  Sequence located in Unknown at 285-294 and may interact with
NPAERISAKT

SEQ ID NO: 276 - protein, 10 aa, Arabidopsis Thaliana
  Sequence located in RPB205 R RPII OR RPB1. at 49-58 and
VGGLSDTRLG

SEQ ID NO: 277 - protein, 10 aa, Arabidopsis Thaliana
  Sequence located in CDC48. t 590-599 and may interact with
GGGSGGDGGG

SEQ ID NO: 278 - protein, 10 aa, Arabidopsis Thaliana
  Sequence located in FBP. at 44-53 and may interact with
SASASTVSAP

SEQ ID NO: 279 - protein, 10 aa, Arabidopsis Thaliana
  Sequence located in CDC48. t 589-598 and may interact with
RGGGSGGDGG

SEQ ID NO: 280 - protein, 10 aa, Arabidopsis Thaliana
  Sequence located in P5CSB O P5CS2. at 16-25 and may interact
AAVTSAPATS

SEQ ID NO: 281 - protein, 10 aa, Arabidopsis Thaliana
  Sequence located in CDC48. t 590-599 and may interact with
GGGSGGDGGG

SEQ ID NO: 282 - protein, 10 aa, Arabidopsis Thaliana
  Sequence located in P5CSB O P5CS2. at 15-24 and may interact
TAAVTSAPAT

SEQ ID NO: 283 - protein, 10 aa, Arabidopsis Thaliana
  Sequence located in CDC48. t 591-600 and may interact with
GGSGGDGGGA

SEQ ID NO: 284 - protein, 10 aa, Arabidopsis Thaliana
  Sequence located in P5CSB O P5CS2. at 14-23 and may interact
STAAVTSAPA

SEQ ID NO: 285 - protein, 10 aa, Arabidopsis Thaliana
  Sequence located in CDC48. t 592-601 and may interact with
GSGGDGGGAA

SEQ ID NO: 286 - protein, 10 aa, Arabidopsis Thaliana
  Sequence located in P5CSB O P5CS2. at 13-22 and may interact
GSTAAVTSAP

SEQ ID NO: 287 - protein, 10 aa, Arabidopsis Thaliana
  Sequence located in CDC48. t 589-598 and may interact with
RGGGSGGDGG

SEQ ID NO: 288 - protein, 10 aa, Arabidopsis Thaliana
  Sequence located in PAB5. a 27-36 and may interact with
AAVAAAAAAA

SEQ ID NO: 289 - protein, 10 aa, Arabidopsis Thaliana
  Sequence located in CDC48. t 589-598 and may interact with
RGGGSGGDGG

SEQ ID NO: 290 - protein, 10 aa, Arabidopsis Thaliana
  Sequence located in PAB5. a 10-19 and may interact with
PTVAAAAPPP

SEQ ID NO: 291 - protein, 10 aa, Arabidopsis Thaliana
  Sequence located in CDC48. t 258-267 and may interact with
ARAVANETGA

SEQ ID NO: 292 - protein, 10 aa, Arabidopsis Thaliana
  Sequence located in PTR2-A R PTR2. at 55-64 and may interact
SSSFVRNRSR

SEQ ID NO: 293 - protein, 10 aa, Arabidopsis Thaliana
  Sequence located in CDC48. t 590-599 and may interact with
GGGSGGDGGG

SEQ ID NO: 294 - protein, 10 aa, Arabidopsis Thaliana
  Sequence located in Unknown at 100-109 and may interact with
APAAAAVAAP

SEQ ID NO: 295 - protein, 10 aa, Arabidopsis Thaliana
  Sequence located in CDC48. t 590-599 and may interact with
GGGSGGDGGG

SEQ ID NO: 296 - protein, 10 aa, Arabidopsis Thaliana
  Sequence located in RPL12-C at 96-105 and may interact with
APAAAAVAAP

SEQ ID NO: 297 - protein, 10 aa, Arabidopsis Thaliana
  Sequence located in CDC48. t 589-598 and may interact with
RGGGSGGDGG

SEQ ID NO: 298 - protein, 10 aa, Arabidopsis Thaliana
  Sequence located in RPS6. a 260-269 and may interact with
APISAAPAAA

SEQ ID NO: 299 - protein, 10 aa, Arabidopsis Thaliana
  Sequence located in CDC48. t 589-598 and may interact with
RGGGSGGDGG

SEQ ID NO: 300 - protein, 10 aa, Arabidopsis Thaliana
  Sequence located in Unknown at 34-43 and may interact with
PATTGASVSS

SEQ ID NO: 301 - protein, 10 aa, Arabidopsis Thaliana
  Sequence located in CDC48. t 589-598 and may interact with
RGGGSGGDGG

SEQ ID NO: 302 - protein, 10 aa, Arabidopsis Thaliana
  Sequence located in Unknown at 12-21 and may interact with
ASVSSAPSSS

SEQ ID NO: 303 - protein, 10 aa, Arabidopsis Thaliana
  Sequence located in CYCD1 O F20P5.7. at 306-315 and may
TRPSDESSSP

SEQ ID NO: 304 - protein, 10 aa, Arabidopsis Thaliana
  Sequence located in ILR1. a 211-220 and may interact with
RPGTVLAGAG

SEQ ID NO: 305 - protein, 10 aa, Arabidopsis Thaliana
  Sequence located in CYCD1 O F20P5.7. at 298-307 and may
SVRASSTLTR

SEQ ID NO: 306 - protein, 10 aa, Arabidopsis Thaliana
  Sequence located in Unknown at 121-130 and may interact with
GHARRGSQRT

SEQ ID NO: 307 - protein, 10 aa, Arabidopsis Thaliana
  Sequence located in CYCD2. t 245-254 and may interact with
IAAAAAVSVS

SEQ ID NO: 308 - protein, 10 aa, Arabidopsis Thaliana
  Sequence located in F8D20.1 0. at 95-104 and may interact with
NSCSSCHGDA

SEQ ID NO: 309 - protein, 10 aa, Arabidopsis Thaliana
  Sequence located in Unknown at 452-461 and may interact with
AARELEKLPT

SEQ ID NO: 310 - protein, 10 aa, Arabidopsis Thaliana
  Sequence located in PETF. a 89-98 and may interact with
GRKFFELPSS

SEQ ID NO: 311 - protein, 10 aa, Arabidopsis Thaliana
  Sequence located in Unknown at 558-567 and may interact with
DESESGAAGG

SEQ ID NO: 312 - protein, 10 aa, Arabidopsis Thaliana
  Sequence located in F10A8.1 . at 53-62 and may interact with
VLGLGTGSTA

SEQ ID NO: 313 - protein, 10 aa, Arabidopsis Thaliana
  Sequence located in Unknown at 114-123 and may interact with
VAGDGTTCAT

SEQ ID NO: 314 - protein, 10 aa, Arabidopsis Thaliana
  Sequence located in Unknown at 203-212 and may interact with
GSAGCAVSSD

SEQ ID NO: 315 - protein, 10 aa, Arabidopsis Thaliana
  Sequence located in Unknown at 98-107 and may interact with
RVKNVGASLV

SEQ ID NO: 316 - protein, 10 aa, Arabidopsis Thaliana
  Sequence located in A6. at 90-299 and may interact with
DETGANILNA

SEQ ID NO: 317 - protein, 10 aa, Arabidopsis Thaliana
  Sequence located in Unknown at 501-510 and may interact with
GLLLSTLALG

SEQ ID NO: 318 - protein, 10 aa, Arabidopsis Thaliana
  Sequence located in TOP2. a 1249-1258 and may interact with
AKKKASESET

SEQ ID NO: 319 - protein, 10 aa, Arabidopsis Thaliana
  Sequence located in Unknown at 500-509 and may interact with
TGLLLSTLAL

SEQ ID NO: 320 - protein, 10 aa, Arabidopsis Thaliana
  Sequence located in TOP2. a 1248-1257 and may interact with
GAKKKASESE

SEQ ID NO: 321 - protein, 10 aa, Arabidopsis Thaliana
  Sequence located in Unknown at 21-30 and may interact with
PAGILAIGTA

SEQ ID NO: 322 - protein, 10 aa, Arabidopsis Thaliana
  Sequence located in CKB1. a 87-96 and may interact with
SGSDGEDTSW

SEQ ID NO: 323 - protein, 10 aa, Arabidopsis Thaliana
  Sequence located in Unknown at 22-31 and may interact with
AGILAIGTAN

SEQ ID NO: 324 - protein, 10 aa, Arabidopsis Thaliana
  Sequence located in CKB1. a 86-95 and may interact with
VSGSDGEDTS

SEQ ID NO: 325 - protein, 10 aa, Arabidopsis Thaliana
  Sequence located in Unknown at 51-60 and may interact with
SNRRAKVCAS

SEQ ID NO: 326 - protein, 10 aa, Arabidopsis Thaliana
  Sequence located in F7G19.2 at 468-477 and may interact with
GITTSLNART

SEQ ID NO: 327 - protein, 10 aa, Arabidopsis Thaliana
  Sequence located in Unknown at 24-33 and may interact with
KSTSLSSSRS

SEQ ID NO: 328 - protein, 10 aa, Arabidopsis Thaliana
  Sequence located in SUOX. a 22-31 and may interact with
AAAARQRSAL

SEQ ID NO: 329 - protein, 10 aa, Arabidopsis Thaliana
  Sequence located in Unknown at 257-266 and may interact with
LDGPSPPVGA

SEQ ID NO: 330 - protein, 10 aa, Arabidopsis Thaliana
  Sequence located in (AVA-P1 OR T4L20.300) AND AVA-P2
EVPGRRGYPG

SEQ ID NO: 331 - protein, 10 aa, Arabidopsis Thaliana
  Sequence located in CNX2. a 27-36 and may interact with
VGSGSVTRTI

SEQ ID NO: 332 - protein, 10 aa, Arabidopsis Thaliana
  Sequence located in PDH2. a 295-304 and may interact with
DPGSTYRTRD

SEQ ID NO: 333 - protein, 10 aa, Arabidopsis Thaliana
  Sequence located in CNX2. a 80-89 and may interact with
SLTERCNLRC

SEQ ID NO: 334 - protein, 10 aa, Arabidopsis Thaliana
  Sequence located in RHA1. a 168-177 and may interact with
AKRLPRVQPA

SEQ ID NO: 335 - protein, 10 aa, Arabidopsis Thaliana
  Sequence located in CNX3. a 111-120 and may interact with
SDMSKLTHVG

SEQ ID NO: 336 - protein, 10 aa, Arabidopsis Thaliana
  Sequence located in CNX3. a 111-120 and may interact with
SDMSKLTHVG

SEQ ID NO: 337 - protein, 10 aa, Arabidopsis Thaliana
  Sequence located in CNX3. a 260-269 and may interact with
TGGKSGSWSR

SEQ ID NO: 338 - protein, 10 aa, Arabidopsis Thaliana
  Sequence located in CYP84A1 OR FAH1 OR F23E13.110. at 80-
SASFRSAPRP

SEQ ID NO: 339 - protein, 10 aa, Arabidopsis Thaliana
  Sequence located in CNX3. a 259-268 and may interact with
KTGGKSGSWS

SEQ ID NO: 340 - protein, 10 aa, Arabidopsis Thaliana
  Sequence located in CYP84A1 OR FAH1 OR F23E13.110. at 79-
LSASFRSAPR

SEQ ID NO: 341 - protein, 10 aa, Arabidopsis Thaliana
  Sequence located in CNX3. a 258-267 and may interact with
RKTGGKSGSW

SEQ ID NO: 342 - protein, 10 aa, Arabidopsis Thaliana
  Sequence located in CYP84A1 OR FAH1 OR F23E13.110. at 78-
SLSASFRSAP

SEQ ID NO: 343 - protein, 10 aa, Arabidopsis Thaliana
  Sequence located in CNX3. a 213-222 and may interact with
GEASCTGKTG

SEQ ID NO: 344 - protein, 10 aa, Arabidopsis Thaliana
  Sequence located in RER1C O T20D16.6. at 110-119 and may
ASLTSGGSLP

SEQ ID NO: 345 - protein, 10 aa, Arabidopsis Thaliana
  Sequence located in CNX3. a 134-143 and may interact with
DNSKRTALAC

SEQ ID NO: 346 - protein, 10 aa, Arabidopsis Thaliana
  Sequence located in ASA2 OR T27A16.21. at 322-331 and may
RGKGGPLTVV

SEQ ID NO: 347 - protein, 10 aa, Arabidopsis Thaliana
  Sequence located in CNX3. a 135-144 and may interact with
NSKRTALACC

SEQ ID NO: 348 - protein, 10 aa, Arabidopsis Thaliana
  Sequence located in ASA2 OR T27A16.21. at 321-330 and may
RRGKGGPLTV

SEQ ID NO: 349 - protein, 10 aa, Arabidopsis Thaliana
  Sequence located in COPT1. t 156-165 and may interact with
AGKEKRAVYA

SEQ ID NO: 350 - protein, 10 aa, Arabidopsis Thaliana
  Sequence located in ATJ. at 649-658 and may interact with
GVYGAFLLAC

SEQ ID NO: 351 - protein, 10 aa, Arabidopsis Thaliana
  Sequence located in COPT1. t 20-29 and may interact with
SGSLGALATT

SEQ ID NO: 352 - protein, 10 aa, Arabidopsis Thaliana
  Sequence located in Unknown at 5-14 and may interact with
RPTQSSEGSR

SEQ ID NO: 353 - protein, 10 aa, Arabidopsis Thaliana
  Sequence located in COPT1. t 45-54 and may interact with
LLSLGLIFIL

SEQ ID NO: 354 - protein, 10 aa, Arabidopsis Thaliana
  Sequence located in RPS6. a 212-221 and may interact with
EETKPEDEDE

SEQ ID NO: 355 - protein, 10 aa, Arabidopsis Thaliana
  Sequence located in CYP74A. at 40-49 and may interact with
DLTVATRTGS

SEQ ID NO: 356 - protein, 10 aa, Arabidopsis Thaliana
  Sequence located in COB OR YTB. at 125-134 and may interact
GAGTGSNGQV

SEQ ID NO: 357 - protein, 10 aa, Arabidopsis Thaliana
  Sequence located in CYP74A. at 327-336 and may interact with
ILFPNMVKRI

SEQ ID NO: 358 - protein, 10 aa, Arabidopsis Thaliana
  Sequence located in P2. at 61-270 and may interact with
NQEGVHNLSN

SEQ ID NO: 359 - protein, 10 aa, Arabidopsis Thaliana
  Sequence located in CYP84A1 OR FAH1 OR F23E13.110. at 45-
RTRRRSTAGP

SEQ ID NO: 360 - protein, 10 aa, Arabidopsis Thaliana
  Sequence located in CYP84A1 OR FAH1 OR F23E13.110. at 45-
RTRRRSTAGP

SEQ ID NO: 361 - protein, 10 aa, Arabidopsis Thaliana
  Sequence located in CYP84A1 OR FAH1 OR F23E13.110. at 45-
RTRRRSTAGP

SEQ ID NO: 362 - protein, 10 aa, Arabidopsis Thaliana
  Sequence located in RAB18. t 108-117 and may interact with
RSGSGSSSSS

SEQ ID NO: 363 - protein, 10 aa, Arabidopsis Thaliana
  Sequence located in CYP84A1 OR FAH1 OR F23E13.110. at 82-
SFRSAPRPAK

SEQ ID NO: 364 - protein, 10 aa, Arabidopsis Thaliana
  Sequence located in ILL2. a 205-214 and may interact with
GKAASRAGSF

SEQ ID NO: 365 - protein, 10 aa, Arabidopsis Thaliana
  Sequence located in CYP84A1 OR FAH1 OR F23E13.110. at 37-
PTSQALKTRT

SEQ ID NO: 366 - protein, 10 aa, Arabidopsis Thaliana
  Sequence located in ILR1. a 29-38 and may interact with
GSGLESLARG

SEQ ID NO: 367 - protein, 10 aa, Arabidopsis Thaliana
  Sequence located in CYP84A1 OR FAH1 OR F23E13.110. at 38-
TSQALKTRTR

SEQ ID NO: 368 - protein, 10 aa, Arabidopsis Thaliana
  Sequence located in ILR1. a 28-37 and may interact with
SGSGLESLAR

SEQ ID NO: 369 - protein, 10 aa, Arabidopsis Thaliana
  Sequence located in CYP84A1 OR FAH1 OR F23E13.110. at 44-
TRTRRRSTAG

SEQ ID NO: 370 - protein, 10 aa, Arabidopsis Thaliana
  Sequence located in Unknown at 48-57 and may interact with
SPSTASTSRT

SEQ ID NO: 371 - protein, 10 aa, Arabidopsis Thaliana
  Sequence located in CYP84A1 OR FAH1 OR F23E13.110. at 43-
KTRTRRRSTA

SEQ ID NO: 372 - protein, 10 aa, Arabidopsis Thaliana
  Sequence located in Unknown at 47-56 and may interact with
LSPSTASTSR

SEQ ID NO: 373 - protein, 10 aa, Arabidopsis Thaliana
  Sequence located in CYP84A1 OR FAH1 OR F23E13.110. at
PIEGFYLAGD

SEQ ID NO: 374 - protein, 10 aa, Arabidopsis Thaliana
  Sequence located in Unknown at 366-375 and may interact with
GDLAKVQRAV

SEQ ID NO: 375 - protein, 10 aa, Arabidopsis Thaliana
  Sequence located in CYP84A1 OR FAH1 OR F23E13.110. at
SPIEGFYLAG

SEQ ID NO: 376 - protein, 10 aa, Arabidopsis Thaliana
  Sequence located in Unknown at 365-374 and may interact with
GGDLAKVQRA

SEQ ID NO: 377 - protein, 10 aa, Arabidopsis Thaliana
  Sequence located in CYP84A1 OR FAH1 OR F23E13.110. at
RSPIEGFYLA

SEQ ID NO: 378 - protein, 10 aa, Arabidopsis Thaliana
  Sequence located in Unknown at 364-373 and may interact with
PGGDLAKVQR

SEQ ID NO: 379 - protein, 10 aa, Arabidopsis Thaliana
  Sequence located in CYP84A1 OR FAH1 OR F23E13.110. at
PIEGFYLAGD

SEQ ID NO: 380 - protein, 10 aa, Arabidopsis Thaliana
  Sequence located in (TUBA3 R TUA3) AND (TUBA5 OR
GDLAKVQRAV

SEQ ID NO: 381 - protein, 10 aa, Arabidopsis Thaliana
  Sequence located in CYP84A1 OR FAH1 OR F23E13.110. at
SPIEGFYLAG

SEQ ID NO: 382 - protein, 10 aa, Arabidopsis Thaliana
  Sequence located in (TUBA3 R TUA3) AND (TUBA5 OR
GGDLAKVQRA

SEQ ID NO: 383 - protein, 10 aa, Arabidopsis Thaliana
  Sequence located in CYP84A1 OR FAH1 OR F23E13.110. at
RSPIEGFYLA

SEQ ID NO: 384 - protein, 10 aa, Arabidopsis Thaliana
  Sequence located in (TUBA3 R TUA3) AND (TUBA5 OR
PGGDLAKVQR

SEQ ID NO: 385 - protein, 10 aa, Arabidopsis Thaliana
  Sequence located in CYP84A1 OR FAH1 OR F23E13.110. at
PIEGFYLAGD

SEQ ID NO: 386 - protein, 10 aa, Arabidopsis Thaliana
  Sequence located in TUBA6 O TUA6. at 366-375 and may
GDLAKVQRAV

SEQ ID NO: 387 - protein, 10 aa, Arabidopsis Thaliana
  Sequence located in CYP84A1 OR FAH1 OR F23E13.110. at
SPIEGFYLAG

SEQ ID NO: 388 - protein, 10 aa, Arabidopsis Thaliana
  Sequence located in TUBA6 O TUA6. at 365-374 and may
GGDLAKVQRA

SEQ ID NO: 389 - protein, 10 aa, Arabidopsis Thaliana
  Sequence located in CYP84A1 OR FAH1 OR F23E13.110. at
RSPIEGFYLA

SEQ ID NO: 390 - protein, 10 aa, Arabidopsis Thaliana
  Sequence located in TUBA6 O TUA6. at 364-373 and may
PGGDLAKVQR

SEQ ID NO: 391 - protein, 10 aa, Arabidopsis Thaliana
  Sequence located in CRY2 OR PHH1. at 562-571 and may
RELFSTAESS

SEQ ID NO: 392 - protein, 10 aa, Arabidopsis Thaliana
  Sequence located in CCT1. a 431-440 and may interact with
TTLGSREQLA

SEQ ID NO: 393 - protein, 10 aa, Arabidopsis Thaliana
  Sequence located in CRY2 OR PHH1. at 563-572 and may
ELFSTAESSS

SEQ ID NO: 394 - protein, 10 aa, Arabidopsis Thaliana
  Sequence located in CCT1. a 430-439 and may interact with
ATTLGSREQL

SEQ ID NO: 395 - protein, 10 aa, Arabidopsis Thaliana
  Sequence located in CTR1. a 261-270 and may interact with
RAVDSGVDSS

SEQ ID NO: 396 - protein, 10 aa, Arabidopsis Thaliana
  Sequence located in CTR1. a 261-270 and may interact with
RAVDSGVDSS

SEQ ID NO: 397 - protein, 10 aa, Arabidopsis Thaliana
  Sequence located in CTR1. a 149-158 and may interact with
ADSAGGSSSG

SEQ ID NO: 398 - protein, 10 aa, Arabidopsis Thaliana
  Sequence located in HAT7 OR ATHB-3. at 194-203 and may
SIRSATATTA

SEQ ID NO: 399 - protein, 10 aa, Arabidopsis Thaliana
  Sequence located in CTR1. a 148-157 and may interact with
AADSAGGSSS

SEQ ID NO: 400 - protein, 10 aa, Arabidopsis Thaliana
  Sequence located in HAT7 OR ATHB-3. at 193-202 and may
SSIRSATATT

SEQ ID NO: 401 - protein, 10 aa, Arabidopsis Thaliana
  Sequence located in CTR1. a 48-57 and may interact with
NTGKAKAERG

SEQ ID NO: 402 - protein, 10 aa, Arabidopsis Thaliana
  Sequence located in GL2. at 18-27 and may interact with
PALSLSLAGI

SEQ ID NO: 403 - protein, 10 aa, Arabidopsis Thaliana
  Sequence located in CTR1. a 49-58 and may interact with
TGKAKAERGG

SEQ ID NO: 404 - protein, 10 aa, Arabidopsis Thaliana
  Sequence located in GL2. at 17-26 and may interact with
SPALSLSLAG

SEQ ID NO: 405 - protein, 10 aa, Arabidopsis Thaliana
  Sequence located in CTR1. a 805-814 and may interact with
LRPLIKSAVP

SEQ ID NO: 406 - protein, 10 aa, Arabidopsis Thaliana
  Sequence located in T01O24. 7. at 57-66 and may interact with
RNCRFYKGAK

SEQ ID NO: 407 - protein, 10 aa, Arabidopsis Thaliana
  Sequence located in CTR1. a 633-642 and may interact with
LYRLLHKSGA

SEQ ID NO: 408 - protein, 10 aa, Arabidopsis Thaliana
  Sequence located in PAL1. a 551-560 and may interact with
QVAKKVLTTG

SEQ ID NO: 409 - protein, 10 aa, Arabidopsis Thaliana
  Sequence located in CTR1. a 132-141 and may interact with
FRLGFGGGGG

SEQ ID NO: 410 - protein, 10 aa, Arabidopsis Thaliana
  Sequence located in T29E15. 3. at 570-579 and may interact with
KPKPKAPAAA

SEQ ID NO: 411 - protein, 10 aa, Arabidopsis Thaliana
  Sequence located in CTR1. a 39-48 and may interact with
ENRSNHNSGN

SEQ ID NO: 412 - protein, 10 aa, Arabidopsis Thaliana
  Sequence located in T8F5.20 at 65-74 and may interact with
LVAGIVVAAI

SEQ ID NO: 413 - protein, 10 aa, Arabidopsis Thaliana
  Sequence located in COB OR YTB. at 123-132 and may interact
TIGAGTGSNG

SEQ ID NO: 414 - protein, 10 aa, Arabidopsis Thaliana
  Sequence located in COB OR YTB. at 123-132 and may interact
TIGAGTGSNG

SEQ ID NO: 415 - protein, 10 aa, Arabidopsis Thaliana
  Sequence located in COB OR YTB. at 61-70 and may interact
GRFLLPPKCK

SEQ ID NO: 416 - protein, 10 aa, Arabidopsis Thaliana
  Sequence located in PP1BG. t 295-304 and may interact with
ASEKKGRFGF

SEQ ID NO: 417 - protein, 10 aa, Arabidopsis Thaliana
  Sequence located in Unknown at 153-162 and may interact with
ITSLASAIPV

SEQ ID NO: 418 - protein, 10 aa, Arabidopsis Thaliana
  Sequence located in GAD1 OR GDH1 OR GAD. at 85-94 and
DRRESGRDRD

SEQ ID NO: 419 - protein, 10 aa, Arabidopsis Thaliana
  Sequence located in CC-1. a 47-56 and may interact with
RKTGSVAGYS

SEQ ID NO: 420 - protein, 10 aa, Arabidopsis Thaliana
  Sequence located in RPOMT. t 894-903 and may interact with
AVACNRAGLS

SEQ ID NO: 421 - protein, 10 aa, Arabidopsis Thaliana
  Sequence located in CYSJ. a 504-513 and may interact with
DCASLRRYPA

SEQ ID NO: 422 - protein, 10 aa, Arabidopsis Thaliana
  Sequence located in Unknown at 67-76 and may interact with
SGVSAEGSGV

SEQ ID NO: 423 - protein, 10 aa, Arabidopsis Thaliana
  Sequence located in CYSJ. a 194-203 and may interact with
SSDALNRLSA

SEQ ID NO: 424 - protein, 10 aa, Arabidopsis Thaliana
  Sequence located in HMG2 OR F5J6.13. at 178-187 and may
RREAVQRITG

SEQ ID NO: 425 - protein, 10 aa, Arabidopsis Thaliana
  Sequence located in Unknown at 339-348 and may interact with
SSGAAAAAAI

SEQ ID NO: 426 - protein, 10 aa, Arabidopsis Thaliana
  Sequence located in HAT9. a 232-241 and may interact with
NGGGGGGSGA

SEQ ID NO: 427 - protein, 10 aa, Arabidopsis Thaliana
  Sequence located in Unknown at 368-377 and may interact with
FGERYLSTQL

SEQ ID NO: 428 - protein, 10 aa, Arabidopsis Thaliana
  Sequence located in VALRS. t 671-680 and may interact with
KLGGEVPFSK

SEQ ID NO: 429 - protein, 10 aa, Arabidopsis Thaliana
  Sequence located in DRT100. at 18-27 and may interact with
PSDPLKRLSY

SEQ ID NO: 430 - protein, 10 aa, Arabidopsis Thaliana
  Sequence located in Unknown at 212-221 and may interact with
RAVGELAKAI

SEQ ID NO: 431 - protein, 10 aa, Arabidopsis Thaliana
  Sequence located in DRT100. at 17-26 and may interact with
SPSDPLKRLS

SEQ ID NO: 432 - protein, 10 aa, Arabidopsis Thaliana
  Sequence located in HMG1. a 204-213 and may interact with
REALQRVTGR

SEQ ID NO: 433 - protein, 10 aa, Arabidopsis Thaliana
  Sequence located in DRT102. at 88-97 and may interact with
KSTAGDSTTK

SEQ ID NO: 434 - protein, 10 aa, Arabidopsis Thaliana
  Sequence located in HMG1. a 443-452 and may interact with
LAGSAVAGSL

SEQ ID NO: 435 - protein, 10 aa, Arabidopsis Thaliana
  Sequence located in DRT102. at 57-66 and may interact with
KSPCPASGSE

SEQ ID NO: 436 - protein, 10 aa, Arabidopsis Thaliana
  Sequence located in Unknown at 388-397 and may interact with
LTRGGSTARL

SEQ ID NO: 437 - protein, 10 aa, Arabidopsis Thaliana
  Sequence located in DRT111. at 88-97 and may interact with
ALVGVTSSVI

SEQ ID NO: 438 - protein, 10 aa, Arabidopsis Thaliana
  Sequence located in GBF1. a 136-145 and may interact with
NDGASHSDES

SEQ ID NO: 439 - protein, 10 aa, Arabidopsis Thaliana
  Sequence located in DRT111. at 163-172 and may interact with
GEERGRDPAR

SEQ ID NO: 440 - protein, 10 aa, Arabidopsis Thaliana
  Sequence located in GBF1. a 46-55 and may interact with
PFFPSPVGSP

SEQ ID NO: 441 - protein, 10 aa, Arabidopsis Thaliana
  Sequence located in DRT111. at 242-251 and may interact with
AKKTDRRAGV

SEQ ID NO: 442 - protein, 10 aa, Arabidopsis Thaliana
  Sequence located in T29E15. 3. at 147-156 and may interact with
GLLSVTSGAN

SEQ ID NO: 443 - protein, 10 aa, Arabidopsis Thaliana
  Sequence located in DRT111. at 241-250 and may interact with
MAKKTDRRAG

SEQ ID NO: 444 - protein, 10 aa, Arabidopsis Thaliana
  Sequence located in T29E15. 3. at 146-155 and may interact with
HGLLSVTSGA

SEQ ID NO: 445 - protein, 10 aa, Arabidopsis Thaliana
  Sequence located in DRT111. at 240-249 and may interact with
LMAKKTDRRA

SEQ ID NO: 446 - protein, 10 aa, Arabidopsis Thaliana
  Sequence located in T29E15. 3. at 145-154 and may interact with
KHGLLSVTSG

SEQ ID NO: 447 - protein, 10 aa, Arabidopsis Thaliana
  Sequence located in DRT112. at 30-39 and may interact with
TQTAAVASPK

SEQ ID NO: 448 - protein, 10 aa, Arabidopsis Thaliana
  Sequence located in GRP7. a 211-220 and may interact with
GLGGGHGGGF

SEQ ID NO: 449 - protein, 10 aa, Arabidopsis Thaliana
  Sequence located in RD29A. t 62-71 and may interact with
GGVTGKPKSL

SEQ ID NO: 450 - protein, 10 aa, Arabidopsis Thaliana
  Sequence located in F4P9.44 OR T01B08.26. at 202-211 and may
ERFRFARDTS

SEQ ID NO: 451 - protein, 10 aa, Arabidopsis Thaliana
  Sequence located in DAD1. a 61-70 and may interact with
SGVLSCIGTA

SEQ ID NO: 452 - protein, 10 aa, Arabidopsis Thaliana
  Sequence located in Unknown at 381-390 and may interact with
RRSNAGENSG

SEQ ID NO: 453 - protein, 10 aa, Arabidopsis Thaliana
  Sequence located in DAD1. a 55-64 and may interact with
PFNSFLSGVL

SEQ ID NO: 454 - protein, 10 aa, Arabidopsis Thaliana
  Sequence located in RPL24. t 63-72 and may interact with
KDAAQEAVKR

SEQ ID NO: 455 - protein, 10 aa, Arabidopsis Thaliana
  Sequence located in DAD2. a 61-70 and may interact with
SGVLSCIGTA

SEQ ID NO: 456 - protein, 10 aa, Arabidopsis Thaliana
  Sequence located in Unknown at 381-390 and may interact with
RRSNAGENSG

SEQ ID NO: 457 - protein, 10 aa, Arabidopsis Thaliana
  Sequence located in DAD2. a 55-64 and may interact with
PFNSFLSGVL

SEQ ID NO: 458 - protein, 10 aa, Arabidopsis Thaliana
  Sequence located in RPL24. t 63-72 and may interact with
KDAAQEAVKR

SEQ ID NO: 459 - protein, 10 aa, Arabidopsis Thaliana
  Sequence located in GAD1 OR GDH1 OR GAD. at 571-580 and
REILIDENLS

SEQ ID NO: 460 - protein, 10 aa, Arabidopsis Thaliana
  Sequence located in GAD1 OR GDH1 OR GAD. at 571-580 and
REILIDENLS

SEQ ID NO: 461 - protein, 10 aa, Arabidopsis Thaliana
  Sequence located in GAD1 OR GDH1 OR GAD. at 572-581 and
EILIDENLSQ

SEQ ID NO: 462 - protein, 10 aa, Arabidopsis Thaliana
  Sequence located in GAD1 OR GDH1 OR GAD. at 570-579 and
LREILIDENL

SEQ ID NO: 463 - protein, 10 aa, Arabidopsis Thaliana
  Sequence located in GAD1 OR GDH1 OR GAD. at 789-798 and
QRAGRAGRTG

SEQ ID NO: 464 - protein, 10 aa, Arabidopsis Thaliana
  Sequence located in T06B20. . at 42-51 and may interact with
PSPSSSSSSL

SEQ ID NO: 465 - protein, 10 aa, Arabidopsis Thaliana
  Sequence located in GAD1 OR GDH1 OR GAD. at 350-359 and
PAFLQGQTRY

SEQ ID NO: 466 - protein, 10 aa, Arabidopsis Thaliana
  Sequence located in RPL12-B at 74-83 and may interact with
ISSLTLEESR

SEQ ID NO: 467 - protein, 10 aa, Arabidopsis Thaliana
  Sequence located in GAD1 OR GDH1 OR GAD. at 109-118 and
RDRERNRGDE

SEQ ID NO: 468 - protein, 10 aa, Arabidopsis Thaliana
  Sequence located in RPL28 O F4P9.22. at 27-36 and may
LISPISLSVT

SEQ ID NO: 469 - protein, 10 aa, Arabidopsis Thaliana
  Sequence located in GAD1 OR GDH1 OR GAD. at 69-78 and
REAEERRREE

SEQ ID NO: 470 - protein, 10 aa, Arabidopsis Thaliana
  Sequence located in Unknown at 10-19 and may interact with
SLSFFSSSLF

SEQ ID NO: 471 - protein, 10 aa, Arabidopsis Thaliana
  Sequence located in Unknown at 80-89 and may interact with
TYSTTDDSTT

SEQ ID NO: 472 - protein, 10 aa, Arabidopsis Thaliana
  Sequence located in PROC1. t 24-33 and may interact with
SIARGVVASG

SEQ ID NO: 473 - protein, 10 aa, Arabidopsis Thaliana
  Sequence located in Unknown at 79-88 and may interact with
LTYSTTDDST

SEQ ID NO: 474 - protein, 10 aa, Arabidopsis Thaliana
  Sequence located in PROC1. t 23-32 and may interact with
ESIARGVVAS

SEQ ID NO: 475 - protein, 10 aa, Arabidopsis Thaliana
  Sequence located in RAB18. t 33-42 and may interact with
GGGYGTGGGG

SEQ ID NO: 476 - protein, 10 aa, Arabidopsis Thaliana
  Sequence located in FBP. at 44-53 and may interact with
SASASTVSAP

SEQ ID NO: 477 - protein, 10 aa, Arabidopsis Thaliana
  Sequence located in RAB18. t 68-77 and may interact with
GQGYGTGTGT

SEQ ID NO: 478 - protein, 10 aa, Arabidopsis Thaliana
  Sequence located in Unknown at 28-37 and may interact with
RSRSCPITLT

SEQ ID NO: 479 - protein, 10 aa, Arabidopsis Thaliana
  Sequence located in RAB18. t 33-42 and may interact with
GGGYGTGGGG

SEQ ID NO: 480 - protein, 10 aa, Arabidopsis Thaliana
  Sequence located in ERD11 O GST1. at 8-17 and may interact
TSTISSSSPS

SEQ ID NO: 481 - protein, 10 aa, Arabidopsis Thaliana
  Sequence located in RAB18. t 37-46 and may interact with
GTGGGGGATG

SEQ ID NO: 482 - protein, 10 aa, Arabidopsis Thaliana
  Sequence located in T06B20. . at 35-44 and may interact with
SSSPPPSPSP

SEQ ID NO: 483 - protein, 10 aa, Arabidopsis Thaliana
  Sequence located in RAB18. t 38-47 and may interact with
TGGGGGATGG

SEQ ID NO: 484 - protein, 10 aa, Arabidopsis Thaliana
  Sequence located in T06B20. . at 34-43 and may interact with
ASSSPPPSPS

SEQ ID NO: 485 - protein, 10 aa, Arabidopsis Thaliana
  Sequence located in RAB18. t 40-49 and may interact with
GGGGATGGQG

SEQ ID NO: 486 - protein, 10 aa, Arabidopsis Thaliana
  Sequence located in T06B20. . at 43-52 and may interact with
SPSSSSSSLS

SEQ ID NO: 487 - protein, 10 aa, Arabidopsis Thaliana
  Sequence located in RAB18. t 39-48 and may interact with
GGGGGATGGQ

SEQ ID NO: 488 - protein, 10 aa, Arabidopsis Thaliana
  Sequence located in T06B20. . at 42-51 and may interact with
PSPSSSSSSL

SEQ ID NO: 489 - protein, 10 aa, Arabidopsis Thaliana
  Sequence located in RAB18. t 38-47 and may interact with
TGGGGGATGG

SEQ ID NO: 490 - protein, 10 aa, Arabidopsis Thaliana
  Sequence located in T06B20. . at 41-50 and may interact with
SPSPSSSSSS

SEQ ID NO: 491 - protein, 10 aa, Arabidopsis Thaliana
  Sequence located in RAB18. t 37-46 and may interact with
GTGGGGGATG

SEQ ID NO: 492 - protein, 10 aa, Arabidopsis Thaliana
  Sequence located in T06B20. . at 40-49 and may interact with
PSPSPSSSSS

SEQ ID NO: 493 - protein, 10 aa, Arabidopsis Thaliana
  Sequence located in RAB18. t 107-116 and may interact with
HRSGSGSSSS

SEQ ID NO: 494 - protein, 10 aa, Arabidopsis Thaliana
  Sequence located in HAT9. a 235-244 and may interact with
GGGGSGATAV

SEQ ID NO: 495 - protein, 10 aa, Arabidopsis Thaliana
  Sequence located in RAB18. t 108-117 and may interact with
RSGSGSSSSS

SEQ ID NO: 496 - protein, 10 aa, Arabidopsis Thaliana
  Sequence located in HAT9. a 234-243 and may interact with
GGGGGSGATA

SEQ ID NO: 497 - protein, 10 aa, Arabidopsis Thaliana
  Sequence located in RAB18. t 108-117 and may interact with
RSGSGSSSSS

SEQ ID NO: 498 - protein, 10 aa, Arabidopsis Thaliana
  Sequence located in HMG1. a 583-592 and may interact with
GATTTTTTTT

SEQ ID NO: 499 - protein, 10 aa, Arabidopsis Thaliana
  Sequence located in RAB18. t 108-117 and may interact with
RSGSGSSSSS

SEQ ID NO: 500 - protein, 10 aa, Arabidopsis Thaliana
  Sequence located in HMG1. a 582-591 and may interact with
SGATTTTTTT

SEQ ID NO: 501 - protein, 10 aa, Arabidopsis Thaliana
  Sequence located in RAB18. t 38-47 and may interact with
TGGGGGATGG

SEQ ID NO: 502 - protein, 10 aa, Arabidopsis Thaliana
  Sequence located in P5CSB O P5CS2. at 20-29 and may interact
SAPATSGGAT

SEQ ID NO: 503 - protein, 10 aa, Arabidopsis Thaliana
  Sequence located in RAB18. t 37-46 and may interact with
GTGGGGGATG

SEQ ID NO: 504 - protein, 10 aa, Arabidopsis Thaliana
  Sequence located in P5CSB O P5CS2. at 19-28 and may interact
TSAPATSGGA

SEQ ID NO: 505 - protein, 10 aa, Arabidopsis Thaliana
  Sequence located in RAB18. t 36-45 and may interact with
YGTGGGGGAT

SEQ ID NO: 506 - protein, 10 aa, Arabidopsis Thaliana
  Sequence located in P5CSB O P5CS2. at 18-27 and may interact
VTSAPATSGG

SEQ ID NO: 507 - protein, 10 aa, Arabidopsis Thaliana
  Sequence located in RAB18. t 35-44 and may interact with
GYGTGGGGGA

SEQ ID NO: 508 - protein, 10 aa, Arabidopsis Thaliana
  Sequence located in P5CSB O P5CS2. at 17-26 and may interact
AVTSAPATSG

SEQ ID NO: 509 - protein, 10 aa, Arabidopsis Thaliana
  Sequence located in RAB18. t 34-43 and may interact with
GGYGTGGGGG

SEQ ID NO: 510 - protein, 10 aa, Arabidopsis Thaliana
  Sequence located in P5CSB O P5CS2. at 16-25 and may interact
AAVTSAPATS

SEQ ID NO: 511 - protein, 10 aa, Arabidopsis Thaliana
  Sequence located in RAB18. t 33-42 and may interact with
GGGYGTGGGG

SEQ ID NO: 512 - protein, 10 aa, Arabidopsis Thaliana
  Sequence located in P5CSB O P5CS2. at 15-24 and may interact
TAAVTSAPAT

SEQ ID NO: 513 - protein, 10 aa, Arabidopsis Thaliana
  Sequence located in RAB18. t 32-41 and may interact with
GGGGYGTGGG

SEQ ID NO: 514 - protein, 10 aa, Arabidopsis Thaliana
  Sequence located in P5CSB O P5CS2. at 14-23 and may interact
STAAVTSAPA

SEQ ID NO: 515 - protein, 10 aa, Arabidopsis Thaliana
  Sequence located in RAB18. t 61-70 and may interact with
GQGYGTGGQG

SEQ ID NO: 516 - protein, 10 aa, Arabidopsis Thaliana
  Sequence located in PSBY. a 42-51 and may interact with
SLAVTSTALA

SEQ ID NO: 517 - protein, 10 aa, Arabidopsis Thaliana
  Sequence located in RAB18. t 47-56 and may interact with
GQGYGTGGQG

SEQ ID NO: 518 - protein, 10 aa, Arabidopsis Thaliana
  Sequence located in PSBY. a 42-51 and may interact with
SLAVTSTALA

SEQ ID NO: 519 - protein, 10 aa, Arabidopsis Thaliana
  Sequence located in RAB18. t 51-60 and may interact with
GTGGQGYGSG

SEQ ID NO: 520 - protein, 10 aa, Arabidopsis Thaliana
  Sequence located in Unknown at 2-11 and may interact with
ASTTLSIATT

SEQ ID NO: 521 - protein, 10 aa, Arabidopsis Thaliana
  Sequence located in RAB18. t 51-60 and may interact with
GTGGQGYGSG

SEQ ID NO: 522 - protein, 10 aa, Arabidopsis Thaliana
  Sequence located in RPL12-C at 2-11 and may interact with
ASTTLSIATT

SEQ ID NO: 523 - protein, 10 aa, Arabidopsis Thaliana
  Sequence located in RAB18. t 108-117 and may interact with
RSGSGSSSSS

SEQ ID NO: 524 - protein, 10 aa, Arabidopsis Thaliana
  Sequence located in RER1C O T20D16.6. at 12-21 and may
AAAATTATAT

SEQ ID NO: 525 - protein, 10 aa, Arabidopsis Thaliana
  Sequence located in RAB18. t 108-117 and may interact with
RSGSGSSSSS

SEQ ID NO: 526 - protein, 10 aa, Arabidopsis Thaliana
  Sequence located in RER1C O T20D16.6. at 12-21 and may
AAAATTATAT

SEQ ID NO: 527 - protein, 10 aa, Arabidopsis Thaliana
  Sequence located in RAB18. t 108-117 and may interact with
RSGSGSSSSS

SEQ ID NO: 528 - protein, 10 aa, Arabidopsis Thaliana
  Sequence located in RER1C O T20D16.6. at 11-20 and may
PAAAATTATA

SEQ ID NO: 529 - protein, 10 aa, Arabidopsis Thaliana
  Sequence located in RAB18. t 37-46 and may interact with
GTGGGGGATG

SEQ ID NO: 530 - protein, 10 aa, Arabidopsis Thaliana
  Sequence located in RPOPT. t 56-65 and may interact with
SSSSSSSSST

SEQ ID NO: 531 - protein, 10 aa, Arabidopsis Thaliana
  Sequence located in RAB18. t 37-46 and may interact with
GTGGGGGATG

SEQ ID NO: 532 - protein, 10 aa, Arabidopsis Thaliana
  Sequence located in RPOPT. t 55-64 and may interact with
ASSSSSSSSS

SEQ ID NO: 533 - protein, 10 aa, Arabidopsis Thaliana
  Sequence located in RAB18. t 38-47 and may interact with
TGGGGGATGG

SEQ ID NO: 534 - protein, 10 aa, Arabidopsis Thaliana
  Sequence located in RPOPT. t 57-66 and may interact with
SSSSSSSSTS

SEQ ID NO: 535 - protein, 10 aa, Arabidopsis Thaliana
  Sequence located in RAB18. t 38-47 and may interact with
TGGGGGATGG

SEQ ID NO: 536 - protein, 10 aa, Arabidopsis Thaliana
  Sequence located in RPOPT. t 55-64 and may interact with
ASSSSSSSSS

SEQ ID NO: 537 - protein, 10 aa, Arabidopsis Thaliana
  Sequence located in RAB18. t 38-47 and may interact with
TGGGGGATGG

SEQ ID NO: 538 - protein, 10 aa, Arabidopsis Thaliana
  Sequence located in RPOPT. t 54-63 and may interact with
SASSSSSSSS

SEQ ID NO: 539 - protein, 10 aa, Arabidopsis Thaliana
  Sequence located in RAB18. t 41-50 and may interact with
GGGATGGQGY

SEQ ID NO: 540 - protein, 10 aa, Arabidopsis Thaliana
  Sequence located in RPOPT. t 60-69 and may interact with
SSSSSTSLSV

SEQ ID NO: 541 - protein, 10 aa, Arabidopsis Thaliana
  Sequence located in RAB18. t 40-49 and may interact with
GGGGATGGQG

SEQ ID NO: 542 - protein, 10 aa, Arabidopsis Thaliana
  Sequence located in RPOPT. t 59-68 and may interact with
SSSSSSTSLS

SEQ ID NO: 543 - protein, 10 aa, Arabidopsis Thaliana
  Sequence located in RAB18. t 39-48 and may interact with
GGGGGATGGQ

SEQ ID NO: 544 - protein, 10 aa, Arabidopsis Thaliana
  Sequence located in RPOPT. t 58-67 and may interact with
SSSSSSSTSL

SEQ ID NO: 545 - protein, 10 aa, Arabidopsis Thaliana
  Sequence located in RAB18. t 38-47 and may interact with
TGGGGGATGG

SEQ ID NO: 546 - protein, 10 aa, Arabidopsis Thaliana
  Sequence located in RPOPT. t 57-66 and may interact with
SSSSSSSSTS

SEQ ID NO: 547 - protein, 10 aa, Arabidopsis Thaliana
  Sequence located in RAB18. t 38-47 and may interact with
TGGGGGATGG

SEQ ID NO: 548 - protein, 10 aa, Arabidopsis Thaliana
  Sequence located in RPOPT. t 56-65 and may interact with
SSSSSSSSST

SEQ ID NO: 549 - protein, 10 aa, Arabidopsis Thaliana
  Sequence located in RAB18. t 38-47 and may interact with
TGGGGGATGG

SEQ ID NO: 550 - protein, 10 aa, Arabidopsis Thaliana
  Sequence located in RPOPT. t 54-63 and may interact with
SASSSSSSSS

SEQ ID NO: 551 - protein, 10 aa, Arabidopsis Thaliana
  Sequence located in RAB18. t 37-46 and may interact with
GTGGGGGATG

SEQ ID NO: 552 - protein, 10 aa, Arabidopsis Thaliana
  Sequence located in RPOPT. t 56-65 and may interact with
SSSSSSSSST

SEQ ID NO: 553 - protein, 10 aa, Arabidopsis Thaliana
  Sequence located in RAB18. t 37-46 and may interact with
GTGGGGGATG

SEQ ID NO: 554 - protein, 10 aa, Arabidopsis Thaliana
  Sequence located in RPOPT. t 55-64 and may interact with
ASSSSSSSSS

SEQ ID NO: 555 - protein, 10 aa, Arabidopsis Thaliana
  Sequence located in RAB18. t 35-44 and may interact with
GYGTGGGGGA

SEQ ID NO: 556 - protein, 10 aa, Arabidopsis Thaliana
  Sequence located in Unknown at 38-47 and may interact with
SSSSTSSSVA

SEQ ID NO: 557 - protein, 10 aa, Arabidopsis Thaliana
  Sequence located in RAB18. t 36-45 and may interact with
YGTGGGGGAT

SEQ ID NO: 558 - protein, 10 aa, Arabidopsis Thaliana
  Sequence located in Unknown at 37-46 and may interact with
SSSSSTSSSV

SEQ ID NO: 559 - protein, 10 aa, Arabidopsis Thaliana
  Sequence located in RAB18. t 35-44 and may interact with
GYGTGGGGGA

SEQ ID NO: 560 - protein, 10 aa, Arabidopsis Thaliana
  Sequence located in Unknown at 13-22 and may interact with
SVSSAPSSSS

SEQ ID NO: 561 - protein, 10 aa, Arabidopsis Thaliana
  Sequence located in RAB18. t 34-43 and may interact with
GGYGTGGGGG

SEQ ID NO: 562 - protein, 10 aa, Arabidopsis Thaliana
  Sequence located in Unknown at 12-21 and may interact with
ASVSSAPSSS

SEQ ID NO: 563 - protein, 10 aa, Arabidopsis Thaliana
  Sequence located in COR47 O RD17. at 105-114 and may
HRSNSSSSSS

SEQ ID NO: 564 - protein, 10 aa, Arabidopsis Thaliana
  Sequence located in T20D16. 2. at 43-52 and may interact with
TARGGRVTAM

SEQ ID NO: 565 - protein, 10 aa, Arabidopsis Thaliana
  Sequence located in COR47 O RD17. at 106-115 and may
RSNSSSSSSS

SEQ ID NO: 566 - protein, 10 aa, Arabidopsis Thaliana
  Sequence located in PAB5. a 27-36 and may interact with
AAVAAAAAAA

SEQ ID NO: 567 - protein, 10 aa, Arabidopsis Thaliana
  Sequence located in COR47 O RD17. at 105-114 and may
HRSNSSSSSS

SEQ ID NO: 568 - protein, 10 aa, Arabidopsis Thaliana
  Sequence located in PAB5. a 26-35 and may interact with
VAAVAAAAAA

SEQ ID NO: 569 - protein, 10 aa, Arabidopsis Thaliana
  Sequence located in COR47 O RD17. at 104-113 and may
LHRSNSSSSS

SEQ ID NO: 570 - protein, 10 aa, Arabidopsis Thaliana
  Sequence located in PAB5. a 25-34 and may interact with
QVAAVAAAAA

SEQ ID NO: 571 - protein, 10 aa, Arabidopsis Thaliana
  Sequence located in XERO1. t 78-87 and may interact with
ILCACCSTSC

SEQ ID NO: 572 - protein, 10 aa, Arabidopsis Thaliana
  Sequence located in Unknown at 79-88 and may interact with
DKRSGGGSRR

SEQ ID NO: 573 - protein, 10 aa, Arabidopsis Thaliana
  Sequence located in XERO1. t 77-86 and may interact with
CILCACCSTS

SEQ ID NO: 574 - protein, 10 aa, Arabidopsis Thaliana
  Sequence located in Unknown at 78-87 and may interact with
GDKRSGGGSR

SEQ ID NO: 575 - protein, 10 aa, Arabidopsis Thaliana
  Sequence located in Unknown at 121-130 and may interact with
SHQTGTNTAY

SEQ ID NO: 576 - protein, 10 aa, Arabidopsis Thaliana
  Sequence located in Unknown at 40-49 and may interact with
RVLCSSVSSV

SEQ ID NO: 577 - protein, 10 aa, Arabidopsis Thaliana
  Sequence located in Unknown at 83-92 and may interact with
SHQTGTNTTY

SEQ ID NO: 578 - protein, 10 aa, Arabidopsis Thaliana
  Sequence located in Unknown at 40-49 and may interact with
RVLCSSVSSV

SEQ ID NO: 579 - protein, 10 aa, Arabidopsis Thaliana
  Sequence located in DIM OR WF1. at 195-204 and may interact
ELVRATRDNE

SEQ ID NO: 580 - protein, 10 aa, Arabidopsis Thaliana
  Sequence located in P5CSA O P5CS1 OR T5I7.10. at 486-495
LVIPRGSNKL

SEQ ID NO: 581 - protein, 10 aa, Arabidopsis Thaliana
  Sequence located in DIM OR WF1. at 457-466 and may interact
DVGVYYAPGC

SEQ ID NO: 582 - protein, 10 aa, Arabidopsis Thaliana
  Sequence located in T09D09. 3. at 331-340 and may interact with
INAHVISGPG

SEQ ID NO: 583 - protein, 10 aa, Arabidopsis Thaliana
  Sequence located in ATJ. at 209-218 and may interact with
ATTPEDLVAT

SEQ ID NO: 584 - protein, 10 aa, Arabidopsis Thaliana
  Sequence located in Unknown at 251-260 and may interact with
RGCGFVKYSS

SEQ ID NO: 585 - protein, 10 aa, Arabidopsis Thaliana
  Sequence located in ATJ. at 653-662 and may interact with
AFLLACYDVD

SEQ ID NO: 586 - protein, 10 aa, Arabidopsis Thaliana
  Sequence located in MPK6 OR F18O19.10. at 247-256 and may
INVVGSQQES

SEQ ID NO: 587 - protein, 10 aa, Arabidopsis Thaliana
  Sequence located in Unknown at 54-63 and may interact with
SLVLEKELSS

SEQ ID NO: 588 - protein, 10 aa, Arabidopsis Thaliana
  Sequence located in Unknown at 117-126 and may interact with
TRELFLKNKA

SEQ ID NO: 589 - protein, 10 aa, Arabidopsis Thaliana
  Sequence located in Unknown at 162-171 and may interact with
LGVLACPNLP

SEQ ID NO: 590 - protein, 10 aa, Arabidopsis Thaliana
  Sequence located in CKA1. a 38-47 and may interact with
RKVGRGKYSE

SEQ ID NO: 591 - protein, 10 aa, Arabidopsis Thaliana
  Sequence located in Unknown at 263-272 and may interact with
YGALSRGDGA

SEQ ID NO: 592 - protein, 10 aa, Arabidopsis Thaliana
  Sequence located in SRP-54B at 261-270 and may interact with
SAVAATKSPV

SEQ ID NO: 593 - protein, 10 aa, Arabidopsis Thaliana
  Sequence located in Unknown at 263-272 and may interact with
YGALSRGDGA

SEQ ID NO: 594 - protein, 10 aa, Arabidopsis Thaliana
  Sequence located in SRP-54C at 261-270 and may interact with
SAVAATKSPV

SEQ ID NO: 595 - protein, 10 aa, Arabidopsis Thaliana
  Sequence located in Unknown at 88-97 and may interact with
AVATPPAADS

SEQ ID NO: 596 - protein, 10 aa, Arabidopsis Thaliana
  Sequence located in HAT9. a 224-233 and may interact with
RIGGGGGGNG

SEQ ID NO: 597 - protein, 10 aa, Arabidopsis Thaliana
  Sequence located in Unknown at 17-26 and may interact with
CSYSSPSPSL

SEQ ID NO: 598 - protein, 10 aa, Arabidopsis Thaliana
  Sequence located in APP. at 48-57 and may interact with
EGGGGTTITT

SEQ ID NO: 599 - protein, 10 aa, Arabidopsis Thaliana
  Sequence located in Unknown at 5-14 and may interact with
APAACSSSSR

SEQ ID NO: 600 - protein, 10 aa, Arabidopsis Thaliana
  Sequence located in RLK5 OR F21O9.180. at 43-52 and may
SGGGGGGGAP

SEQ ID NO: 601 - protein, 10 aa, Arabidopsis Thaliana
  Sequence located in Unknown at 4-13 and may interact with
SAPAACSSSS

SEQ ID NO: 602 - protein, 10 aa, Arabidopsis Thaliana
  Sequence located in RLK5 OR F21O9.180. at 42-51 and may
TSGGGGGGGA

SEQ ID NO: 603 - protein, 10 aa, Arabidopsis Thaliana
  Sequence located in Unknown at 4-13 and may interact with
SAPAACSSSS

SEQ ID NO: 604 - protein, 10 aa, Arabidopsis Thaliana
  Sequence located in Unknown at 78-87 and may interact with
ARRRRGCWRR

SEQ ID NO: 605 - protein, 10 aa, Arabidopsis Thaliana
  Sequence located in Unknown at 564-573 and may interact with
IESDGLGKGC

SEQ ID NO: 606 - protein, 10 aa, Arabidopsis Thaliana
  Sequence located in RPS11. t 130-139 and may interact with
RPLSKTVRFN

SEQ ID NO: 607 - protein, 10 aa, Arabidopsis Thaliana
  Sequence located in Unknown at 565-574 and may interact with
ESDGLGKGCT

SEQ ID NO: 608 - protein, 10 aa, Arabidopsis Thaliana
  Sequence located in RPS11. t 129-138 and may interact with
CRPLSKTVRF

SEQ ID NO: 609 - protein, 10 aa, Arabidopsis Thaliana
  Sequence located in Unknown at 487-496 and may interact with
TKSDTRAADF

SEQ ID NO: 610 - protein, 10 aa, Arabidopsis Thaliana
  Sequence located in PAT1. a 25-34 and may interact with
SLAVSSSSVE

SEQ ID NO: 611 - protein, 10 aa, Arabidopsis Thaliana
  Sequence located in Unknown at 379-388 and may interact with
RLMVETILKS

SEQ ID NO: 612 - protein, 10 aa, Arabidopsis Thaliana
  Sequence located in F4P9.44 OR T01B08.26. at 384-393 and may
TLKNCFHHKT

SEQ ID NO: 613 - protein, 10 aa, Arabidopsis Thaliana
  Sequence located in FBP. at 44-53 and may interact with
SASASTVSAP

SEQ ID NO: 614 - protein, 10 aa, Arabidopsis Thaliana
  Sequence located in GRP7. a 286-295 and may interact with
GGAGGGHGGG

SEQ ID NO: 615 - protein, 10 aa, Arabidopsis Thaliana
  Sequence located in FBP. at 44-53 and may interact with
SASASTVSAP

SEQ ID NO: 616 - protein, 10 aa, Arabidopsis Thaliana
  Sequence located in HAT9. a 229-238 and may interact with
GGGNGGGGGG

SEQ ID NO: 617 - protein, 10 aa, Arabidopsis Thaliana
  Sequence located in FBP. at 44-53 and may interact with
SASASTVSAP

SEQ ID NO: 618 - protein, 10 aa, Arabidopsis Thaliana
  Sequence located in HAT9. a 226-235 and may interact with
GGGGGGNGGG

SEQ ID NO: 619 - protein, 10 aa, Arabidopsis Thaliana
  Sequence located in FBP. at 223-232 and may interact with
ACATSNYCFY

SEQ ID NO: 620 - protein, 10 aa, Arabidopsis Thaliana
  Sequence located in SRP-54B at 406-415 and may interact with
RGSGRIVREV

SEQ ID NO: 621 - protein, 10 aa, Arabidopsis Thaliana
  Sequence located in Unknown at 383-392 and may interact with
HTIATSGFGA

SEQ ID NO: 622 - protein, 10 aa, Arabidopsis Thaliana
  Sequence located in PETE. a 239-248 and may interact with
VRDSRRPKPG

SEQ ID NO: 623 - protein, 10 aa, Arabidopsis Thaliana
  Sequence located in Unknown at 192-201 and may interact with
HIRGGGFKNV

SEQ ID NO: 624 - protein, 10 aa, Arabidopsis Thaliana
  Sequence located in RNS2 OR T5I7.8. at 195-204 and may
VDTSASKFIN

SEQ ID NO: 625 - protein, 10 aa, Arabidopsis Thaliana
  Sequence located in Unknown at 171-180 and may interact with
LAYDITAACS

SEQ ID NO: 626 - protein, 10 aa, Arabidopsis Thaliana
  Sequence located in F21J9.1 . at 412-421 and may interact with
KSIVYGGGAA

SEQ ID NO: 627 - protein, 10 aa, Arabidopsis Thaliana
  Sequence located in Unknown at 67-76 and may interact with
PSGCKLIGCG

SEQ ID NO: 628 - protein, 10 aa, Arabidopsis Thaliana
  Sequence located in Unknown at 240-249 and may interact with
GTATLKDATS

SEQ ID NO: 629 - protein, 10 aa, Arabidopsis Thaliana
  Sequence located in Unknown at 76-85 and may interact with
GSAVPSLLIS

SEQ ID NO: 630 - protein, 10 aa, Arabidopsis Thaliana
  Sequence located in UBC7 OR UBC14. at 137-146 and may
TARNWTQKYA

SEQ ID NO: 631 - protein, 10 aa, Arabidopsis Thaliana
  Sequence located in Unknown at 75-84 and may interact with
CGSAVPSLLI

SEQ ID NO: 632 - protein, 10 aa, Arabidopsis Thaliana
  Sequence located in UBC7 OR UBC14. at 136-145 and may
ATARNWTQKY

SEQ ID NO: 633 - protein, 10 aa, Arabidopsis Thaliana
  Sequence located in Unknown at 382-391 and may interact with
GHTIATSGFG

SEQ ID NO: 634 - protein, 10 aa, Arabidopsis Thaliana
  Sequence located in Unknown at 24-33 and may interact with
SKPAGSDSVS

SEQ ID NO: 635 - protein, 10 aa, Arabidopsis Thaliana
  Sequence located in Unknown at 285-294 and may interact with
IVRFAEPKRP

SEQ ID NO: 636 - protein, 10 aa, Arabidopsis Thaliana
  Sequence located in F4P9.44 OR T01B08.26. at 199-208 and may
NDPERFRFAR

SEQ ID NO: 637 - protein, 10 aa, Arabidopsis Thaliana
  Sequence located in FAD6 OR FADC. at 10-19 and may interact
PTSSKKSETD

SEQ ID NO: 638 - protein, 10 aa, Arabidopsis Thaliana
  Sequence located in Unknown at 837-846 and may interact with
VSFAFFTRGG

SEQ ID NO: 639 - protein, 10 aa, Arabidopsis Thaliana
  Sequence located in T4L20.2 0. at 311-320 and may interact with
GVVKLRRGLT

SEQ ID NO: 640 - protein, 10 aa, Arabidopsis Thaliana
  Sequence located in Unknown at 86-95 and may interact with
GETPSELNHS

SEQ ID NO: 641 - protein, 10 aa, Arabidopsis Thaliana
  Sequence located in T20D16. 2. at 89-98 and may interact with
YSCRAGSCSS

SEQ ID NO: 642 - protein, 10 aa, Arabidopsis Thaliana
  Sequence located in HAT9. a 235-244 and may interact with
GGGGSGATAV

SEQ ID NO: 643 - protein, 10 aa, Arabidopsis Thaliana
  Sequence located in T20D16. 2. at 90-99 and may interact with
SCRAGSCSSC

SEQ ID NO: 644 - protein, 10 aa, Arabidopsis Thaliana
  Sequence located in HAT9. a 234-243 and may interact with
GGGGGSGATA

SEQ ID NO: 645 - protein, 10 aa, Arabidopsis Thaliana
  Sequence located in T20D16. 2. at 91-100 and may interact with
CRAGSCSSCA

SEQ ID NO: 646 - protein, 10 aa, Arabidopsis Thaliana
  Sequence located in HAT9. a 233-242 and may interact with
GGGGGGSGAT

SEQ ID NO: 647 - protein, 10 aa, Arabidopsis Thaliana
  Sequence located in T20D16. 2. at 90-99 and may interact with
SCRAGSCSSC

SEQ ID NO: 648 - protein, 10 aa, Arabidopsis Thaliana
  Sequence located in ORC2 OR F13M22.6. at 37-46 and may
AAASPGGGRR

SEQ ID NO: 649 - protein, 10 aa, Arabidopsis Thaliana
  Sequence located in T20D16. 2. at 91-100 and may interact with
CRAGSCSSCA

SEQ ID NO: 650 - protein, 10 aa, Arabidopsis Thaliana
  Sequence located in VPS41. t 325-334 and may interact with
CTTAAAASAR

SEQ ID NO: 651 - protein, 10 aa, Arabidopsis Thaliana
  Sequence located in FLO. at 3-12 and may interact with Sequence
TDLKSTFLNV

SEQ ID NO: 652 - protein, 10 aa, Arabidopsis Thaliana
  Sequence located in (AVA-P1 OR T4L20.300) AND AVA-P2
DIEEGTLEIG

SEQ ID NO: 653 - protein, 10 aa, Arabidopsis Thaliana
  Sequence located in GAPA. a 18-27 and may interact with
LRSSSASLPF

SEQ ID NO: 654 - protein, 10 aa, Arabidopsis Thaliana
  Sequence located in GBF3 OR T3F17.8. at 199-208 and may
ESAARSRERK

SEQ ID NO: 655 - protein, 10 aa, Arabidopsis Thaliana
  Sequence located in GASA1. t 47-56 and may interact with
RCRLSRRPRL

SEQ ID NO: 656 - protein, 10 aa, Arabidopsis Thaliana
  Sequence located in PYRB. a 190-199 and may interact with
ESGAARKAAA

SEQ ID NO: 657 - protein, 10 aa, Arabidopsis Thaliana
  Sequence located in GASA2. t 85-94 and may interact with
CYASITTHGG

SEQ ID NO: 658 - protein, 10 aa, Arabidopsis Thaliana
  Sequence located in KATB. a 549-558 and may interact with
AVRTDSGVSP

SEQ ID NO: 659 - protein, 10 aa, Arabidopsis Thaliana
  Sequence located in GASA2. t 56-65 and may interact with
KLCLRACNSC

SEQ ID NO: 660 - protein, 10 aa, Arabidopsis Thaliana
  Sequence located in NDPK1. t 176-185 and may interact with
LQAEPGTIRG

SEQ ID NO: 661 - protein, 10 aa, Arabidopsis Thaliana
  Sequence located in GASA3. t 40-49 and may interact with
DCGGRCKGRC

SEQ ID NO: 662 - protein, 10 aa, Arabidopsis Thaliana
  Sequence located in ERD11 O GST1. at 106-115 and may
ATSFGASAAV

SEQ ID NO: 663 - protein, 10 aa, Arabidopsis Thaliana
  Sequence located in GASA3. t 60-69 and may interact with
RACNSCCYRC

SEQ ID NO: 664 - protein, 10 aa, Arabidopsis Thaliana
  Sequence located in Unknown at 84-93 and may interact with
SSAVRGGVTG

SEQ ID NO: 665 - protein, 10 aa, Arabidopsis Thaliana
  Sequence located in GASA3. t 59-68 and may interact with
LRACNSCCYR

SEQ ID NO: 666 - protein, 10 aa, Arabidopsis Thaliana
  Sequence located in Unknown at 83-92 and may interact with
ESSAVRGGVT

SEQ ID NO: 667 - protein, 10 aa, Arabidopsis Thaliana
  Sequence located in GPA1 OR T1D16.6. at 23-32 and may
QLRQRRLQLL

SEQ ID NO: 668 - protein, 10 aa, Arabidopsis Thaliana
  Sequence located in GBF3 OR T3F17.8. at 215-224 and may
LETLAAKLEE

SEQ ID NO: 669 - protein, 10 aa, Arabidopsis Thaliana
  Sequence located in GPA1 OR T1D16.6. at 132-141 and may
SIFSLSSTAD

SEQ ID NO: 670 - protein, 10 aa, Arabidopsis Thaliana
  Sequence located in RPL17. t 57-66 and may interact with
VGRTAQAKNR

SEQ ID NO: 671 - protein, 10 aa, Arabidopsis Thaliana
  Sequence located in GPA1 OR T1D16.6. at 297-306 and may
VTSIAFSVSG

SEQ ID NO: 672 - protein, 10 aa, Arabidopsis Thaliana
  Sequence located in RPL19. t 72-81 and may interact with
HSGYGKRNGT

SEQ ID NO: 673 - protein, 10 aa, Arabidopsis Thaliana
  Sequence located in GPA1 OR T1D16.6. at 296-305 and may
PVTSIAFSVS

SEQ ID NO: 674 - protein, 10 aa, Arabidopsis Thaliana
  Sequence located in RPL19. t 71-80 and may interact with
RHSGYGKRNG

SEQ ID NO: 675 - protein, 10 aa, Arabidopsis Thaliana
  Sequence located in GPA1 OR T1D16.6. at 136-145 and may
LSSTADKDGT

SEQ ID NO: 676 - protein, 10 aa, Arabidopsis Thaliana
  Sequence located in ASA1. a 362-371 and may interact with
QARGCILVAS

SEQ ID NO: 677 - protein, 10 aa, Arabidopsis Thaliana
  Sequence located in GBF1. a 176-185 and may interact with
ADASSQSTTG

SEQ ID NO: 678 - protein, 10 aa, Arabidopsis Thaliana
  Sequence located in GRP7. a 184-193 and may interact with
SGGGLGGGIG

SEQ ID NO: 679 - protein, 10 aa, Arabidopsis Thaliana
  Sequence located in GBF1. a 176-185 and may interact with
ADASSQSTTG

SEQ ID NO: 680 - protein, 10 aa, Arabidopsis Thaliana
  Sequence located in GRP7. a 126-135 and may interact with
SGGGLGGGIG

SEQ ID NO: 681 - protein, 10 aa, Arabidopsis Thaliana
  Sequence located in GBF1. a 10-19 and may interact with
FKTTKPTSSA

SEQ ID NO: 682 - protein, 10 aa, Arabidopsis Thaliana
  Sequence located in AGAA.3 R T10M13.8. at 105-114 and may
EFGGFGCATG

SEQ ID NO: 683 - protein, 10 aa, Arabidopsis Thaliana
  Sequence located in GBF3 OR T3F17.8. at 18-27 and may
NSSSASSSPS

SEQ ID NO: 684 - protein, 10 aa, Arabidopsis Thaliana
  Sequence located in GRP7. a 61-70 and may interact with
GGGAGGGGGI

SEQ ID NO: 685 - protein, 10 aa, Arabidopsis Thaliana
  Sequence located in GBF3 OR T3F17.8. at 167-176 and may
GGGVTRGKRG

SEQ ID NO: 686 - protein, 10 aa, Arabidopsis Thaliana
  Sequence located in T06B20. 4 OR T09D09.20. at 100-109 and
TAPYSPSLSA

SEQ ID NO: 687 - protein, 10 aa, Arabidopsis Thaliana
  Sequence located in T09D09. 4. at 72-81 and may interact with
IGDGQDILVR

SEQ ID NO: 688 - protein, 10 aa, Arabidopsis Thaliana
  Sequence located in PRXEA. t 286-295 and may interact with
DTIPLVNQYS

SEQ ID NO: 689 - protein, 10 aa, Arabidopsis Thaliana
  Sequence located in T09D09. 4. at 128-137 and may interact with
GIGLGHKLRA

SEQ ID NO: 690 - protein, 10 aa, Arabidopsis Thaliana
  Sequence located in PTR2-A R PTR2. at 408-417 and may
AYAKSVLKTS

SEQ ID NO: 691 - protein, 10 aa, Arabidopsis Thaliana
  Sequence located in T09D09. 4. at 84-93 and may interact with
SECLTGDIFG

SEQ ID NO: 692 - protein, 10 aa, Arabidopsis Thaliana
  Sequence located in T29E15. 3. at 295-304 and may interact with
AEDVTGEALA

SEQ ID NO: 693 - protein, 10 aa, Arabidopsis Thaliana
  Sequence located in Unknown at 28-37 and may interact with
FSFSRCFSTV

SEQ ID NO: 694 - protein, 10 aa, Arabidopsis Thaliana
  Sequence located in RPS11. t 24-33 and may interact with
KGKRPGKGGN

SEQ ID NO: 695 - protein, 10 aa, Arabidopsis Thaliana
  Sequence located in Unknown at 27-36 and may interact with
PFSFSRCFST

SEQ ID NO: 696 - protein, 10 aa, Arabidopsis Thaliana
  Sequence located in RPS11. t 23-32 and may interact with
GKGKRPGKGG

SEQ ID NO: 697 - protein, 10 aa, Arabidopsis Thaliana
  Sequence located in GLX2-2. at 109-118 and may interact with
TPCHTKGHIS

SEQ ID NO: 698 - protein, 10 aa, Arabidopsis Thaliana
  Sequence located in CYP73A5 OR CYP73 OR T06B20.16. at
RYVPFGVGRR

SEQ ID NO: 699 - protein, 10 aa, Arabidopsis Thaliana
  Sequence located in GRP7. a 252-261 and may interact with
AGGGFGGGAG

SEQ ID NO: 700 - protein, 10 aa, Arabidopsis Thaliana
  Sequence located in TOP1. a 355-364 and may interact with
STSSKSSPSS

SEQ ID NO: 701 - protein, 10 aa, Arabidopsis Thaliana
  Sequence located in GRP7. a 236-245 and may interact with
TGGGFGGGAG

SEQ ID NO: 702 - protein, 10 aa, Arabidopsis Thaliana
  Sequence located in TOP1. a 355-364 and may interact with
STSSKSSPSS

SEQ ID NO: 703 - protein, 10 aa, Arabidopsis Thaliana
  Sequence located in GRP7. a 224-233 and may interact with
AGGGLGGGAG

SEQ ID NO: 704 - protein, 10 aa, Arabidopsis Thaliana
  Sequence located in TOP1. a 355-364 and may interact with
STSSKSSPSS

SEQ ID NO: 705 - protein, 10 aa, Arabidopsis Thaliana
  Sequence located in GRP7. a 78-87 and may interact with
AGGGLGGGAG

SEQ ID NO: 706 - protein, 10 aa, Arabidopsis Thaliana
  Sequence located in TOP1. a 355-364 and may interact with
STSSKSSPSS

SEQ ID NO: 707 - protein, 10 aa, Arabidopsis Thaliana
  Sequence located in GRP7. a 56-65 and may interact with
AGGGLGGGAG

SEQ ID NO: 708 - protein, 10 aa, Arabidopsis Thaliana
  Sequence located in TOP1. a 355-364 and may interact with
STSSKSSPSS

SEQ ID NO: 709 - protein, 10 aa, Arabidopsis Thaliana
  Sequence located in GRP7. a 44-53 and may interact with
AGGGFGGGAG

SEQ ID NO: 710 - protein, 10 aa, Arabidopsis Thaliana
  Sequence located in TOP1. a 355-364 and may interact with
STSSKSSPSS

SEQ ID NO: 711 - protein, 10 aa, Arabidopsis Thaliana
  Sequence located in GSH1. a 512-521 and may interact with
KRRSRVRRAA

SEQ ID NO: 712 - protein, 10 aa, Arabidopsis Thaliana
  Sequence located in VALRS. t 17-26 and may interact with
CSATHSRSSF

SEQ ID NO: 713 - protein, 10 aa, Arabidopsis Thaliana
  Sequence located in ERD11 O GST1. at 124-133 and may
SSDTAGGVGG

SEQ ID NO: 714 - protein, 10 aa, Arabidopsis Thaliana
  Sequence located in ERD11 O GST1. at 124-133 and may
SSDTAGGVGG

SEQ ID NO: 715 - protein, 10 aa, Arabidopsis Thaliana
  Sequence located in ERD11 O GST1. at 13-22 and may interact
SSSPSLQFLC

SEQ ID NO: 716 - protein, 10 aa, Arabidopsis Thaliana
  Sequence located in Unknown at 103-112 and may interact with
RRARTKLKQT

SEQ ID NO: 717 - protein, 10 aa, Arabidopsis Thaliana
  Sequence located in ERD11 O GST1. at 13-22 and may interact
SSSPSLQFLC

SEQ ID NO: 718 - protein, 10 aa, Arabidopsis Thaliana
  Sequence located in HAT4 OR ATHB-2. at 177-186 and may
RRARTKLKQT

SEQ ID NO: 719 - protein, 10 aa, Arabidopsis Thaliana
  Sequence located in ERD11 O GST1. at 8-17 and may interact
TSTISSSSPS

SEQ ID NO: 720 - protein, 10 aa, Arabidopsis Thaliana
  Sequence located in HAT9. a 226-235 and may interact with
GGGGGGNGGG

SEQ ID NO: 721 - protein, 10 aa, Arabidopsis Thaliana
  Sequence located in ERD11 O GST1. at 13-22 and may interact
SSSPSLQFLC

SEQ ID NO: 722 - protein, 10 aa, Arabidopsis Thaliana
  Sequence located in HAT9. a 161-170 and may interact with
RRARTKLKQT

SEQ ID NO: 723 - protein, 10 aa, Arabidopsis Thaliana
  Sequence located in ERD11 O GST1. at 8-17 and may interact
TSTISSSSPS

SEQ ID NO: 724 - protein, 10 aa, Arabidopsis Thaliana
  Sequence located in HAT9. a 229-238 and may interact with
GGGNGGGGGG

SEQ ID NO: 725 - protein, 10 aa, Arabidopsis Thaliana
  Sequence located in ERD11 O GST1. at 92-101 and may
TIGAGSGGVR

SEQ ID NO: 726 - protein, 10 aa, Arabidopsis Thaliana
  Sequence located in GL2. at 29-38 and may interact with
RNASSGSTNP

SEQ ID NO: 727 - protein, 10 aa, Arabidopsis Thaliana
  Sequence located in ERD11 O GST1. at 13-22 and may interact
SSSPSLQFLC

SEQ ID NO: 728 - protein, 10 aa, Arabidopsis Thaliana
  Sequence located in HSF1. a 67-76 and may interact with
RRARTKLKQT

SEQ ID NO: 729 - protein, 10 aa, Arabidopsis Thaliana
  Sequence located in ERD11 O GST1. at 124-133 and may
SSDTAGGVGG

SEQ ID NO: 730 - protein, 10 aa, Arabidopsis Thaliana
  Sequence located in Unknown at 10-19 and may interact with
TTIRSSSYPT

SEQ ID NO: 731 - protein, 10 aa, Arabidopsis Thaliana
  Sequence located in ERD11 O GST1. at 93-102 and may
IGAGSGGVRA

SEQ ID NO: 732 - protein, 10 aa, Arabidopsis Thaliana
  Sequence located in RPB205 R RPII OR RPB1. at 1805-1814
SPYSSGASPD

SEQ ID NO: 733 - protein, 10 aa, Arabidopsis Thaliana
  Sequence located in ERD11 O GST1. at 93-102 and may
IGAGSGGVRA

SEQ ID NO: 734 - protein, 10 aa, Arabidopsis Thaliana
  Sequence located in RPB205 R RPII OR RPB1. at 1786-1795
SPYSSGASPD

SEQ ID NO: 735 - protein, 10 aa, Arabidopsis Thaliana
  Sequence located in ERD13 O F7F1.8. at 99-108 and may
DVEATSYHPP

SEQ ID NO: 736 - protein, 10 aa, Arabidopsis Thaliana
  Sequence located in ND6 OR AD6. at 188-197 and may interact
IDFRRTIMRR

SEQ ID NO: 737 - protein, 10 aa, Arabidopsis Thaliana
  Sequence located in 103-1A. at 109-118 and may interact with
SSFDPPASSL

SEQ ID NO: 738 - protein, 10 aa, Arabidopsis Thaliana
  Sequence located in Unknown at 527-536 and may interact with
AGEVRWGGAK

SEQ ID NO: 739 - protein, 10 aa, Arabidopsis Thaliana
  Sequence located in Unknown at 8-17 and may interact with
RKSTGGKAPR

SEQ ID NO: 740 - protein, 10 aa, Arabidopsis Thaliana
  Sequence located in PYRB. a 6-15 and may interact with
SLTSATLCGA

SEQ ID NO: 741 - protein, 10 aa, Arabidopsis Thaliana
  Sequence located in Unknown at 7-16 and may interact with
ARKSTGGKAP

SEQ ID NO: 742 - protein, 10 aa, Arabidopsis Thaliana
  Sequence located in PYRB. a 5-14 and may interact with
SSLTSATLCG

SEQ ID NO: 743 - protein, 10 aa, Arabidopsis Thaliana
  Sequence located in Unknown at 8-17 and may interact with
RKSTGGKAPR

SEQ ID NO: 744 - protein, 10 aa, Arabidopsis Thaliana
  Sequence located in PYRB. a 6-15 and may interact with
SLTSATLCGA

SEQ ID NO: 745 - protein, 10 aa, Arabidopsis Thaliana
  Sequence located in Unknown at 7-16 and may interact with
ARKSTGGKAP

SEQ ID NO: 746 - protein, 10 aa, Arabidopsis Thaliana
  Sequence located in PYRB. a 5-14 and may interact with
SSLTSATLCG

SEQ ID NO: 747 - protein, 10 aa, Arabidopsis Thaliana
  Sequence located in HAT4 OR ATHB-2. at 272-281 and may
SHGLTFDALR

SEQ ID NO: 748 - protein, 10 aa, Arabidopsis Thaliana
  Sequence located in S1FA. a 70-79 and may interact with
TVTKCEIGQS

SEQ ID NO: 749 - protein, 10 aa, Arabidopsis Thaliana
  Sequence located in HAT7 OR ATHB-3. at 196-205 and may
RSATATTAST

SEQ ID NO: 750 - protein, 10 aa, Arabidopsis Thaliana
  Sequence located in HAT9. a 233-242 and may interact with
GGGGGGSGAT

SEQ ID NO: 751 - protein, 10 aa, Arabidopsis Thaliana
  Sequence located in HAT7 OR ATHB-3. at 196-205 and may
RSATATTAST

SEQ ID NO: 752 - protein, 10 aa, Arabidopsis Thaliana
  Sequence located in RLK5 OR F21O9.180. at 43-52 and may
SGGGGGGGAP

SEQ ID NO: 753 - protein, 10 aa, Arabidopsis Thaliana
  Sequence located in HAT7 OR ATHB-3. at 196-205 and may
RSATATTAST

SEQ ID NO: 754 - protein, 10 aa, Arabidopsis Thaliana
  Sequence located in RLK5 OR F21O9.180. at 41-50 and may
ATSGGGGGGG

SEQ ID NO: 755 - protein, 10 aa, Arabidopsis Thaliana
  Sequence located in HEMC. a 42-51 and may interact with
YGYCGTTADY

SEQ ID NO: 756 - protein, 10 aa, Arabidopsis Thaliana
  Sequence located in F21J9.1 . at 329-338 and may interact with
IAIATGGRIV

SEQ ID NO: 757 - protein, 10 aa, Arabidopsis Thaliana
  Sequence located in HEMC. a 41-50 and may interact with
QYGYCGTTAD

SEQ ID NO: 758 - protein, 10 aa, Arabidopsis Thaliana
  Sequence located in F21J9.1 . at 328-337 and may interact with
LIAIATGGRI

SEQ ID NO: 759 - protein, 10 aa, Arabidopsis Thaliana
  Sequence located in GL2. at 99-108 and may interact with
KRKKYHRHTT

SEQ ID NO: 760 - protein, 10 aa, Arabidopsis Thaliana
  Sequence located in RD19A. t 13-22 and may interact with
LSFFIVSVSS

SEQ ID NO: 761 - protein, 10 aa, Arabidopsis Thaliana
  Sequence located in K1. at 28-237 and may interact with
FSKKKKKGKL

SEQ ID NO: 762 - protein, 10 aa, Arabidopsis Thaliana
  Sequence located in PPX1. a 89-98 and may interact with
ETFLLLLALK

SEQ ID NO: 763 - protein, 10 aa, Arabidopsis Thaliana
  Sequence located in HEMA1 O HEMA. at 80-89 and may
SPSSAAAAAA

SEQ ID NO: 764 - protein, 10 aa, Arabidopsis Thaliana
  Sequence located in T01O24. 6. at 67-76 and may interact with
GRCRSGGAGA

SEQ ID NO: 765 - protein, 10 aa, Arabidopsis Thaliana
  Sequence located in HEMA1 O HEMA. at 81-90 and may
PSSAAAAAAA

SEQ ID NO: 766 - protein, 10 aa, Arabidopsis Thaliana
  Sequence located in T01O24. 6. at 66-75 and may interact with
GGRCRSGGAG

SEQ ID NO: 767 - protein, 10 aa, Arabidopsis Thaliana
  Sequence located in HEMA1 O HEMA. at 140-149 and may
NDGGGATAAD

SEQ ID NO: 768 - protein, 10 aa, Arabidopsis Thaliana
  Sequence located in LTP1. a 48-57 and may interact with
VIPPACCSGV

SEQ ID NO: 769 - protein, 10 aa, Arabidopsis Thaliana
  Sequence located in HEMA1 O HEMA. at 66-75 and may
HTATANTTTA

SEQ ID NO: 770 - protein, 10 aa, Arabidopsis Thaliana
  Sequence located in RBP31 O RNPT. at 34-43 and may interact
SSSRIRSSSV

SEQ ID NO: 771 - protein, 10 aa, Arabidopsis Thaliana
  Sequence located in HEMA1 O HEMA. at 81-90 and may
PSSAAAAAAA

SEQ ID NO: 772 - protein, 10 aa, Arabidopsis Thaliana
  Sequence located in Unknown at 83-92 and may interact with
GGGSRRSSSS

SEQ ID NO: 773 - protein, 10 aa, Arabidopsis Thaliana
  Sequence located in Unknown at 52-61 and may interact with
TAATAAATSS

SEQ ID NO: 774 - protein, 10 aa, Arabidopsis Thaliana
  Sequence located in T01O24. 6. at 66-75 and may interact with
GGRCRSGGAG

SEQ ID NO: 775 - protein, 10 aa, Arabidopsis Thaliana
  Sequence located in Unknown at 74-83 and may interact with
SRSSSFLQRG

SEQ ID NO: 776 - protein, 10 aa, Arabidopsis Thaliana
  Sequence located in PME2. a 75-84 and may interact with
AATGGKELTS

SEQ ID NO: 777 - protein, 10 aa, Arabidopsis Thaliana
  Sequence located in Unknown at 52-61 and may interact with
TAATAAATSS

SEQ ID NO: 778 - protein, 10 aa, Arabidopsis Thaliana
  Sequence located in RLK5 OR F21O9.180. at 41-50 and may
ATSGGGGGGG

SEQ ID NO: 779 - protein, 10 aa, Arabidopsis Thaliana
  Sequence located in Unknown at 75-84 and may interact with
RSSSFLQRGN

SEQ ID NO: 780 - protein, 10 aa, Arabidopsis Thaliana
  Sequence located in RLK5 OR F21O9.180. at 19-28 and may
AAGREKLASV

SEQ ID NO: 781 - protein, 10 aa, Arabidopsis Thaliana
  Sequence located in Unknown at 51-60 and may interact with
HTAATAAATS

SEQ ID NO: 782 - protein, 10 aa, Arabidopsis Thaliana
  Sequence located in Unknown at 84-93 and may interact with
GGSRRSSSSM

SEQ ID NO: 783 - protein, 10 aa, Arabidopsis Thaliana
  Sequence located in Unknown at 52-61 and may interact with
TAATAAATSS

SEQ ID NO: 784 - protein, 10 aa, Arabidopsis Thaliana
  Sequence located in Unknown at 83-92 and may interact with
GGGSRRSSSS

SEQ ID NO: 785 - protein, 10 aa, Arabidopsis Thaliana
  Sequence located in Unknown at 384-393 and may interact with
NAGENSGRDR

SEQ ID NO: 786 - protein, 10 aa, Arabidopsis Thaliana
  Sequence located in Unknown at 253-262 and may interact with
VGPFIGAAIA

SEQ ID NO: 787 - protein, 10 aa, Arabidopsis Thaliana
  Sequence located in HMG1. a 576-585 and may interact with
RSSRDISGAT

SEQ ID NO: 788 - protein, 10 aa, Arabidopsis Thaliana
  Sequence located in HMG1. a 576-585 and may interact with
RSSRDISGAT

SEQ ID NO: 789 - protein, 10 aa, Arabidopsis Thaliana
  Sequence located in HMG2 OR F5J6.13. at 520-529 and may
VAGSVLAGEL

SEQ ID NO: 790 - protein, 10 aa, Arabidopsis Thaliana
  Sequence located in TOP1. a 231-240 and may interact with
NSSADQSSLK

SEQ ID NO: 791 - protein, 10 aa, Arabidopsis Thaliana
  Sequence located in HEM15. t 63-72 and may interact with
QARSVTGDCS

SEQ ID NO: 792 - protein, 10 aa, Arabidopsis Thaliana
  Sequence located in RPOMT. t 893-902 and may interact with
TAVACNRAGL

SEQ ID NO: 793 - protein, 10 aa, Arabidopsis Thaliana
  Sequence located in T06B20. 4 OR T09D09.20. at 199-208 and
KSEFLPGFER

SEQ ID NO: 794 - protein, 10 aa, Arabidopsis Thaliana
  Sequence located in T09D09. 3. at 217-226 and may interact with
LGFKERSELS

SEQ ID NO: 795 - protein, 10 aa, Arabidopsis Thaliana
  Sequence located in T06B20. 4 OR T09D09.20. at 320-329 and
LREMRKRSSI

SEQ ID NO: 796 - protein, 10 aa, Arabidopsis Thaliana
  Sequence located in RPB205 R RPII OR RPB1. at 820-829 and
DGRTLPHFTK

SEQ ID NO: 797 - protein, 10 aa, Arabidopsis Thaliana
  Sequence located in T06B20. 4 OR T09D09.20. at 320-329 and
LREMRKRSSI

SEQ ID NO: 798 - protein, 10 aa, Arabidopsis Thaliana
  Sequence located in RPB205 R RPII OR RPB1. at 794-803 and
DGRTLPHFTK

SEQ ID NO: 799 - protein, 10 aa, Arabidopsis Thaliana
  Sequence located in HSP23.6 at 9-18 and may interact with
RLLSSSIAPR

SEQ ID NO: 800 - protein, 10 aa, Arabidopsis Thaliana
  Sequence located in DGK1. a 166-175 and may interact with
SEKTRGNGWS

SEQ ID NO: 801 - protein, 10 aa, Arabidopsis Thaliana
  Sequence located in HSP23.6 at 54-63 and may interact with
RRSVPRRRGD

SEQ ID NO: 802 - protein, 10 aa, Arabidopsis Thaliana
  Sequence located in Unknown at 118-127 and may interact with
IATTSGYATP

SEQ ID NO: 803 - protein, 10 aa, Arabidopsis Thaliana
  Sequence located in HSP23.6 at 127-136 and may interact with
LSREDVKLAL

SEQ ID NO: 804 - protein, 10 aa, Arabidopsis Thaliana
  Sequence located in Unknown at 147-156 and may interact with
EGPLIYLKRE

SEQ ID NO: 805 - protein, 10 aa, Arabidopsis Thaliana
  Sequence located in Unknown at 252-261 and may interact with
GGGEDASAVT

SEQ ID NO: 806 - protein, 10 aa, Arabidopsis Thaliana
  Sequence located in S1FA. a 7-16 and may interact with
CYSRGILPPS

SEQ ID NO: 807 - protein, 10 aa, Arabidopsis Thaliana
  Sequence located in Unknown at 281-290 and may interact with
IPDSITIGGT

SEQ ID NO: 808 - protein, 10 aa, Arabidopsis Thaliana
  Sequence located in Unknown at 246-255 and may interact with
DRIGNRDAAS

SEQ ID NO: 809 - protein, 10 aa, Arabidopsis Thaliana
  Sequence located in Unknown at 280-289 and may interact with
VIPDSITIGG

SEQ ID NO: 810 - protein, 10 aa, Arabidopsis Thaliana
  Sequence located in Unknown at 245-254 and may interact with
YDRIGNRDAA

SEQ ID NO: 811 - protein, 10 aa, Arabidopsis Thaliana
  Sequence located in ILL2. a 282-291 and may interact with
IPDSITIGGT

SEQ ID NO: 812 - protein, 10 aa, Arabidopsis Thaliana
  Sequence located in Unknown at 246-255 and may interact with
DRIGNRDAAS

SEQ ID NO: 813 - protein, 10 aa, Arabidopsis Thaliana
  Sequence located in ILL2. a 281-290 and may interact with
VIPDSITIGG

SEQ ID NO: 814 - protein, 10 aa, Arabidopsis Thaliana
  Sequence located in Unknown at 245-254 and may interact with
YDRIGNRDAA

SEQ ID NO: 815 - protein, 10 aa, Arabidopsis Thaliana
  Sequence located in IMPDH. t 198-207 and may interact with
VVLERDGETV

SEQ ID NO: 816 - protein, 10 aa, Arabidopsis Thaliana
  Sequence located in Unknown at 100-109 and may interact with
YGLTVPFQNY

SEQ ID NO: 817 - protein, 10 aa, Arabidopsis Thaliana
  Sequence located in Unknown at 51-60 and may interact with
APTVFNCAVE

SEQ ID NO: 818 - protein, 10 aa, Arabidopsis Thaliana
  Sequence located in Unknown at 435-444 and may interact with
LNSAIKHRRG

SEQ ID NO: 819 - protein, 10 aa, Arabidopsis Thaliana
  Sequence located in T01O24. 6. at 80-89 and may interact with
ICLCDYCSDK

SEQ ID NO: 820 - protein, 10 aa, Arabidopsis Thaliana
  Sequence located in Unknown at 860-869 and may interact with
DREGVVTGVF

SEQ ID NO: 821 - protein, 10 aa, Arabidopsis Thaliana
  Sequence located in T01O24. 6. at 64-73 and may interact with
AKGGRCRSGG

SEQ ID NO: 822 - protein, 10 aa, Arabidopsis Thaliana
  Sequence located in RPOPT. t 3-12 and may interact with
SAAAASPSLS

SEQ ID NO: 823 - protein, 10 aa, Arabidopsis Thaliana
  Sequence located in KATB. a 462-471 and may interact with
ISQLVQSALD

SEQ ID NO: 824 - protein, 10 aa, Arabidopsis Thaliana
  Sequence located in PRC8. a 118-127 and may interact with
IKCRLHELAN

SEQ ID NO: 825 - protein, 10 aa, Arabidopsis Thaliana
  Sequence located in KATB. a 727-736 and may interact with
AARVNACEIG

SEQ ID NO: 826 - protein, 10 aa, Arabidopsis Thaliana
  Sequence located in Unknown at 89-98 and may interact with
SRTDVSTLDS

SEQ ID NO: 827 - protein, 10 aa, Arabidopsis Thaliana
  Sequence located in DGK1. a 332-341 and may interact with
TGAGGGGCVL

SEQ ID NO: 828 - protein, 10 aa, Arabidopsis Thaliana
  Sequence located in KIN2 OR COR6.6. at 30-39 and may interact
KDAAAAAGAS

SEQ ID NO: 829 - protein, 10 aa, Arabidopsis Thaliana
  Sequence located in DGK1. a 328-337 and may interact with
VSKLTGAGGG

SEQ ID NO: 830 - protein, 10 aa, Arabidopsis Thaliana
  Sequence located in Unknown at 17-26 and may interact with
APSSSSQLTH

SEQ ID NO: 831 - protein, 10 aa, Arabidopsis Thaliana
  Sequence located in DGK1. a 329-338 and may interact with
SKLTGAGGGG

SEQ ID NO: 832 - protein, 10 aa, Arabidopsis Thaliana
  Sequence located in Unknown at 16-25 and may interact with
SAPSSSSQLT

SEQ ID NO: 833 - protein, 10 aa, Arabidopsis Thaliana
  Sequence located in Unknown at 32-41 and may interact with
AAAGAGAGAQ

SEQ ID NO: 834 - protein, 10 aa, Arabidopsis Thaliana
  Sequence located in RPOPT. t 58-67 and may interact with
SSSSSSSTSL

SEQ ID NO: 835 - protein, 10 aa, Arabidopsis Thaliana
  Sequence located in Unknown at 31-40 and may interact with
DAAAGAGAGA

SEQ ID NO: 836 - protein, 10 aa, Arabidopsis Thaliana
  Sequence located in Unknown at 37-46 and may interact with
SSSSSTSSSV

SEQ ID NO: 837 - protein, 10 aa, Arabidopsis Thaliana
  Sequence located in KIN2 OR COR6.6. at 32-41 and may interact
AAAAAGASAQ

SEQ ID NO: 838 - protein, 10 aa, Arabidopsis Thaliana
  Sequence located in RPOPT. t 58-67 and may interact with
SSSSSSSTSL

SEQ ID NO: 839 - protein, 10 aa, Arabidopsis Thaliana
  Sequence located in Unknown at 425-434 and may interact with
SNEAPARHSL

SEQ ID NO: 840 - protein, 10 aa, Arabidopsis Thaliana
  Sequence located in RPL34. t 80-89 and may interact with
GVLSGSAVRE

SEQ ID NO: 841 - protein, 10 aa, Arabidopsis Thaliana
  Sequence located in Unknown at 424-433 and may interact with
CSNEAPARHS

SEQ ID NO: 842 - protein, 10 aa, Arabidopsis Thaliana
  Sequence located in RPL34. t 79-88 and may interact with
GGVLSGSAVR

SEQ ID NO: 843 - protein, 10 aa, Arabidopsis Thaliana
  Sequence located in Unknown at 295-304 and may interact with
PRPTRAEATD

SEQ ID NO: 844 - protein, 10 aa, Arabidopsis Thaliana
  Sequence located in VPS41. t 314-323 and may interact with
RPGRSRLRCI

SEQ ID NO: 845 - protein, 10 aa, Arabidopsis Thaliana
  Sequence located in LFY. at 341-350 and may interact with
SPNLRCAALD

SEQ ID NO: 846 - protein, 10 aa, Arabidopsis Thaliana
  Sequence located in ABI2. a 278-287 and may interact with
ARIEAAGGKV

SEQ ID NO: 847 - protein, 10 aa, Arabidopsis Thaliana
  Sequence located in Unknown at 89-98 and may interact with
GGVTGKPKSL

SEQ ID NO: 848 - protein, 10 aa, Arabidopsis Thaliana
  Sequence located in F4P9.44 OR T01B08.26. at 202-211 and may
ERFRFARDTS

SEQ ID NO: 849 - protein, 10 aa, Arabidopsis Thaliana
  Sequence located in SAM1. a 135-144 and may interact with
RDALESLLAK

SEQ ID NO: 850 - protein, 10 aa, Arabidopsis Thaliana
  Sequence located in RPS17. t 35-44 and may interact with
SICKFAKKGL

SEQ ID NO: 851 - protein, 10 aa, Arabidopsis Thaliana
  Sequence located in MPK4. a 173-182 and may interact with
SNLLLNSNCD

SEQ ID NO: 852 - protein, 10 aa, Arabidopsis Thaliana
  Sequence located in RPOPT. t 275-284 and may interact with
IGIAIEQEVR

SEQ ID NO: 853 - protein, 10 aa, Arabidopsis Thaliana
  Sequence located in MPK6 OR F18O19.10. at 54-63 and may
TNERVAIKKI

SEQ ID NO: 854 - protein, 10 aa, Arabidopsis Thaliana
  Sequence located in T20K9.8 at 115-124 and may interact with
YFLNRDSLVC

SEQ ID NO: 855 - protein, 10 aa, Arabidopsis Thaliana
  Sequence located in MS2. at 45-54 and may interact with
VGGGGDGGRN

SEQ ID NO: 856 - protein, 10 aa, Arabidopsis Thaliana
  Sequence located in RD19A. t 359-368 and may interact with
VAATVSTTAH

SEQ ID NO: 857 - protein, 10 aa, Arabidopsis Thaliana
  Sequence located in MS2. at 46-55 and may interact with
GGGGDGGRNS

SEQ ID NO: 858 - protein, 10 aa, Arabidopsis Thaliana
  Sequence located in RD19A. t 358-367 and may interact with
TVAATVSTTA

SEQ ID NO: 859 - protein, 10 aa, Arabidopsis Thaliana
  Sequence located in MSH2. a 624-633 and may interact with
SDAGDIVLEG

SEQ ID NO: 860 - protein, 10 aa, Arabidopsis Thaliana
  Sequence located in PAB5. a 654-663 and may interact with
AVSSVDDQFA

SEQ ID NO: 861 - protein, 10 aa, Arabidopsis Thaliana
  Sequence located in MSH2. a 623-632 and may interact with
SSDAGDIVLE

SEQ ID NO: 862 - protein, 10 aa, Arabidopsis Thaliana
  Sequence located in PAB5. a 653-662 and may interact with
AAVSSVDDQF

SEQ ID NO: 863 - protein, 10 aa, Arabidopsis Thaliana
  Sequence located in AGAA.3 R T10M13.8. at 367-376 and may
LFIHGGHTSK

SEQ ID NO: 864 - protein, 10 aa, Arabidopsis Thaliana
  Sequence located in ARP1 OR F1I21.L3. at 305-314 and may
EKDVTPMGGF

SEQ ID NO: 865 - protein, 10 aa, Arabidopsis Thaliana
  Sequence located in MSI2. a 319-328 and may interact with
TVLASSGEDR

SEQ ID NO: 866 - protein, 10 aa, Arabidopsis Thaliana
  Sequence located in KN OR F 2O13.4. at 470-479 and may
SDKSGTPLVA

SEQ ID NO: 867 - protein, 10 aa, Arabidopsis Thaliana
  Sequence located in MSI4. a 77-86 and may interact with
IANCEVVKPR

SEQ ID NO: 868 - protein, 10 aa, Arabidopsis Thaliana
  Sequence located in RNS2 OR T5I7.8. at 76-85 and may interact
SRFDDLRVSD

SEQ ID NO: 869 - protein, 10 aa, Arabidopsis Thaliana
  Sequence located in MSI4. a 78-87 and may interact with
ANCEVVKPRV

SEQ ID NO: 870 - protein, 10 aa, Arabidopsis Thaliana
  Sequence located in RNS2 OR T5I7.8. at 75-84 and may interact
DSRFDDLRVS

SEQ ID NO: 871 - protein, 10 aa, Arabidopsis Thaliana
  Sequence located in MSI4. a 314-323 and may interact with
LFDRRKLTAN

SEQ ID NO: 872 - protein, 10 aa, Arabidopsis Thaliana
  Sequence located in Unknown at 829-838 and may interact with
KKVSALKSRV

SEQ ID NO: 873 - protein, 10 aa, Arabidopsis Thaliana
  Sequence located in MT2B. a 4-13 and may interact with
CGGSCGCGSA

SEQ ID NO: 874 - protein, 10 aa, Arabidopsis Thaliana
  Sequence located in VPS41. t 325-334 and may interact with
CTTAAAASAR

SEQ ID NO: 875 - protein, 10 aa, Arabidopsis Thaliana
  Sequence located in NDPK1. t 25-34 and may interact with
SLNPHCSPAR

SEQ ID NO: 876 - protein, 10 aa, Arabidopsis Thaliana
  Sequence located in Unknown at 203-212 and may interact with
TRWGRVGVKG

SEQ ID NO: 877 - protein, 10 aa, Arabidopsis Thaliana
  Sequence located in NDPK1. t 26-35 and may interact with
LNPHCSPARV

SEQ ID NO: 878 - protein, 10 aa, Arabidopsis Thaliana
  Sequence located in Unknown at 202-211 and may interact with
YTRWGRVGVK

SEQ ID NO: 879 - protein, 10 aa, Arabidopsis Thaliana
  Sequence located in NDPK1. t 27-36 and may interact with
NPHCSPARVN

SEQ ID NO: 880 - protein, 10 aa, Arabidopsis Thaliana
  Sequence located in Unknown at 201-210 and may interact with
VYTRWGRVGV

SEQ ID NO: 881 - protein, 10 aa, Arabidopsis Thaliana
  Sequence located in NDPK1. t 58-67 and may interact with
RRRLRASSSA

SEQ ID NO: 882 - protein, 10 aa, Arabidopsis Thaliana
  Sequence located in TOP2. a 18-27 and may interact with
APAKSRAAAG

SEQ ID NO: 883 - protein, 10 aa, Arabidopsis Thaliana
  Sequence located in LTP1. a 53-62 and may interact with
CCSGVKNLNS

SEQ ID NO: 884 - protein, 10 aa, Arabidopsis Thaliana
  Sequence located in PUR2. a 475-484 and may interact with
GVKVFHAGTG

SEQ ID NO: 885 - protein, 10 aa, Arabidopsis Thaliana
  Sequence located in LTP1. a 93-102 and may interact with
AAGIPKACGV

SEQ ID NO: 886 - protein, 10 aa, Arabidopsis Thaliana
  Sequence located in AQP.1. t 6-15 and may interact with
RRAYGFGRAD

SEQ ID NO: 887 - protein, 10 aa, Arabidopsis Thaliana
  Sequence located in LTP1. a 92-101 and may interact with
RAAGIPKACG

SEQ ID NO: 888 - protein, 10 aa, Arabidopsis Thaliana
  Sequence located in AQP.1. t 5-14 and may interact with
ARRAYGFGRA

SEQ ID NO: 889 - protein, 10 aa, Arabidopsis Thaliana
  Sequence located in LTP1. a 91-100 and may interact with
GRAAGIPKAC

SEQ ID NO: 890 - protein, 10 aa, Arabidopsis Thaliana
  Sequence located in AQP.1. t 4-13 and may interact with
SARRAYGFGR

SEQ ID NO: 891 - protein, 10 aa, Arabidopsis Thaliana
  Sequence located in LTP1. a 79-88 and may interact with
GAARALGSGL

SEQ ID NO: 892 - protein, 10 aa, Arabidopsis Thaliana
  Sequence located in Unknown at 156-165 and may interact with
SSRAGQSRAE

SEQ ID NO: 893 - protein, 10 aa, Arabidopsis Thaliana
  Sequence located in LTP1. a 78-87 and may interact with
QGAARALGSG

SEQ ID NO: 894 - protein, 10 aa, Arabidopsis Thaliana
  Sequence located in Unknown at 155-164 and may interact with
LSSRAGQSRA

SEQ ID NO: 895 - protein, 10 aa, Arabidopsis Thaliana
  Sequence located in NIT1. a 112-121 and may interact with
DVARKNHVYL

SEQ ID NO: 896 - protein, 10 aa, Arabidopsis Thaliana
  Sequence located in PAP1. a 348-357 and may interact with
KVDVVFAGHV

SEQ ID NO: 897 - protein, 10 aa, Arabidopsis Thaliana
  Sequence located in NIT1. a 113-122 and may interact with
VARKNHVYLV

SEQ ID NO: 898 - protein, 10 aa, Arabidopsis Thaliana
  Sequence located in PAP1. a 347-356 and may interact with
YKVDVVFAGH

SEQ ID NO: 899 - protein, 10 aa, Arabidopsis Thaliana
  Sequence located in Unknown at 352-361 and may interact with
VRASKRACYR

SEQ ID NO: 900 - protein, 10 aa, Arabidopsis Thaliana
  Sequence located in Unknown at 221-230 and may interact with
NPSGLSRGIT

SEQ ID NO: 901 - protein, 10 aa, Arabidopsis Thaliana
  Sequence located in ORC2 OR F13M22.6. at 37-46 and may
AAASPGGGRR

SEQ ID NO: 902 - protein, 10 aa, Arabidopsis Thaliana
  Sequence located in ORC2 OR F13M22.6. at 37-46 and may
AAASPGGGRR

SEQ ID NO: 903 - protein, 10 aa, Arabidopsis Thaliana
  Sequence located in ORC2 OR F13M22.6. at 37-46 and may
AAASPGGGRR

SEQ ID NO: 904 - protein, 10 aa, Arabidopsis Thaliana
  Sequence located in RLK5 OR F21O9.180. at 39-48 and may
ASATSGGGGG

SEQ ID NO: 905 - protein, 10 aa, Arabidopsis Thaliana
  Sequence located in ABI2. a 256-265 and may interact with
LCRGKTPLAL

SEQ ID NO: 906 - protein, 10 aa, Arabidopsis Thaliana
  Sequence located in Unknown at 971-980 and may interact with
KGKGGLASGQ

SEQ ID NO: 907 - protein, 10 aa, Arabidopsis Thaliana
  Sequence located in ABI2. a 257-266 and may interact with
CRGKTPLALS

SEQ ID NO: 908 - protein, 10 aa, Arabidopsis Thaliana
  Sequence located in Unknown at 970-979 and may interact with
GKGKGGLASG

SEQ ID NO: 909 - protein, 10 aa, Arabidopsis Thaliana
  Sequence located in ABI2. a 204-213 and may interact with
KWKKALFNSF

SEQ ID NO: 910 - protein, 10 aa, Arabidopsis Thaliana
  Sequence located in Unknown at 44-53 and may interact with
EGVKKSLFPL

SEQ ID NO: 911 - protein, 10 aa, Arabidopsis Thaliana
  Sequence located in P2. at 57-166 and may interact with
YVSAASGAVG

SEQ ID NO: 912 - protein, 10 aa, Arabidopsis Thaliana
  Sequence located in RPS6. a 3-12 and may interact with
TNGSASSAHV

SEQ ID NO: 913 - protein, 10 aa, Arabidopsis Thaliana
  Sequence located in P5CSA O P5CS1 OR T5I7.10. at 31-40 and
GRLALGRLGA

SEQ ID NO: 914 - protein, 10 aa, Arabidopsis Thaliana
  Sequence located in TOP2. a 1253-1262 and may interact with
ASESETTEAS

SEQ ID NO: 915 - protein, 10 aa, Arabidopsis Thaliana
  Sequence located in P5CSA O P5CS1 OR T5I7.10. at 31-40 and
GRLALGRLGA

SEQ ID NO: 916 - protein, 10 aa, Arabidopsis Thaliana
  Sequence located in TOP2. a 1218-1227 and may interact with
ASESETTEAS

SEQ ID NO: 917 - protein, 10 aa, Arabidopsis Thaliana
  Sequence located in P5CSB O P5CS2. at 13-22 and may interact
GSTAAVTSAP

SEQ ID NO: 918 - protein, 10 aa, Arabidopsis Thaliana
  Sequence located in PHYC. a 14-23 and may interact with
AASSGHRRSR

SEQ ID NO: 919 - protein, 10 aa, Arabidopsis Thaliana
  Sequence located in P5CSB O P5CS2. at 124-133 and may
GNIFIKNLDE

SEQ ID NO: 920 - protein, 10 aa, Arabidopsis Thaliana
  Sequence located in AHA1. a 339-348 and may interact with
SVDKNLVEVF

SEQ ID NO: 921 - protein, 10 aa, Arabidopsis Thaliana
  Sequence located in P5CSB O P5CS2. at 145-154 and may
SSFGNIVSCK

SEQ ID NO: 922 - protein, 10 aa, Arabidopsis Thaliana
  Sequence located in PTR2-A R PTR2. at 276-285 and may
TTESVYNAAL

SEQ ID NO: 923 - protein, 10 aa, Arabidopsis Thaliana
  Sequence located in P5CSB O P5CS2. at 522-531 and may
GGSGDVPPYD

SEQ ID NO: 924 - protein, 10 aa, Arabidopsis Thaliana
  Sequence located in PUR7. a 190-199 and may interact with
VVRGYVTGST

SEQ ID NO: 925 - protein, 10 aa, Arabidopsis Thaliana
  Sequence located in P5CSB O P5CS2. at 20-29 and may interact
SAPATSGGAT

SEQ ID NO: 926 - protein, 10 aa, Arabidopsis Thaliana
  Sequence located in Unknown at 83-92 and may interact with
GGGSRRSSSS

SEQ ID NO: 927 - protein, 10 aa, Arabidopsis Thaliana
  Sequence located in P5CSB O P5CS2. at 19-28 and may interact
TSAPATSGGA

SEQ ID NO: 928 - protein, 10 aa, Arabidopsis Thaliana
  Sequence located in Unknown at 82-91 and may interact with
SGGGSRRSSS

SEQ ID NO: 929 - protein, 10 aa, Arabidopsis Thaliana
  Sequence located in PAB5. a 131-140 and may interact with
KGNVFIKNLD

SEQ ID NO: 930 - protein, 10 aa, Arabidopsis Thaliana
  Sequence located in AHA1. a 338-347 and may interact with
LSVDKNLVEV

SEQ ID NO: 931 - protein, 10 aa, Arabidopsis Thaliana
  Sequence located in PAB5. a 646-655 and may interact with
LRRSADPAAV

SEQ ID NO: 932 - protein, 10 aa, Arabidopsis Thaliana
  Sequence located in Unknown at 316-325 and may interact with
ESPGSIGRRD

SEQ ID NO: 933 - protein, 10 aa, Arabidopsis Thaliana
  Sequence located in PAL1. a 674-683 and may interact with
REELGTELLT

SEQ ID NO: 934 - protein, 10 aa, Arabidopsis Thaliana
  Sequence located in PAL1. a 674-683 and may interact with
REELGTELLT

SEQ ID NO: 935 - protein, 10 aa, Arabidopsis Thaliana
  Sequence located in F16P2.5 at 27-36 and may interact with
NFDCSTTGFS

SEQ ID NO: 936 - protein, 10 aa, Arabidopsis Thaliana
  Sequence located in Unknown at 220-229 and may interact with
VEVGTRSTEA

SEQ ID NO: 937 - protein, 10 aa, Arabidopsis Thaliana
  Sequence located in PRXEA. t 94-103 and may interact with
KDAAPNANSA

SEQ ID NO: 938 - protein, 10 aa, Arabidopsis Thaliana
  Sequence located in RPS11. t 63-72 and may interact with
GTVSIRGRIL

SEQ ID NO: 939 - protein, 10 aa, Arabidopsis Thaliana
  Sequence located in PRXEA. t 95-104 and may interact with
DAAPNANSAR

SEQ ID NO: 940 - protein, 10 aa, Arabidopsis Thaliana
  Sequence located in RPS11. t 62-71 and may interact with
TGTVSIRGRI

SEQ ID NO: 941 - protein, 10 aa, Arabidopsis Thaliana
  Sequence located in PRXEA. t 210-219 and may interact with
TPRLYNFNGT

SEQ ID NO: 942 - protein, 10 aa, Arabidopsis Thaliana
  Sequence located in Unknown at 170-179 and may interact with
GTIEVVESGG

SEQ ID NO: 943 - protein, 10 aa, Arabidopsis Thaliana
  Sequence located in POSF21. at 15-24 and may interact with
PPPSPSGRCS

SEQ ID NO: 944 - protein, 10 aa, Arabidopsis Thaliana
  Sequence located in RLK5 OR F21O9.180. at 45-54 and may
GGGGGGAPAA

SEQ ID NO: 945 - protein, 10 aa, Arabidopsis Thaliana
  Sequence located in PHYC. a 441-450 and may interact with
LQVSEKRVLR

SEQ ID NO: 946 - protein, 10 aa, Arabidopsis Thaliana
  Sequence located in SAR1A O T10M13.9 OR AGAA.4. at 109-
ELDALLSDES

SEQ ID NO: 947 - protein, 10 aa, Arabidopsis Thaliana
  Sequence located in AHA1. a 331-340 and may interact with
GTLTLNKLSV

SEQ ID NO: 948 - protein, 10 aa, Arabidopsis Thaliana
  Sequence located in PYRB. a 115-124 and may interact with
SSQSEILKGY

SEQ ID NO: 949 - protein, 10 aa, Arabidopsis Thaliana
  Sequence located in AHA1. a 330-339 and may interact with
TGTLTLNKLS

SEQ ID NO: 950 - protein, 10 aa, Arabidopsis Thaliana
  Sequence located in PYRB. a 114-123 and may interact with
SSSQSEILKG

SEQ ID NO: 951 - protein, 10 aa, Arabidopsis Thaliana
  Sequence located in AHA1. a 626-635 and may interact with
VIISAVLTSR

SEQ ID NO: 952 - protein, 10 aa, Arabidopsis Thaliana
  Sequence located in TOP1. a 280-289 and may interact with
STREDGTDDD

SEQ ID NO: 953 - protein, 10 aa, Arabidopsis Thaliana
  Sequence located in PME1. a 374-383 and may interact with
GIINTAGAAK

SEQ ID NO: 954 - protein, 10 aa, Arabidopsis Thaliana
  Sequence located in Unknown at 17-26 and may interact with
TDDIGRASRL

SEQ ID NO: 955 - protein, 10 aa, Arabidopsis Thaliana
  Sequence located in PME2. a 465-474 and may interact with
QNCRIGGTSD

SEQ ID NO: 956 - protein, 10 aa, Arabidopsis Thaliana
  Sequence located in APP. at 427-436 and may interact with
IGGSTNTGIL

SEQ ID NO: 957 - protein, 10 aa, Arabidopsis Thaliana
  Sequence located in Unknown at 49-58 and may interact with
RKRNSSNDTY

SEQ ID NO: 958 - protein, 10 aa, Arabidopsis Thaliana
  Sequence located in T09D09. 3. at 73-82 and may interact with
ALPIATVVSV

SEQ ID NO: 959 - protein, 10 aa, Arabidopsis Thaliana
  Sequence located in F7G19.2 at 197-206 and may interact with
LFKCPSSRCR

SEQ ID NO: 960 - protein, 10 aa, Arabidopsis Thaliana
  Sequence located in RPS16. t 130-139 and may interact with
KKFGGRGARS

SEQ ID NO: 961 - protein, 10 aa, Arabidopsis Thaliana
  Sequence located in F7G19.2 at 555-564 and may interact with
RAYISAARRL

SEQ ID NO: 962 - protein, 10 aa, Arabidopsis Thaliana
  Sequence located in Unknown at 86-95 and may interact with
ESSSRTDVST

SEQ ID NO: 963 - protein, 10 aa, Arabidopsis Thaliana
  Sequence located in PTR2-A R PTR2. at 76-85 and may interact
SPSGEIALSS

SEQ ID NO: 964 - protein, 10 aa, Arabidopsis Thaliana
  Sequence located in RCA. at 430-439 and may interact with
GRGTFYGKGA

SEQ ID NO: 965 - protein, 10 aa, Arabidopsis Thaliana
  Sequence located in Unknown at 491-500 and may interact with
FASITGLEFA

SEQ ID NO: 966 - protein, 10 aa, Arabidopsis Thaliana
  Sequence located in RCA. at 192-201 and may interact with
GELESGNAGE

SEQ ID NO: 967 - protein, 10 aa, Arabidopsis Thaliana
  Sequence located in PUR2. a 302-311 and may interact with
EGEEASFFAL

SEQ ID NO: 968 - protein, 10 aa, Arabidopsis Thaliana
  Sequence located in PUR2. a 302-311 and may interact with
EGEEASFFAL

SEQ ID NO: 969 - protein, 10 aa, Arabidopsis Thaliana
  Sequence located in PUR2. a 460-469 and may interact with
GSIIKNLEEA

SEQ ID NO: 970 - protein, 10 aa, Arabidopsis Thaliana
  Sequence located in RPS15 O RIG OR F19P19.26. at 36-45 and
STDDLVKLFS

SEQ ID NO: 971 - protein, 10 aa, Arabidopsis Thaliana
  Sequence located in PUR2. a 334-343 and may interact with
TGPNTGGMGA

SEQ ID NO: 972 - protein, 10 aa, Arabidopsis Thaliana
  Sequence located in RPS15 O RIG OR F19P19.26. at 137-146
RPGVGATHSS

SEQ ID NO: 973 - protein, 10 aa, Arabidopsis Thaliana
  Sequence located in PUR2. a 15-24 and may interact with
FDSLPPCRRC

SEQ ID NO: 974 - protein, 10 aa, Arabidopsis Thaliana
  Sequence located in TOP2. a 23-32 and may interact with
RAAAGGKTIE

SEQ ID NO: 975 - protein, 10 aa, Arabidopsis Thaliana
  Sequence located in T09D09. 3. at 215-224 and may interact with
RVLGFKERSE

SEQ ID NO: 976 - protein, 10 aa, Arabidopsis Thaliana
  Sequence located in TBP1. a 142-151 and may interact with
SYEPELFPGL

SEQ ID NO: 977 - protein, 10 aa, Arabidopsis Thaliana
  Sequence located in ARAC2. t 2-11 and may interact with
STARFIKCVT

SEQ ID NO: 978 - protein, 10 aa, Arabidopsis Thaliana
  Sequence located in Unknown at 236-245 and may interact with
AGSSEDFTNS

SEQ ID NO: 979 - protein, 10 aa, Arabidopsis Thaliana
  Sequence located in RCA. at 6-15 and may interact with
STVGAINRAP

SEQ ID NO: 980 - protein, 10 aa, Arabidopsis Thaliana
  Sequence located in RPL34. t 71-80 and may interact with
RRTVNRAYGG

SEQ ID NO: 981 - protein, 10 aa, Arabidopsis Thaliana
  Sequence located in RD19A. t 53-62 and may interact with
LFKRKFGKVY

SEQ ID NO: 982 - protein, 10 aa, Arabidopsis Thaliana
  Sequence located in F4P9.44 OR T01B08.26. at 561-570 and may
VDLSEFTFKK

SEQ ID NO: 983 - protein, 10 aa, Arabidopsis Thaliana
  Sequence located in Unknown at 48-57 and may interact with
SPSTASTSRT

SEQ ID NO: 984 - protein, 10 aa, Arabidopsis Thaliana
  Sequence located in RLK5 OR F21O9.180. at 40-49 and may
SATSGGGGGG

SEQ ID NO: 985 - protein, 10 aa, Arabidopsis Thaliana
  Sequence located in Unknown at 211-220 and may interact with
PNVAVGEVNL

SEQ ID NO: 986 - protein, 10 aa, Arabidopsis Thaliana
  Sequence located in SAR1A O T10M13.9 OR AGAA.4. at 157-
KVNLTDSNVR

SEQ ID NO: 987 - protein, 10 aa, Arabidopsis Thaliana
  Sequence located in Unknown at 97-106 and may interact with
GTASTLTRGE

SEQ ID NO: 988 - protein, 10 aa, Arabidopsis Thaliana
  Sequence located in Unknown at 123-132 and may interact with
ARRGSQRTPL

SEQ ID NO: 989 - protein, 10 aa, Arabidopsis Thaliana
  Sequence located in Unknown at 97-106 and may interact with
GTASTLTRGE

SEQ ID NO: 990 - protein, 10 aa, Arabidopsis Thaliana
  Sequence located in Unknown at 40-49 and may interact with
PSSRSKSSSF

SEQ ID NO: 991 - protein, 10 aa, Arabidopsis Thaliana
  Sequence located in Unknown at 198-207 and may interact with
RWGLDPKEKA

SEQ ID NO: 992 - protein, 10 aa, Arabidopsis Thaliana
  Sequence located in A_TM021 04.12. at 430-439 and may
TPAKVRLFLC

SEQ ID NO: 993 - protein, 10 aa, Arabidopsis Thaliana
  Sequence located in RPL13 O BBC1. at 196-205 and may
RAAEAEKEEK

SEQ ID NO: 994 - protein, 10 aa, Arabidopsis Thaliana
  Sequence located in Unknown at 10-19 and may interact with
LLLLLSLSSP

SEQ ID NO: 995 - protein, 10 aa, Arabidopsis Thaliana
  Sequence located in RPL13 O BBC1. at 188-197 and may
RHAGARAKRA

SEQ ID NO: 996 - protein, 10 aa, Arabidopsis Thaliana
  Sequence located in Unknown at 405-414 and may interact with
GAFSTCTGVA

SEQ ID NO: 997 - protein, 10 aa, Arabidopsis Thaliana
  Sequence located in RPL19. t 6-15 and may interact with
RLRSSVLKCG

SEQ ID NO: 998 - protein, 10 aa, Arabidopsis Thaliana
  Sequence located in Unknown at 242-251 and may interact with
ATLKDATSEA

SEQ ID NO: 999 - protein, 10 aa, Arabidopsis Thaliana
  Sequence located in RPL21 O F21M12.8 OR F14J9.25. at 4-13
GHGVRARTRD

SEQ ID NO: 1000 - protein, 10 aa, Arabidopsis Thaliana
  Sequence located in RBP31 O RNPT. at 14-23 and may interact
AMADSSSSTI

SEQ ID NO: 1001 - protein, 10 aa, Arabidopsis Thaliana
  Sequence located in RPL23. t 3-12 and may interact with
KRGRGGTSGN

SEQ ID NO: 1002 - protein, 10 aa, Arabidopsis Thaliana
  Sequence located in Unknown at 32-41 and may interact with
LTPATTGASV

SEQ ID NO: 1003 - protein, 10 aa, Arabidopsis Thaliana
  Sequence located in RLK5 OR F21O9.180. at 26-35 and may
ASVPSGGDGG

SEQ ID NO: 1004 - protein, 10 aa, Arabidopsis Thaliana
  Sequence located in RLK5 OR F21O9.180. at 26-35 and may
ASVPSGGDGG

SEQ ID NO: 1005 - protein, 10 aa, Arabidopsis Thaliana
  Sequence located in RLK5 OR F21O9.180. at 39-48 and may
ASATSGGGGG

SEQ ID NO: 1006 - protein, 10 aa, Arabidopsis Thaliana
  Sequence located in RLK5 OR F21O9.180. at 39-48 and may
ASATSGGGGG

SEQ ID NO: 1007 - protein, 10 aa, Arabidopsis Thaliana
  Sequence located in RLK5 OR F21O9.180. at 42-51 and may
TSGGGGGGGA

SEQ ID NO: 1008 - protein, 10 aa, Arabidopsis Thaliana
  Sequence located in RPOPT. t 57-66 and may interact with
SSSSSSSSTS

SEQ ID NO: 1009 - protein, 10 aa, Arabidopsis Thaliana
  Sequence located in RLK5 OR F21O9.180. at 42-51 and may
TSGGGGGGGA

SEQ ID NO: 1010 - protein, 10 aa, Arabidopsis Thaliana
  Sequence located in RPOPT. t 54-63 and may interact with
SASSSSSSSS

SEQ ID NO: 1011 - protein, 10 aa, Arabidopsis Thaliana
  Sequence located in RLK5 OR F21O9.180. at 44-53 and may
GGGGGGGAPA

SEQ ID NO: 1012 - protein, 10 aa, Arabidopsis Thaliana
  Sequence located in RPS6. a 263-272 and may interact with
SAAPAAASWS

SEQ ID NO: 1013 - protein, 10 aa, Arabidopsis Thaliana
  Sequence located in RLK5 OR F21O9.180. at 42-51 and may
TSGGGGGGGA

SEQ ID NO: 1014 - protein, 10 aa, Arabidopsis Thaliana
  Sequence located in VPS41. t 325-334 and may interact with
CTTAAAASAR

SEQ ID NO: 1015 - protein, 10 aa, Arabidopsis Thaliana
  Sequence located in RLK5 OR F21O9.180. at 42-51 and may
TSGGGGGGGA

SEQ ID NO: 1016 - protein, 10 aa, Arabidopsis Thaliana
  Sequence located in VPS41. t 325-334 and may interact with
CTTAAAASAR

SEQ ID NO: 1017 - protein, 10 aa, Arabidopsis Thaliana
  Sequence located in RBP31 O RNPT. at 59-68 and may interact
VSLSLKTKTH

SEQ ID NO: 1018 - protein, 10 aa, Arabidopsis Thaliana
  Sequence located in RBP31 O RNPT. at 59-68 and may interact
VSLSLKTKTH

SEQ ID NO: 1019 - protein, 10 aa, Arabidopsis Thaliana
  Sequence located in RBP31 O RNPT. at 35-44 and may interact
SSRIRSSSVS

SEQ ID NO: 1020 - protein, 10 aa, Arabidopsis Thaliana
  Sequence located in T29M21. 5. at 200-209 and may interact
TDTAAADTAA

SEQ ID NO: 1021 - protein, 10 aa, Arabidopsis Thaliana
  Sequence located in RPB205 R RPII OR RPB1. at 617-626 and
IPKQINLLRY

SEQ ID NO: 1022 - protein, 10 aa, Arabidopsis Thaliana
  Sequence located in RPB205 R RPII OR RPB1. at 617-626 and
IPKQINLLRY

SEQ ID NO: 1023 - protein, 10 aa, Arabidopsis Thaliana
  Sequence located in RPB205 R RPII OR RPB1. at 617-626 and
IPKQINLLRY

SEQ ID NO: 1024 - protein, 10 aa, Arabidopsis Thaliana
  Sequence located in RPB205 R RPII OR RPB1. at 591-600 and
IPKQINLLRY

SEQ ID NO: 1025 - protein, 10 aa, Arabidopsis Thaliana
  Sequence located in RPB205 R RPII OR RPB1. at 1778-1787
PNYSPTSPSY

SEQ ID NO: 1026 - protein, 10 aa, Arabidopsis Thaliana
  Sequence located in SRP-54B at 404-413 and may interact with
IARGSGRIVR

SEQ ID NO: 1027 - protein, 10 aa, Arabidopsis Thaliana
  Sequence located in RPB205 R RPII OR RPB1. at 1779-1788
NYSPTSPSYS

SEQ ID NO: 1028 - protein, 10 aa, Arabidopsis Thaliana
  Sequence located in SRP-54B at 403-412 and may interact with
RIARGSGRIV

SEQ ID NO: 1029 - protein, 10 aa, Arabidopsis Thaliana
  Sequence located in RPB205 R RPII OR RPB1. at 591-600 and
IPKQINLLRY

SEQ ID NO: 1030 - protein, 10 aa, Arabidopsis Thaliana
  Sequence located in RPB205 R RPII OR RPB1. at 591-600 and
IPKQINLLRY

SEQ ID NO: 1031 - protein, 10 aa, Arabidopsis Thaliana
  Sequence located in RPB205 R RPII OR RPB1. at 1759-1768
PNYSPTSPSY

SEQ ID NO: 1032 - protein, 10 aa, Arabidopsis Thaliana
  Sequence located in SRP-54B at 404-413 and may interact with
IARGSGRIVR

SEQ ID NO: 1033 - protein, 10 aa, Arabidopsis Thaliana
  Sequence located in RPB205 R RPII OR RPB1. at 1760-1769
NYSPTSPSYS

SEQ ID NO: 1034 - protein, 10 aa, Arabidopsis Thaliana
  Sequence located in SRP-54B at 403-412 and may interact with
RIARGSGRIV

SEQ ID NO: 1035 - protein, 10 aa, Arabidopsis Thaliana
  Sequence located in RPOPT. t 378-387 and may interact with
FRHRFKTVTK

SEQ ID NO: 1036 - protein, 10 aa, Arabidopsis Thaliana
  Sequence located in RPOPT. t 378-387 and may interact with
FRHRFKTVTK

SEQ ID NO: 1037 - protein, 10 aa, Arabidopsis Thaliana
  Sequence located in (RPS18A OR PFL) AND RPS18B AND
GRLNKCGVIS

SEQ ID NO: 1038 - protein, 10 aa, Arabidopsis Thaliana
  Sequence located in TOP1. a 731-740 and may interact with
TAKVFRTYNA

SEQ ID NO: 1039 - protein, 10 aa, Arabidopsis Thaliana
  Sequence located in Unknown at 82-91 and may interact with
SGGGSRRSSS

SEQ ID NO: 1040 - protein, 10 aa, Arabidopsis Thaliana
  Sequence located in RPS6. a 287-296 and may interact with
APPSGAPAAG

SEQ ID NO: 1041 - protein, 10 aa, Arabidopsis Thaliana
  Sequence located in Unknown at 81-90 and may interact with
RSGGGSRRSS

SEQ ID NO: 1042 - protein, 10 aa, Arabidopsis Thaliana
  Sequence located in RPS6. a 286-295 and may interact with
AAPPSGAPAA

SEQ ID NO: 1043 - protein, 10 aa, Arabidopsis Thaliana
  Sequence located in Unknown at 186-195 and may interact with
SPERRRRSPS

SEQ ID NO: 1044 - protein, 10 aa, Arabidopsis Thaliana
  Sequence located in SUOX. a 285-294 and may interact with
AGLTAPPGRR

SEQ ID NO: 1045 - protein, 10 aa, Arabidopsis Thaliana
  Sequence located in Unknown at 181-190 and may interact with
RRRDRSPERR

SEQ ID NO: 1046 - protein, 10 aa, Arabidopsis Thaliana
  Sequence located in Unknown at 8-17 and may interact with
ATFRASVSSA

SEQ ID NO: 1047 - protein, 10 aa, Arabidopsis Thaliana
  Sequence located in Unknown at 182-191 and may interact with
RRDRSPERRR

SEQ ID NO: 1048 - protein, 10 aa, Arabidopsis Thaliana
  Sequence located in Unknown at 7-16 and may interact with
SATFRASVSS

SEQ ID NO: 1049 - protein, 10 aa, Arabidopsis Thaliana
  Sequence located in Unknown at 183-192 and may interact with
RDRSPERRRR

SEQ ID NO: 1050 - protein, 10 aa, Arabidopsis Thaliana
  Sequence located in Unknown at 6-15 and may interact with
TSATFRASVS

SEQ ID NO: 1051 - protein, 10 aa, Arabidopsis Thaliana
  Sequence located in Unknown at 184-193 and may interact with
DRSPERRRRS

SEQ ID NO: 1052 - protein, 10 aa, Arabidopsis Thaliana
  Sequence located in Unknown at 5-14 and may interact with
GTSATFRASV

SEQ ID NO: 1053 - protein, 10 aa, Arabidopsis Thaliana
  Sequence located in S1FA. a 52-61 and may interact with
LAPKSQLKAT

SEQ ID NO: 1054 - protein, 10 aa, Arabidopsis Thaliana
  Sequence located in T5I7.9. at 335-344 and may interact with
KSGLTLEFSC

SEQ ID NO: 1055 - protein, 10 aa, Arabidopsis Thaliana
  Sequence located in Unknown at 6-15 and may interact with
EKTSSGREYK

SEQ ID NO: 1056 - protein, 10 aa, Arabidopsis Thaliana
  Sequence located in Unknown at 16-25 and may interact with
FLGAAAALVF

SEQ ID NO: 1057 - protein, 10 aa, Arabidopsis Thaliana
  Sequence located in Unknown at 89-98 and may interact with
GTALAGRCGR

SEQ ID NO: 1058 - protein, 10 aa, Arabidopsis Thaliana
  Sequence located in SUOX. a 21-30 and may interact with
AAAAARQRSA

SEQ ID NO: 1059 - protein, 10 aa, Arabidopsis Thaliana
  Sequence located in Unknown at 382-391 and may interact with
RFEVWPYLET

SEQ ID NO: 1060 - protein, 10 aa, Arabidopsis Thaliana
  Sequence located in Unknown at 382-391 and may interact with
RFEVWPYLET

SEQ ID NO: 1061 - protein, 10 aa, Arabidopsis Thaliana
  Sequence located in Unknown at 203-212 and may interact with
LNTLQHTLRK

SEQ ID NO: 1062 - protein, 10 aa, Arabidopsis Thaliana
  Sequence located in T29M21. 5. at 308-317 and may interact
EVSKLVSQTF

SEQ ID NO: 1063 - protein, 10 aa, Arabidopsis Thaliana
  Sequence located in Unknown at 5-14 and may interact with
KAASLLISST

SEQ ID NO: 1064 - protein, 10 aa, Arabidopsis Thaliana
  Sequence located in ASA2 OR T27A16.21. at 342-351 and may
SATNQQTGRL

SEQ ID NO: 1065 - protein, 10 aa, Arabidopsis Thaliana
  Sequence located in Unknown at 833-842 and may interact with
ALKSRVDAAI

SEQ ID NO: 1066 - protein, 10 aa, Arabidopsis Thaliana
  Sequence located in ASA2 OR T27A16.21. at 250-259 and may
NGGVYTALKS

SEQ ID NO: 1067 - protein, 10 aa, Arabidopsis Thaliana
  Sequence located in Unknown at 214-223 and may interact with
RRSLDIERPT

SEQ ID NO: 1068 - protein, 10 aa, Arabidopsis Thaliana
  Sequence located in Unknown at 214-223 and may interact with
RRSLDIERPT

SEQ ID NO: 1069 - protein, 10 aa, Arabidopsis Thaliana
  Sequence located in Unknown at 214-223 and may interact with
RRSLDIERPT

SEQ ID NO: 1070 - protein, 10 aa, Arabidopsis Thaliana
  Sequence located in (TUBA3 R TUA3) AND (TUBA5 OR
RRSLDIERPT

SEQ ID NO: 1071 - protein, 10 aa, Arabidopsis Thaliana
  Sequence located in Unknown at 214-223 and may interact with
RRSLDIERPT

SEQ ID NO: 1072 - protein, 10 aa, Arabidopsis Thaliana
  Sequence located in TUBA6 O TUA6. at 214-223 and may
RRSLNIERPT

SEQ ID NO: 1073 - protein, 10 aa, Arabidopsis Thaliana
  Sequence located in (TUBA3 R TUA3) AND (TUBA5 OR
RRSLDIERPT

SEQ ID NO: 1074 - protein, 10 aa, Arabidopsis Thaliana
  Sequence located in (TUBA3 R TUA3) AND (TUBA5 OR
RRSLDIERPT

SEQ ID NO: 1075 - protein, 10 aa, Arabidopsis Thaliana
  Sequence located in (TUBA3 R TUA3) AND (TUBA5 OR
RRSLDIERPT

SEQ ID NO: 1076 - protein, 10 aa, Arabidopsis Thaliana
  Sequence located in TUBA6 O TUA6. at 214-223 and may
RRSLNIERPT

SEQ ID NO: 1077 - protein, 10 aa, Arabidopsis Thaliana
  Sequence located in TUBA6 O TUA6. at 214-223 and may
RRSLNIERPT

SEQ ID NO: 1078 - protein, 10 aa, Arabidopsis Thaliana
  Sequence located in TUBA6 O TUA6. at 214-223 and may
RRSLNIERPT

SEQ ID NO: 1079 - protein, 10 aa, Arabidopsis Thaliana
  Sequence located in CYP73A5 OR CYP73 OR T06B20.16. at 34-
PPGPIPIPIF

SEQ ID NO: 1080 - protein, 10 aa, Arabidopsis Thaliana
  Sequence located in VPS41. t 308-317 and may interact with
EDGDGDRPGR

SEQ ID NO: 1081 - protein, 10 aa, Arabidopsis Thaliana
  Sequence located in CYP73A5 OR CYP73 OR T06B20.16. at
IASSKPTGSE

SEQ ID NO: 1082 - protein, 10 aa, Arabidopsis Thaliana
  Sequence located in T8F5.20 at 4-13 and may interact with
DRTGLGSTRL

SEQ ID NO: 1083 - protein, 10 aa, Arabidopsis Thaliana
  Sequence located in CCT1. a 422-431 and may interact with
LSVYLEHLAT

SEQ ID NO: 1084 - protein, 10 aa, Arabidopsis Thaliana
  Sequence located in (AVA-P1 OR T4L20.300) AND AVA-P2
RGQVLEVDGE

SEQ ID NO: 1085 - protein, 10 aa, Arabidopsis Thaliana
  Sequence located in CCT1. a 423-432 and may interact with
SVYLEHLATT

SEQ ID NO: 1086 - protein, 10 aa, Arabidopsis Thaliana
  Sequence located in (AVA-P1 OR T4L20.300) AND AVA-P2
RRGQVLEVDG

SEQ ID NO: 1087 - protein, 10 aa, Arabidopsis Thaliana
  Sequence located in Unknown at 316-325 and may interact with
HCGGNGTRIC

SEQ ID NO: 1088 - protein, 10 aa, Arabidopsis Thaliana
  Sequence located in TOP1. a 57-66 and may interact with
MRSPVTSPNG

SEQ ID NO: 1089 - protein, 10 aa, Arabidopsis Thaliana
  Sequence located in TMKL1. t 297-306 and may interact with
SPGAVAGLVI

SEQ ID NO: 1090 - protein, 10 aa, Arabidopsis Thaliana
  Sequence located in T20K9.8 at 29-38 and may interact with
ARARDCPKDD

SEQ ID NO: 1091 - protein, 10 aa, Arabidopsis Thaliana
  Sequence located in A_TM021 04.13. at 341-350 and may
ESRLKLEARL

SEQ ID NO: 1092 - protein, 10 aa, Arabidopsis Thaliana
  Sequence located in A_TM021 04.13. at 341-350 and may
ESRLKLEARL

SEQ ID NO: 1093 - protein, 10 aa, Arabidopsis Thaliana
  Sequence located in F7J7.15 . at 263-272 and may interact with
TLSGKISPGT

SEQ ID NO: 1094 - protein, 10 aa, Arabidopsis Thaliana
  Sequence located in F4P9.44 OR T01B08.26. at 124-133 and may
SPRRNLATKG

SEQ ID NO: 1095 - protein, 10 aa, Arabidopsis Thaliana
  Sequence located in ADH2 OR ADHIII. at 238-247 and may
FGVNEFVNPK

SEQ ID NO: 1096 - protein, 10 aa, Arabidopsis Thaliana
  Sequence located in ADH2 OR ADHIII. at 238-247 and may
FGVNEFVNPK

SEQ ID NO: 1097 - protein, 10 aa, Arabidopsis Thaliana
  Sequence located in CAS1. a 93-102 and may interact with
VETTLKRGLD

SEQ ID NO: 1098 - protein, 10 aa, Arabidopsis Thaliana
  Sequence located in CAS1. a 93-102 and may interact with
VETTLKRGLD

SEQ ID NO: 1099 - protein, 10 aa, Arabidopsis Thaliana
  Sequence located in CNX3. a 111-120 and may interact with
SDMSKLTHVG

SEQ ID NO: 1100 - protein, 10 aa, Arabidopsis Thaliana
  Sequence located in CNX3. a 111-120 and may interact with
SDMSKLTHVG

SEQ ID NO: 1101 - protein, 10 aa, Arabidopsis Thaliana
  Sequence located in CYP84A1 OR FAH1 OR F23E13.110. at 45-
RTRRRSTAGP

SEQ ID NO: 1102 - protein, 10 aa, Arabidopsis Thaliana
  Sequence located in CYP84A1 OR FAH1 OR F23E13.110. at 45-
RTRRRSTAGP

SEQ ID NO: 1103 - protein, 10 aa, Arabidopsis Thaliana
  Sequence located in CTR1. a 261-270 and may interact with
RAVDSGVDSS

SEQ ID NO: 1104 - protein, 10 aa, Arabidopsis Thaliana
  Sequence located in CTR1. a 261-270 and may interact with
RAVDSGVDSS

SEQ ID NO: 1105 - protein, 10 aa, Arabidopsis Thaliana
  Sequence located in COB OR YTB. at 123-132 and may interact
TIGAGTGSNG

SEQ ID NO: 1106 - protein, 10 aa, Arabidopsis Thaliana
  Sequence located in COB OR YTB. at 123-132 and may interact
TIGAGTGSNG

SEQ ID NO: 1107 - protein, 10 aa, Arabidopsis Thaliana
  Sequence located in GAD1 OR GDH1 OR GAD. at 571-580 and
REILIDENLS

SEQ ID NO: 1108 - protein, 10 aa, Arabidopsis Thaliana
  Sequence located in GAD1 OR GDH1 OR GAD. at 571-580 and
REILIDENLS

SEQ ID NO: 1109 - protein, 10 aa, Arabidopsis Thaliana
  Sequence located in GAD1 OR GDH1 OR GAD. at 572-581 and
EILIDENLSQ

SEQ ID NO: 1110 - protein, 10 aa, Arabidopsis Thaliana
  Sequence located in GAD1 OR GDH1 OR GAD. at 570-579 and
LREILIDENL

SEQ ID NO: 1111 - protein, 10 aa, Arabidopsis Thaliana
  Sequence located in ERD11 O GST1. at 124-133 and may
SSDTAGGVGG

SEQ ID NO: 1112 - protein, 10 aa, Arabidopsis Thaliana
  Sequence located in ERD11 O GST1. at 124-133 and may
SSDTAGGVGG

SEQ ID NO: 1113 - protein, 10 aa, Arabidopsis Thaliana
  Sequence located in HMG1. a 576-585 and may interact with
RSSRDISGAT

SEQ ID NO: 1114 - protein, 10 aa, Arabidopsis Thaliana
  Sequence located in HMG1. a 576-585 and may interact with
RSSRDISGAT

SEQ ID NO: 1115 - protein, 10 aa, Arabidopsis Thaliana
  Sequence located in ORC2 OR F13M22.6. at 37-46 and may
AAASPGGGRR

SEQ ID NO: 1116 - protein, 10 aa, Arabidopsis Thaliana
  Sequence located in ORC2 OR F13M22.6. at 37-46 and may
AAASPGGGRR

SEQ ID NO: 1117 - protein, 10 aa, Arabidopsis Thaliana
  Sequence located in PAL1. a 674-683 and may interact with
REELGTELLT

SEQ ID NO: 1118 - protein, 10 aa, Arabidopsis Thaliana
  Sequence located in PAL1. a 674-683 and may interact with
REELGTELLT

SEQ ID NO: 1119 - protein, 10 aa, Arabidopsis Thaliana
  Sequence located in PUR2. a 302-311 and may interact with
EGEEASFFAL

SEQ ID NO: 1120 - protein, 10 aa, Arabidopsis Thaliana
  Sequence located in PUR2. a 302-311 and may interact with
EGEEASFFAL

SEQ ID NO: 1121 - protein, 10 aa, Arabidopsis Thaliana
  Sequence located in Unknown at 97-106 and may interact with
GTASTLTRGE

SEQ ID NO: 1122 - protein, 10 aa, Arabidopsis Thaliana
  Sequence located in Unknown at 123-132 and may interact with
ARRGSQRTPL

SEQ ID NO: 1123 - protein, 10 aa, Arabidopsis Thaliana
  Sequence located in RLK5 OR F21O9.180. at 26-35 and may
ASVPSGGDGG

SEQ ID NO: 1124 - protein, 10 aa, Arabidopsis Thaliana
  Sequence located in RLK5 OR F21O9.180. at 26-35 and may
ASVPSGGDGG

SEQ ID NO: 1125 - protein, 10 aa, Arabidopsis Thaliana
  Sequence located in RLK5 OR F21O9.180. at 39-48 and may
ASATSGGGGG

SEQ ID NO: 1126 - protein, 10 aa, Arabidopsis Thaliana
  Sequence located in RLK5 OR F21O9.180. at 39-48 and may
ASATSGGGGG

SEQ ID NO: 1127 - protein, 10 aa, Arabidopsis Thaliana
  Sequence located in RBP31 O RNPT. at 59-68 and may interact
VSLSLKTKTH

SEQ ID NO: 1128 - protein, 10 aa, Arabidopsis Thaliana
  Sequence located in RBP31 O RNPT. at 59-68 and may interact
VSLSLKTKTH

SEQ ID NO: 1129 - protein, 10 aa, Arabidopsis Thaliana
  Sequence located in RPB205 R RPII OR RPB1. at 617-626 and
IPKQINLLRY

SEQ ID NO: 1130 - protein, 10 aa, Arabidopsis Thaliana
  Sequence located in RPB205 R RPII OR RPB1. at 617-626 and
IPKQINLLRY

SEQ ID NO: 1131 - protein, 10 aa, Arabidopsis Thaliana
  Sequence located in RPB205 R RPII OR RPB1. at 591-600 and
IPKQINLLRY

SEQ ID NO: 1132 - protein, 10 aa, Arabidopsis Thaliana
  Sequence located in RPB205 R RPII OR RPB1. at 591-600 and
IPKQINLLRY

SEQ ID NO: 1133 - protein, 10 aa, Arabidopsis Thaliana
  Sequence located in RPOPT. t 378-387 and may interact with
FRHRFKTVTK

SEQ ID NO: 1134 - protein, 10 aa, Arabidopsis Thaliana
  Sequence located in RPOPT. t 378-387 and may interact with
FRHRFKTVTK

SEQ ID NO: 1135 - protein, 10 aa, Arabidopsis Thaliana
  Sequence located in Unknown at 382-391 and may interact with
RFEVWPYLET

SEQ ID NO: 1136 - protein, 10 aa, Arabidopsis Thaliana
  Sequence located in Unknown at 382-391 and may interact with
RFEVWPYLET

SEQ ID NO: 1137 - protein, 10 aa, Arabidopsis Thaliana
  Sequence located in Unknown at 214-223 and may interact with
RRSLDIERPT

SEQ ID NO: 1138 - protein, 10 aa, Arabidopsis Thaliana
  Sequence located in Unknown at 214-223 and may interact with
RRSLDIERPT

SEQ ID NO: 1139 - protein, 10 aa, Arabidopsis Thaliana
  Sequence located in (TUBA3 R TUA3) AND (TUBA5 OR
RRSLDIERPT

SEQ ID NO: 1140 - protein, 10 aa, Arabidopsis Thaliana
  Sequence located in (TUBA3 R TUA3) AND (TUBA5 OR
RRSLDIERPT

SEQ ID NO: 1141 - protein, 10 aa, Arabidopsis Thaliana
  Sequence located in TUBA6 O TUA6. at 214-223 and may
RRSLNIERPT

SEQ ID NO: 1142 - protein, 10 aa, Arabidopsis Thaliana
  Sequence located in TUBA6 O TUA6. at 214-223 and may
RRSLNIERPT

SEQ ID NO: 1143 - protein, 10 aa, Arabidopsis Thaliana
  Sequence located in A_TM021 04.13. at 341-350 and may
ESRLKLEARL

SEQ ID NO: 1144 - protein, 10 aa, Arabidopsis Thaliana
  Sequence located in A_TM021 04.13. at 341-350 and may
ESRLKLEARL

(19) United States
(12) Patent Application Publication
(10) Pub. No.: US 2003/0148368 A1
(43) Pub. Date: Aug. 7, 2003

(54) Complementary peptide ligands generated from plant genomes

(76) Inventors: Gareth W. Roberts, Cambridge (GB); Jonathan R. Heal, Highbury (GB)

Correspondence Address: FINNEGAN, HENDERSON, FARABOW, GARRETT &, DUNNER LLP, 1300 I STREET, NW, WASHINGTON, DC, 20006, US

(21) Appl. No.: 09/572,270

(22) Filed: May 17, 2000

(30) Foreign Application Priority Data
Dec. 13, 1999 (GB) 9929469.6

(51) Int. Cl. G01N 33/53; G06F 19/00; G01N 33/48; G01N 33/50; C07K 14/415

(52) U.S. Cl. 435/7.1; 530/370; 702/19

(57) ABSTRACT

In the current invention the application of our novel informatics approach to the databases containing nucleotide and peptide sequences from plant genomes generates the sequence of many peptides which form the basis of an innovative and novel approach to developing new therapeutic agents.

This invention claims the use of specific complementary peptides to the proteins encoded in the genomes of plants as reagents for agricultural discovery programmes.

The analysis of plant genomes has become a key area of research within the last few years. It is predicted that the model organism Arabidopsis thaliana will be completely sequenced by 2001 (Arabidopsis Genome Initiative). The application of novel approaches that utilise genomic data will aid in the generation of more efficient crop protection and in the improvement of desired traits in seeds.

A process for the searching and analysis of protein and nucleotide sequence databases has been identified. This invention describes the application of this process to the databases containing nucleotide and protein sequence data from plant genomes.

This invention claims the use of specific complementary peptides to the proteins encoded in plant genomes as tools for agriculrural research and development.

### BACKGROUND

Specific protein interactions are critical events in most biological processes and a clear idea of the way proteins interact, their three dimensional structure and the types of molecules which might block or enhance interaction are critical aspects of the science of drug discovery in the pharmaceutical industry.

Proteins are made up of strings of amino acids and each amino acid in a string is coded for by a triplet of nucleotides present in DNA sequences (Stryer 1997). The linear sequence of DNA code is read and translated by a cell's synthetic machinery to produce a linear sequence of amino acids that then fold to form a complex three-dimensional protein.

In general it is held that the primary structure of a protein determines its tertiary structure. A large volume of work supports this view and many sources of software are available to the scientists in order to produce models of protein structures (Sansom 1998). In addition, a considerable effort is underway in order to build on this principle and generate a definitive database demonstrating the relationships between primary and tertiary protein structures. This endeavour is likened to the human genome project and is estimated to have a similar cost (Gaasterland 1998).

The binding of large proteinaceous signalling molecules (such as hormones) to cellular receptors regulates a substantial portion of the control of cellular processes and functions. These protein-protein interactions are distinct from the interaction of substrates to enzymes or small molecule ligands to seven-transmembrane receptors. Protein-protein interactions occur over relatively large surface areas, as opposed to the interactions of small molecule ligands with serpentine receptors, or enzymes with their substrates, which usually occur in focused “pockets” or “clefts”. Thus, protein-protein targets are non-traditional and the pharmaceutical community has had very limited success in developing drugs that bind to them using currently available approaches to lead discovery. High throughput screening technologies in which large (combinatorial) libraries of synthetic compounds are screened against a target protein(s) have failed to produce a significant number of lead compounds.

Diseases caused by bacteria and fungi are currently some of the major factors limiting crop production worldwide. Consequently, much research is focussed on those genes that can confer significant levels of disease resistance. Much of this work is focussed on understanding naturally-occuring plant signalling pathways that control pathogen resistance, and on identifying genes encoding antifungal proteins. Targetting protein-protein interactions or mimicing protein signalling molecules may alter expression of antifungal proteins and assist in combatting diseases in plants.

It is generally believed that because the binding interfaces between proteins are very large, traditional approaches to drug screening or design have not been successful. In fact, for most protein-protein interactions, only small subsets of the overall intermolecular surfaces are important in defining binding affinity.

“One strongly suspects that the many crevices, canyons, depressions and gaps, that punctuate any protein surface are places that interact with numerous micro- and macro-molecular ligands inside the cell or in the extra-cellular spaces, the identity of which is not known” (Goldstein 1998).

Despite these complexities, recent evidence suggests that protein-protein interfaces are tractable targets for drug design when coupled with suitable functional analysis and more robust molecular diversity methods. For example, the interface between hGH and its receptor buries˜1300 Sq. Angstroms of surface area and involves 30 contact side chains across the interface. However, alanine-scanning mutagenesis shows that only eight side-chains at the centre of the interface (covering an area of about 350 Sq. Angstroms) are crucial for affinity. Such “hot spots” have been found in numerous other protein-protein complexes by alanine-scanning, and their existence is likely to be a general phenomenon.

The problem is therefore to define the small subset of regions that define the binding or functionality of the protein.

The important commercial reason for this is that a more efficient way of doing this would greatly accelerate the process of developing crops with improved characteristics such as nutritional content, disease resistance etc.

These complexities are not insoluble problems and newer theoretical methods should not be ignored in the drug design process. Nonetheless, in the near future there are no good algorithms that allow one to predict protein binding affinities quickly, reliably, and with high precision (Sunesis website www.sunesis.com Aug. 17, 1999).

A process for the analysis of whole genome databases has been developed. Significant utility can be achieved within the crop protection and seeds industry by searching and analysing protein and nucleotide sequence databases to identify complementary peptides which interact with their relevant target proteins.

These novel peptides can be used as lead ligands to facilitate the development of novel herbicides and pesticides and improve crop yield and disease resistance. This invention describes the application of this process to the databases containing nucleotide and protein sequence data from plant genomes.

The process has been described in patent application No. GB 9927485.4, filed Nov. 19, 1999 for use in analysing, and manipulating the sequence data (both DNA and protein) found in large databases and its utility in conducting systematic searches to identify the sequences which code for the key intermolecular surfaces or “hot spots” on specific protein targets.

### THE INVENTION

In the current invention the application of our novel informatics approach to the databases containing nucleotide and peptide sequences from plant genomes generates the sequence of many peptides which form the basis of an innovative and novel approach to developing new therapeutic agents.

This invention claims the use of specific complementary peptides to the proteins encoded in the genomes of plants as reagents for agricultural discovery programmes.

### APPLICATION OF THE DATA MINING PROCESS TO THE ANALYSIS OF PLANT GENOMES

We have applied our computational approach with its novel algorithms for generating complementary peptides, patent application number GB 9927485.4 to the known plant nucleotide and peptide sequence databases.

Whole genome sequences represent a huge resource of data for the discovery and utilisation of biologically important complementary peptides. For example, there are at least 45,000 entries for green plants in Genbank and the Arabidopsis Genome Initiative (AGI) has recently been launched to complete sequencing of the 130 Mb Arabidopsis genome by the year 2001. Plant nucleotide and protein sequence data is publicly available in a number of large databases (EXAMPLE 1), and these are continually updated as more sequence becomes available.

The catalogues detailed in this patent cover all available plant genomes. A table detailing a sample of the plant genome databases that have been processed using our method is shown in EXAMPLE 2. Many of these species are of major economic significance and identification of the genes and pathways that control traits of agricultural and biological interest is an important goal.

Identification of complementary peptides within and between proteins in plant species will aid in the identification of agronomically important traits such as disease resistance, resistance to adverse climates and increased yields. In addition, knowledge of interacting proteins will allow prediction of the potential toxic effects of, for example, genetically modified plants in humans and other species.

The utility of this approach is outlined in EXAMPLE 3.

A catalogue of complementary inter molecular peptides (average 3 per gene) was generated for each gene within a genome (see EXAMPLE 4).

Sets of shorter ‘daughter’ sequences of frame size 5, 6, 7, 8 or 9 can also be derived from these sequences (EXAMPLE 5). A further set of intra-molecular complementary peptide sequences was also generated for each gene within a genome (see EXAMPLE 6).

Sets of shorter ‘daughter’ sequences of frame size 5, 6, 7, 8 or 9 can also be derived from these sequences (EXAMPLE 7).

Each complementary peptide sequence has a unique identifying number in the catalog and peptides are categorised as either intra-molecular or inter-molecular peptides within each genome as shown in the table below (and in EXAMPLES 4, 6 and in the genomes noted in EXAMPLES 8 and 9):

| | Intra molecular | Inter molecular |
|---|---|---|
| Genome | peptides | peptides |
| Arabidopsis thaliana | 1095-1143 | 1-1094 |
| Oilseed rape (Brassica napus) | | |
| Alfalfa (Medicago sativa) | | |
| Rice (Oryza sativa) | | |
| Sorghum (Sorghum bicolor) | | |
| Maize (Zea mays) | | |
| Loblolly Pine (Pinus taeda L.) | | |
| Barley (Hordeum vulgare) | | |
| Pearl Millet (Pennisetum | | |
| glaucum) | | |
| Finger millet (Eleusine | | |
| coracana) | | |
| Foxtail millet (Setaria italica) | | |
| Forage grasses, (Lolium | | |
| Perenne, Lolium multiflorum and | | |
| Festuca Pratensis) | | |
| Lotus japonicus (a model | | |
| legume) | | |
| Barrel medic (Medicago | | |
| truncatula) | | |
| Pea (Pisum sativum) | | |
| Cotton (Gossypium hirsutum) | | |
| Soybean (Glycine max) | | |
| Wheat (Triticum aestivum) | | |

Utilizing our novel approach we were able to establish the sequences of complementary peptides that have the potential to interact with and alter the functionality of the relevant protein coded for by its gene. Furthermore the second analysis provides information as to the regions on other proteins which might interact with the first protein (its ‘molecular partners’ in physiological functions).

The peptide sequences described herein can be readily made into peptides by a multitude of methods. The peptides made from the sequences described in this patent will have considerable utility as tools for functional genomics studies, reagents for the configuration of high-throughput screens, a starting point for further chemical manipulation, peptide mimetics, and active agents in their own right.

The process of patent application No. GB9927485.4 will now be described below. The examples of this present application are the result of applying that process to a selected plant genome database (Arabidopsis thaliana): it will be readily appreciated that use of the process on other databases will yield peptide sequences and catalogues of intra- and inter-molecular complementary peptides specific to the other plant databases (e.g. the databases listed in EXAMPLES 1 and 2).

The current problems associated with design of complementary peptides are:

A lack of understanding of the forces of recognition between complementary peptides

An absence of software tools to facilitate searching and selecting complementary peptide pairs from within a protein database

A lack of understanding of statistical relevance/distribution of naturally encoded complementary peptides and how this corresponds to functional relevance.

Based on these shortfalls, our process provides the following technological advances in this field:

A mini library approach to define forces of recognition between human Interleukin (IL) 1 and its complementary peptides.

A high throughput computer system to analyse an entire database for intra/inter-molecular complementary regions.

Studies into preferred complementary peptide pairings between IL-1 and its complementary ligand reveal the importance of both the genetic code and complementary hydropathy for recognition. Specifically, for our example, the genetic code for a region of protein codes for the complementary peptide with the highest affinity. An important observation is that this complementary peptide maps spatially and by residue hydropathic character to the interacting portion of the IL-1R receptor, as elucidated by the X-ray crystal structure Brookhaven reference pdb2itb.ent.

Using these novel observations as guiding principles for analysis, we have developed a computational analysis system to evaluate the statistical and functional relevance of intra/inter-molecular complementary sequences.

This process provides significant benefits for those interested in:

The analysis and acquisition of peptide sequences to be used in the understanding of protein-protein interactions.

The development of peptides or small molecules which could be used to manipulate these interactions.

The advantages of this process to previous work in this field include:

Using a valid statistical model. Previously, complementary mappings within protein structures has been statistically validated by assuming that the occurrence of individual amino acids is equally weighted at 1/20 (Baranyi, 1995). Our statistical model takes into account the natural occurrence of amino acids and thus generates probabilities dependent on sequence rather than content per se.

Facilitation of batch searching of an entire database. Previously, investigations into the significance of naturally encoded complementary related sequences have been limited to small sample sizes with non-automated methods. The invention allows for analysis of an entire database at a time, overcoming the sampling problem, and providing for the first time an overview or ‘map’ of complementary peptide sequences within known protein sequences.

The ability to map complementary sequences as a function of frame size and percentage antisense amino acid content. Previously, no consideration has been given to the significance of the frame length of complementary sequences. Our process produces a statistical map as a function of frame size and percentage complementary residue content such that the statistical importance of how nature selects these frames may be evaluated.

### BRIEF DESCRIPTION OF DRAWINGS

The process is described with reference to accompanying drawings. In the drawings, like reference numbers indicate identical or functionally similar elements.

FIG. (1) shows a block diagram illustrating one embodiment of a method of the present invention

FIG. (2) shows a block diagram illustrating one embodiment for carrying out Step 4 in FIG. (1)

FIG. (3) shows a block diagram illustrating one embodiment for carrying out Step 5 in FIG (1)

FIG. (4) shows a block diagram illustrating one embodiment for carrying out Step 8 in FIGS. (2) and (3)

FIG. (5) shows a block diagram illustrating one embodiment for carrying out Step 8 in FIGS. (2) and (3)

FIG. (6) shows a block diagram illustrating one embodiment for carrying out Step 6 in FIG. (1)

### A DESCRIPTION OF THE ANALYTICAL PROCESS

The software, ALS (antisense ligand searcher), performs the following tasks:

Given the input of two amino acid sequences, calculates the position, number and probability of the existence of intra- (within a protein) and inter- (between proteins) molecular antisense regions. ‘Antisense’ refers to relationships between amino acids specified in EXAMPLES 10 and 11 (both 5′→3′ derived and 3′→5′ derived coding schemes).

Allows sequences to be inputted manually through a suitable user interface (UI) and also through a connection to a database such that automated, or batch, processing can be facilitated.

Provides a suitable database to store results and an appropriate interface to allow manipulation of this data.

Allows generation of random sequences to function as experimental controls.

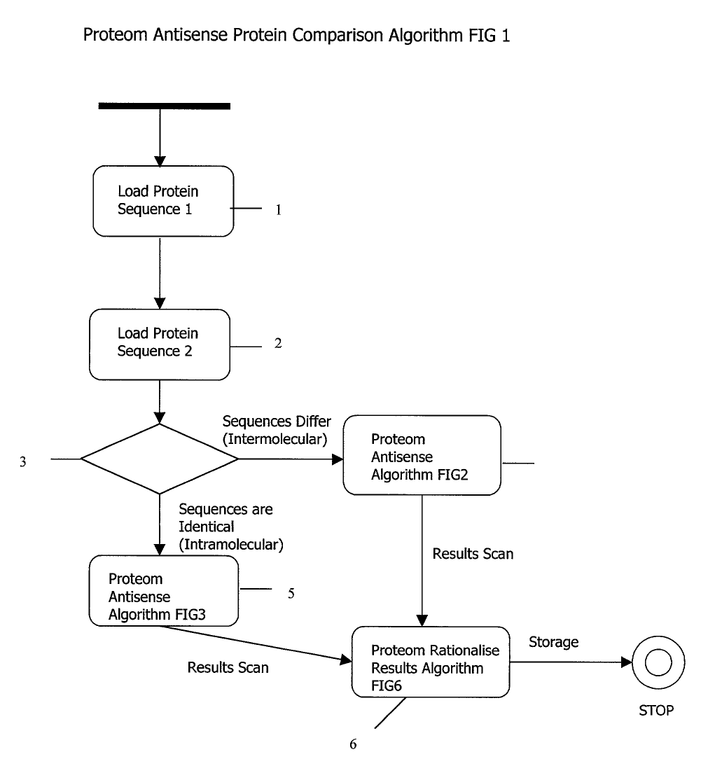

Diagrams describing the algorithms involved in this software are shown in FIGS. 1-5.

### DETAILED DESCRIPTION

1. Overview

The present process is directed toward a computer-based process, a computer-based system and/or a computer program product for analysing antisense relationships between protein or DNA sequences. The method of the embodiment provides a tool for the analysis of protein or DNA sequences for antisense relationships. This embodiment covers analysis of DNA or protein sequences for intramolecular (within the same sequence) antisense relationships or inter-molecular (between 2 different sequences) antisense relationships. This principle applies whether the sequence contains amino acid information (protein) or DNA information, since the former may be derived from the latter.

The overall process is to facilitate the batch analysis of an entire genome (collection of genes/and or protein sequences) for every possible antisense relationship of both inter- and intra-molecular nature. For the purpose of example it will be described here how a protein sequence database may be analysed by the methods described.

The program runs in two modes. The first mode (Intermolecular) is to select the first protein sequence in the databases and then analyse the antisense relationships between this sequence and all other protein sequences, one at a time. The program then selects the second sequence and repeats this process. This continues until all of the possible relationships have been analysed. The second mode (Intramolecular) is where each protein sequence is analysed for antisense relationships within the same protein and thus each sequence is loaded from the database and analysed in turn for these properties. Both operational modes use the same core algorithms for their processes. The core algorithms are described in detail below.

An example of the output from this process is a list of proteins in the database that contain highly improbable numbers of intramolecular antisense frames of size 10 (frame size is a section of the main sequence, it is described in more detail below).

2. Method

For the purpose of example protein sequence 1 is ATRGRDSRDERSDERTD and protein sequence 2 is GTFRTSREDSTYSGDTDFDE (universal 1 letter amino acid codes used).

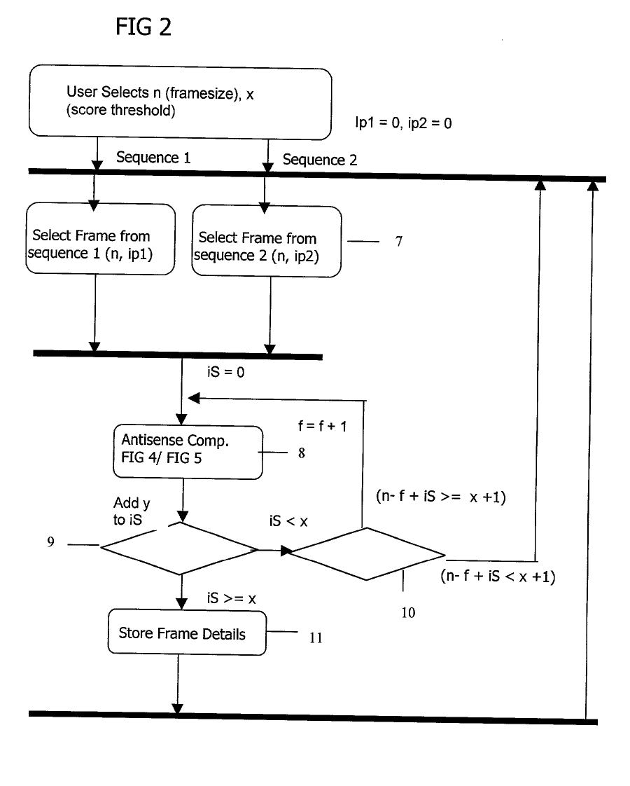

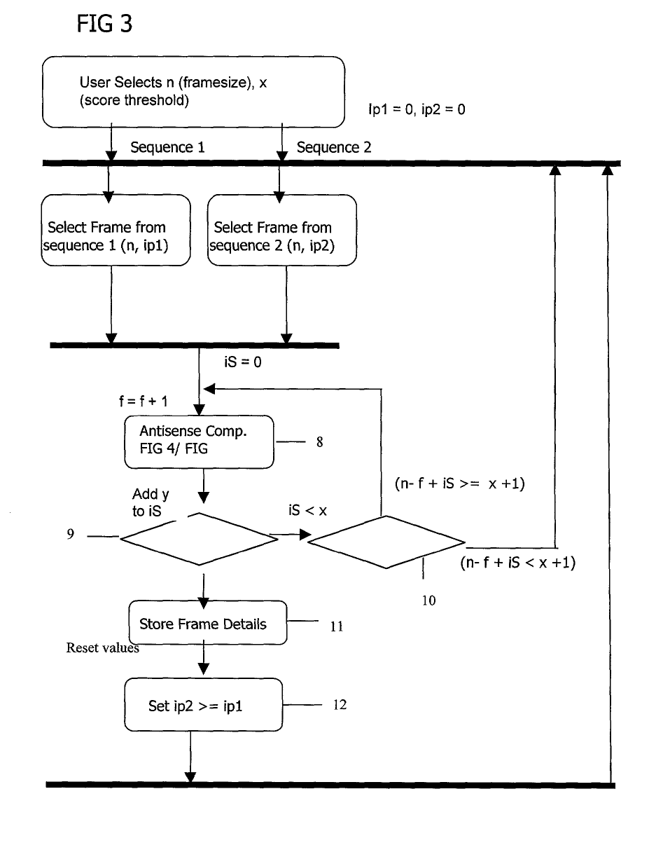

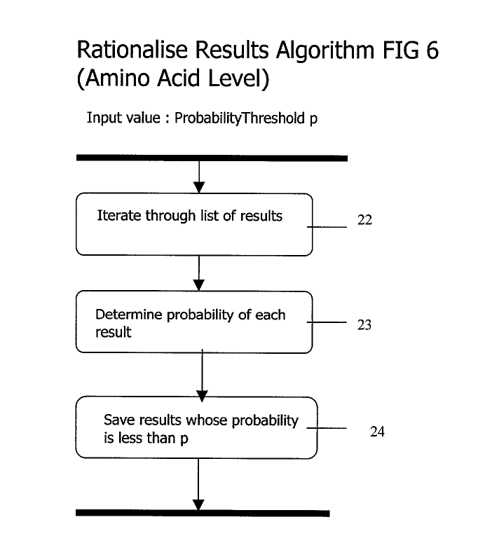

In step 1 (see FIG. 1), a protein sequence, sequence 1, is loaded. The protein sequence consists of an array of universally recognised amino acid one letter codes, e.g. ‘ADTRGSRD’. The source of this sequence can be a database, or any other file type. Step 2, is the same operation as for step 1, except sequence 2 is loaded. Decision step 3 involves comparing the two sequences and determining whether they are identical, or whether they differ. If they differ, processing continues to step 4, described in FIG. 2, otherwise processing continues to step 5, described in FIG. 3. Step 6 analyses the data resulting from either step 4, or step 5, and involves an algorithm described in FIG. 6.

Description of parameters used in FIG. 2

| Name | Description |
|---|---|
| N | Framesize - the number of amino acids that make up each ‘frame’ |
| X | Score threshold - the number of amino acids that have to fulfil the |
| | antisense criteria within a given frame for that frame to be stored |
| | for analysis |
| Y | Score of individual antisense comparison (either 1 or 0) |
| IS | Running score for frame - (sum of y for frame) |
| ip1 | Position marker for Sequence 1 - used to track location of selected |
| | frame for sequence 1 |
| ip2 | Position marker for Sequence 2 - used to track location of selected |
| | frame for sequence 1 |
| F | Current position in frame |

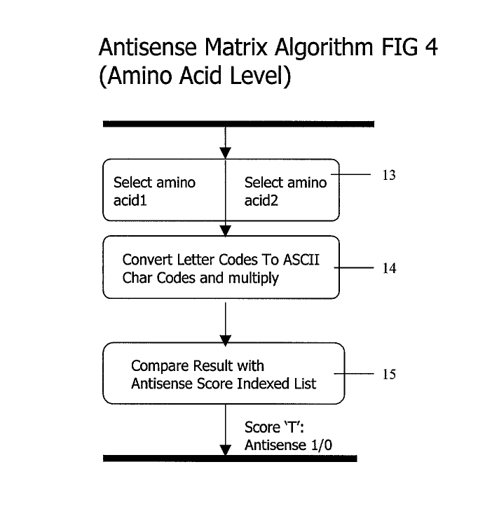

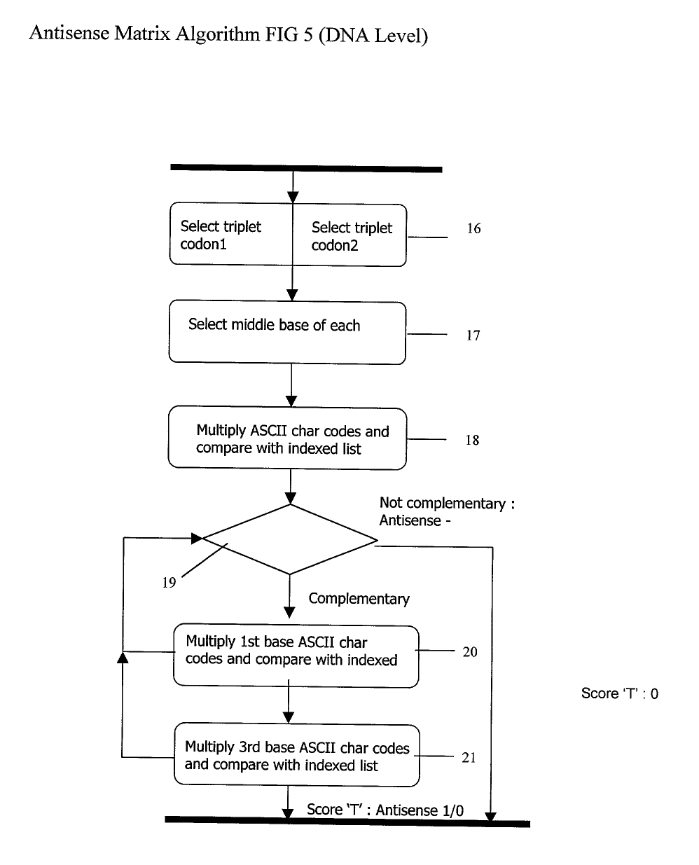

In Step 7, a ‘frame’ is selected for each of the proteins selected in steps 1 and 2. A ‘frame’ is a specific section of a protein sequence. For example, for sequence 1, the first frame of length ‘5′’ would correspond to the characters ‘ATRGR’. The user of the program decides the frame length as an input value. This value corresponds to parameter ‘n’ in FIG. 2. A frame is selected from each of the protein sequences (sequence 1 and sequence 2). Each pair of frames that are selected are aligned and frame position parameter f is set to zero. The first pair of amino acids are ‘compared’ using the algorithm shown in FIG. 4/FIG. 5. The score output from this algorithm (y, either one or zero) is added to a aggregate score for the frame iS. In decision step 9 it is determined whether the aggregate score iS is greater than the Score threshold value (x). If it is then the frame is stored for further analyisis. If it is not then decision step 10 is implemented. In decision step 10, it is determined whether it is possible for the frame to yield the score threshold (x). If it can, the frame processing continues and f is incremented such that the next pair of amino acids are compared. If it cannot, the loop exits and the next frame is selected. The position that the frame is selected from the protein sequences is determined by the parameter ip1 for sequence 1 and ip2 for sequence 2 (refer to FIG. 2). Each time steps 7 to 10 or 7 to 11 are completed, the value of ip1 is zeroed and then incremented until all frames of sequence 1 have been analysed against the chosen frame of sequence 2. When this is done, ip2 is then incremented and the value of ip1 is incremented until all frames of sequence 1 have been analysed against the chosen frame of sequence 2. This process repeats and terminates when ip2 is equal to the length of sequence 2. Once this process is complete, sequence 1 is reversed programmatically and the same analysis as described above is repeated. The overall effect of repeating steps 7 to 11 using each possible frame from both sequences is to facilitate step 8, the antisense scoring matrix for each possible combination of linear sequences at a given frame length.

FIG. 3 shows a block diagram of the algorithmic process that is carried out in the conditions described in FIG. 1. Step 12 is the only difference between the algorithms FIG. 2 and FIG. 3. In step 12, the value of ip2 (the position of the frame in sequence 2) is set to at least the value of ip1 at all times since as sequence 1 and sequence 2 are identical, if ip2 is less than ip1 then the same sequences are being searched twice.

FIGS. 4 and 5 describe the process in which a pair of amino acids (FIG. 4) or a pair of triplet codons are assessed for an antisense relationship. The antisense relationships are listed in EXAMPLES 9 and 10. In step 13, the currently selected amino acid from the current frame of sequence 1 and the currently selected amino acid from the current frame of sequence 2 (determined by parameter ‘f’ in FIGS. 2/3) are selected. For example, the first amino acid from the first frame of sequence 1 would be ‘A’ and the first amino acid from the first frame of sequence 2 would be ‘G’. In step 14, the ASCII character codes for the selected single uppercase characters are determined and multiplied and, in step 15, the product compared with a list of precalculated scores, which represent the antisense relationships in EXAMPLES 10 and 11. If the amino acids are deemed to fulfil the criteria for an antisense relationship (the product matches a value in the precalculated list) then an output parameter ‘T’ is set to 1, otherwise the output parameter is set to zero.

Steps 16-21 relate to the case where the input sequences are DNA/RNA code rather the protein sequence. For example sequence 1 could be AAATTTAGCATG and sequence 2 could be TTTAAAGCATGC. The domain of the current invention includes both of these types of information as input values, since the protein sequence can be decoded from the DNA sequence, in accordance with the genetic code. Steps 16-21 determine antisense relationships for a given triplet codon. In step 16, the currently selected triplet codon for both sequences is ‘read’. For example, for sequence 1 the first triplet codon of the first frame would be ‘AAA’, and for sequence 2 this would be ‘TTT’. In step 17, the second character of each of these strings is selected. In step 18, the ASCII codes are multiplied and compared, in decision step 19, to a list to find out if the bases selected are ‘complementary’, in accordance with the rules of the genetic code. If they are, the first bases are compared in step 20, and subsequently the third bases are compared in step 21. Step 18 then determines whether the bases are ‘complementary’ or not. If the comparison yields a ‘non-complementary’ value at any step the routine terminates and the output score ‘T’ is set to zero. Otherwise the triplet codons are complementary and the output score T=1.

FIG. 6 illustrates the process of rationalizing the results after the comparison of 2 protein or 2 DNA sequences. In step 22, the first ‘result’ is selected. A result consists of information on a pair of frames that were deemed ‘antisense’ in FIG's 2 or 3. This information includes location, length, score (i.e the sum of scores for a frame) and frame type (forward or reverse, depending on orientation of sequences with respect to one another). In step 23, the frame size, the score values and the length of the parent sequence are then used to calculate the probability of that frame existing. The statistics, which govern the probability of any frame existing, are described in the next section and refer to equations 1-4. If the probability is less than a user chosen value ‘p’, then the frame details are ‘stored’ for inclusion in the final result set (step 24).

### Statistical Basis of Program Operation

The number of complementary frames in a protein sequence can be predicted from appropriate use of statistical theory.

The probability of any one residue fitting the criteria for a complementary relationship with any other is defined by the groupings illustrated in EXAMPLE 10. Thus, depending on the residue in question, there are varying probabilities for the selection of a complementary amino acid. This is a result of an uneven distribution of possible partners. For example possible complementary partners for a tryptophan residue include only proline whilst glycine, serine, cysteine and arginine all fulfil the criteria as complementary partners for threonine. The probabilities for these residues aligning with a complementary match are thus 0.05 and 0.2 respectively. The first problem in fitting, an accurate equation to describe the expected number of complementary frames within any sequence is integrating these uneven probabilities into the model. One solution is to use an average value of the relative abundance of the different amino acids in natural sequences. This is calculated by equation 1

v=ΣR*N 1

Where v=probability sum, R=fractional abundance of amino acid in e.coli proteins, N=number of complementary partners specified by genetic code.

This value (p) is calculated as 2.98. The average probability (p) of selecting a complementary amino acid is thus 2.98/20=0.149.

For a single ‘frame’ of size (n) the probability (C) of pairing a number of complementary amino acids (r) can be described by the binomial distribution (equation 2)

$$C = \frac{n !}{( n - r ) ! r !} {{p}^{r} ( 1 - p )}^{n - r} \qquad 2$$

With this information we can predict that the expected number (Ex) of complementary frames in a protein to be:

$$Ex = 2 {( S - n )}^{2} \frac{n !}{( n - r ) ! r !} {{p}^{r} ( 1 - p )}^{n - r} \qquad 3$$

Where S=protein length, n=frame size, r=number of complementary residues required for a frame and p=0.149. If r=n, representing that all amino acids in a frame have to fulfil a complementary relationship, the above equation simplifies to:

Ex=2(S−n)²pⁿ 4

For a population of randomly assembled amino acid chains of a predetermined length we would expect the number of frames fulfilling the complementary criteria in the search algorithm to vary in accordance with a normal distribution.

Importantly, it is possible to standardise results such that given a calculated mean ( ) and standard deviation ( ) for a population it is possible to determine the probability of any specific result occurring. Standardisation of the distribution model is facilitated by the following relation:

$$Z = \frac{X - μ}{σ} \qquad 5$$

Where X is an single value (result) in a population.

If we are considering complementary frames with a single protein structure then the above statistical model requires further analysis. In particular, the possibility exists that a region may be complementary to itself, as indicated in the diagram below.

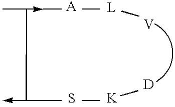

Reverse turn motifs within proteins. A region of protein may be complementary to itself. In this scenario, A-S, L-K and V-D are complementary partners. A six amino acid wide frame would thus be reported (in reverse orientation). A frame of this type is only specified by half of the residues in the frame. Such a frame is called a reverse turn.

In this scenario, once half of the frame length has been selected with complementary partners, there is a finite probability that those partners are the sequential neighbouring amino acids to those already selected. The probability of this occurring in any protein of any sequence is:

Ex=p^{f}^{^{/2}}(S−f) 7

Where f is the frame size for analysis, and S is the sequence length and p is the average probability of choosing an antisense amino acid.

The software of the embodiment incorporates all of the statistical models reported above such that it may assess whether a frame qualifies as a forward frame, reverse frame, or reverse turn.

### EXAMPLE 1

PROTEIN AND NUCLEOTIDE SEQUENCE DATABASES
AMENABLE FOR ANALYSIS USING THE PROCESS

| Database | Description | Web site address |
|---|---|---|
| Genbank at NCBI | The Genbank database is a repository for | Http://www.ncbi.nlm.nih |
| (National Center for | nucleotide data. | .gov/ |
| Biotechnology Information) | | |
| EMBL | The EMBL database is a repository for | http://www.ebi.ac.uk |
| | nucleotide data. | |
| DbEST | Database of ESTs (Expressed Sequence | Http://www.ncbi.nlm.nih |
| | Tag) from all species | .gov/dbEST/index.html |
| SWISS-PROT | Curated protein sequence database | http://www.expasy.ch/s |
| | | prot/sprot-top.html |
| TrEMBL | Supplement of SWISS-PROT that contains | http://www.expasy.ch/s |
| | all the translations of EMBL nucleotide | prot/sprot-top.html |
| | sequence entries not yet integrated in | |
| | SWISS-PROT. | |
| PMIC | Plant Molecular Informatics Centre | Http://www.cbc.med.um |
| | | n.edu/ResearchProjects |
| | | /seq.proc.html |

### EXAMPLE 2

CATALOG OF PLANT GENOMES

| Genome | Web link |
|---|---|
| Thale cress or mouse eared cress | http://genome-www.stanford.edu/Arabidopsis/ |
| (Arabidopsis thaliana) | http://genome.wustl.edu/gsc/Projects/thaliana.shtml |
| | http://www.cbc.umn.edu/ResearchProjects/Arabidopsis/index.html |
| | http://www.cbs.dtu.dk/databases/ARACLEAN/ |
| | AraClean is an corrected and redundancy reduced database of |
| | Arabidopsis thaliana sequences extracted from GenBank |
| Oilseed rape (Brassica napus) | http://synteny.nott.ac.uk/brassica.html |
| Alfalfa (Medicago sativa) | http://probe.nal.usda.gov:8000/plant/aboutalfagenes.html |
| Rice (Oryza sativa) | Rice Genome Project http://www.staff.or.jp/ and |
| | http://www.cbc.umn.edu/ResearchProjects/Rice/index.html |
| | http://www.tigr.org/tdb/ogi/ |
| Sorghum (Sorghum bicolor) | |
| Maize (Zea mays) | http://sequence-www.stanford.edu/group/maize/maize2.html |
| | and http://www.zmdb.iastate.edu/ |
| | and Maize Genome database at http://www.agron.missouri.edu/ |
| Loblolly Pine (Pinus taeda L.) | http://www.cbc.umn.edu/ResearchProjects/Pine/DOE pine/index.htm |
| | l |
| | http://dendrome.ucdavis.edu/ |
| Barley (Hordeum vulgare) | http://synteny.nott.ac.uk/barley.html |
| Pearl Millet (Pennisetum glaucum) | http://synteny.nott.ac.uk/millet.html |
| Finger millet (Eleusine coracana) | http://synteny.nott.ac.uk/millet.html |
| Foxtail millet (Setaria italica) | http://synteny.nott.ac.uk/millet.html |
| Forage grasses, (Lolium Perenne, Lolium | http://synteny.nott.ac.uk/grass.html |
| multiflorum and Festuca Pratensis) | |
| Lotus japonicus (a model legume) | http://www.jic.bbsrc.ac.uk/sainsbury-lab/martin- |
| | parniske/mphome.htm |
| Barrel medic (Medicago truncatula) | |
| Pea (Pisum sativum) | http://pisum.bionet.nsc.ru/ |
| Cotton (Gossypium hirsutum) | http://probe.nalusda.gov:8300/plant/ |
| Soybean (Glycine max) | http://www.cbc.umn.edu/ResearchProjects/Soybean/index.html |
| Wheat (Triticum aestivum) | http://probe.nalusda gov:8300/plant/ |

Other plant databases can be accessed via http://www.hgmp.mrc.ac.uk/GenomeWeb/plant-gen-db.html

### EXAMPLE 3

Whole genome sequencing projects have been initiated for many organisms including two key plants, Arabidopsis thaliana and rice (Oryza sativa).

Arabidopsis thaliana is a small flowering plant that is widely used by plant science researchers as a model organism to study many aspects of plant biology. While Arabidopsis is not of major agronomic significance it does have several important advantages for the researchers in many areas of plant biology—especially genetics and molecular biology. These include:

A small genome size, which is distributed among five chromosomes;

Extensive genetic and physical maps

A rapid life cycle (about 6 weeks from seed to seed);

Easy cultivation in restricted space and prolific seed production;

Closely related to many commercially important crops such as Brassica napus (oilseed rape).

Easy transformation methods;

A large number of mutant lines

Such advantages have led to Arabidopsis becoming the “model organism” for studies of the molecular genetics of flowering plants.

Rice is one of three cereals produced annually at worldwide levels of approximately half a billion tons. Unlike the other major cereals, more than 90% of rice is consumed by humans. Approximately half of the world's population derives a significant proportion of their calorific intake from rice consumption. Application of molecular techniques to rice improvement will help to achieve better yields and improve nutritional value. In addition, rice is a model organism for studying the genes of other commercially important cereals such as maize, wheat and barley.

### EXAMPLE 4

The complete genome of Arabidopsis thaliana which is 130 Mb in size was screened for intermolecular peptides using the method described in patent application No. GB 9927485.4, filed Nov. 19, 1999. The gene, database accession number, its predicted interacting peptides and their position within the coding sequence of the gene are shown in the attached sequence listing: SEQ ID Nos. [1-1094].

### EXAMPLE 5

### Derivation of ‘Daughter’ Sequences from Parent Sequences

For each pair of ‘frames’ of amino acids which are deemed a ‘hit’ by the algorithm the current invention includes derived pairs of composite ‘daughter’ sequences of shorter frame lengths which automatically fulfil the same ‘complementary’ relationship.

For example, there is a complementary frame of size 10 between genes (inter-molecular) GRF1 and ATPK1 of Arabidopsis Thaliana.:

| GENE1 | GENE2 | Sequence 1 | Location | Sequence 2 | Location | Score |
|---|---|---|---|---|---|---|
| GRF1 | ATPK1 | IGARRASWRI | 61-70 | DSPASSPSSD | 430-439 | 10 |

One embodiment of the invention covers the derivation of the following sequences at frame length of 5:

| GENE | GENE2 | Sequence 1 | Location | Sequence 2 | Location | Score |
|---|---|---|---|---|---|---|
| GRF1 | ATPK1 | IGARR | 61-65 | DSPAS | 430-434 | 5 |
| GRF1 | ATPK1 | GARRA | 62-66 | SPASS | 431-435 | 5 |
| GRF1 | ATPK1 | ARRAS | 63-67 | PASSP | 432-436 | 5 |
| GRF1 | ATPK1 | RRASW | 64-68 | ASSPS | 433-437 | 5 |
| GRF1 | ATPK1 | RASWR | 65-69 | SSPSS | 434-438 | 5 |
| GRF1 | ATPK1 | ASWRI | 66-70 | SPSSD | 435-439 | 5 |

One embodiment of the invention covers the derivation of the following sequences at frame length of 6:

| GENE | GENE2 | Sequence 1 | Location | Sequence 2 | Location | Score |
|---|---|---|---|---|---|---|
| GRF1 | ATPK1 | IGARRA | 61-66 | DSPASS | 430-435 | 6 |
| GRF1 | ATPK1 | GARRAS | 62-67 | SPASSP | 431-436 | 6 |
| GRF1 | ATPK1 | ARRASW | 63-68 | PASSPS | 432-437 | 6 |
| GRF1 | ATPK1 | RRASWR | 64-69 | ASSPSS | 433-438 | 6 |
| GRF1 | ATPK1 | RASWRI | 65-70 | SSPSSD | 434-439 | 6 |

One embodiment of the invention covers the derivation of the following sequences at frame length of 7:

| GENE | GENE2 | Sequence 1 | Location | Sequence 2 | Location | Score |
|---|---|---|---|---|---|---|
| GRF1 | ATPK1 | IGARRAS | 61-67 | DSPASSP | 430-436 | 7 |
| GRF1 | ATPK1 | GARRASW | 62-68 | SPASSPS | 431-437 | 7 |
| GRF1 | ATPK1 | ARRASWR | 63-69 | PASSPSS | 432-438 | 7 |
| GRF1 | ATPK1 | RRASWRI | 64-70 | ASSPSSD | 433-439 | 7 |

One embodiment of the invention covers the derivation of the following sequences at frame length of 8:

| GENE | GENE2 | Sequence 1 | Location | Sequence 2 | Location | Score |
|---|---|---|---|---|---|---|
| GRF1 | ATPK1 | IGARRASW | 61-68 | DSPASSPS | 430-437 | 8 |
| GRF1 | ATPK1 | GARRASWR | 62-69 | SPASSPSS | 431-438 | 8 |
| GRF1 | ATPK1 | ARRASWRI | 63-70 | PASSPSSD | 432-439 | 8 |

One embodiment of the invention covers the derivation of the following sequences at frame length of 9:

| GENE | GENE2 | Sequence 1 | Location | Sequence 2 | Location | Score |
|---|---|---|---|---|---|---|
| GRF1 | ATPK1 | IGARRASWR | 61-69 | DSPASSPSS | 430-438 | 9 |
| GRF1 | ATPK1 | GARRASWRI | 62-70 | SPASSPSSD | 431-439 | 9 |

### EXAMPLE 6

The complete genome of Arabidopsis thaliana which is 130 Mb in size was screened for intramolecular peptides using the method described in patent application No. GB 9927485.4, filed Nov. 19, 1999. The gene, database accession number, peptide sequences and their position within the coding sequence of the gene are shown in the attached sequence listing: SEQ ID Nos. [1095-1143].

### EXAMPLE 7

### Derivation of ‘Daughter’ Sequences from Parent Sequences

For each pair of ‘frames’ of amino acids which are deemed a ‘hit’ by the algorithm the current invention includes derived pairs of composite ‘daughter’ sequences of shorter frame lengths which automatically fulfil the same ‘complementary’ relationship.

For example, gene ADH2 in Arabidopsis thaliana contains the following intra-molecular complementary relationship of frame length 10:

| GENE | Sequence 1 | Location | Sequence 2 | Location | Score |
|---|---|---|---|---|---|
| ADH2 | FGVNEFVNPK | 238-247 | FGVNEFVNPK | 238-247 | 10 |

One embodiment of the invention covers the derivation of the following sequences at frame length of 5:

| GENE | Sequence 1 | Location | Sequence 2 | Location | Score |
|---|---|---|---|---|---|
| ADH2 | FGVNE | 238-242 | KPNVF | 247-243 | 5 |
| ADH2 | GVNEF | 239-243 | PNVFE | 246-242 | 5 |
| ADH2 | VNEFV | 240-244 | NVFEN | 245-241 | 5 |
| ADH2 | NEFVN | 241-245 | VFENV | 244-240 | 5 |
| ADH2 | EFVNP | 242-246 | FENVG | 243-239 | 5 |
| ADH2 | FVNPK | 243-247 | ENVGF | 242-238 | 5 |

One embodiment of the invention covers the derivation of the following sequences at frame length of 6:

| GENE | Sequence 1 | Location | Sequence 2 | Location | Score |
|---|---|---|---|---|---|
| ADH2 | FGVNEF | 238-243 | KPNVFE | 247-242 | 6 |
| ADH2 | GVNEFV | 239-244 | PNVFEN | 246-241 | 6 |
| ADH2 | VNEFVN | 240-245 | NVFENV | 245-240 | 6 |
| ADH2 | NEFVNP | 241-246 | VFENVG | 244-239 | 6 |
| ADH2 | EFVNPK | 242-247 | FENVGF | 243-238 | 6 |

One embodiment of the invention covers the derivation of the following sequences at frame length of 7:

| GENE | Sequence 1 | Location | Sequence 2 | Location | Score |
|---|---|---|---|---|---|
| ADH2 | FGVNEFV | 238-244 | KPNVFEN | 247-241 | 7 |
| ADH2 | GVNEFVN | 239-245 | PNVFENV | 246-240 | 7 |
| ADH2 | VNEFVNP | 240-246 | NVFENVG | 245-239 | 7 |
| ADH2 | NEFVNPK | 241-247 | VFENVGF | 244-238 | 7 |

One embodiment of the invention covers the derivation of the following sequences at frame length of 8:

| GENE | Sequence 1 | Location | Sequence 2 | Location | Score |
|---|---|---|---|---|---|
| ADH2 | FGVNEFVN | 238-245 | KPNVFENV | 247-240 | 8 |
| ADH2 | GVNEFVNP | 239-246 | PNVFENVG | 246-239 | 8 |
| ADH2 | VNEFVNPK | 240-247 | NVFENVGF | 245-238 | 8 |

One embodiment of the invention covers the derivation of the following sequences at frame length of 9:

| GENE | Sequence 1 | Location | Sequence 2 | Location | Score |
|---|---|---|---|---|---|
| ADH2 | FGVNEFVNP | 238-245 | KPNVFENVG | 247-239 | 9 |
| ADH2 | GVNEFVNPK | 239-246 | PNVFENVGF | 246-238 | 9 |

### EXAMPLE 8

The genomes of the following plants were screened for intermolecular peptides in the same way as in Example 4.

Genome
Arabidopsis thaliana
Oilseed rape (Brassica napus)
Alfalfa (Medicago sativa)
Rice (Oryza sativa)
Sorghum (Sorghum bicolor)
Maize (Zea mays)
Loblolly Pine (Pinus taeda L.)
Barley (Hordeum vulgare)
Pearl Millet (Pennisetum glaucum)
Finger millet (Eleusine coracana)
Foxtail millet (Setaria italica)
Forage grasses, (Lolium Perenne,
Lolium multiflorum and Festuca
Pratensis)
Lotus japonicus (a model legume)
Barrel medic (Medicago truncatula)
Pea (Pisum sativum)
Cotton (Gossypium hirsutum)
Soybean (Glycine max)
Wheat (Triticum aestivum)

### EXAMPLE 9

The genomes of the following plants were screened for intermolecular peptides in the same way as in Example 6.

Genome
Arabidopsis thaliana
Oilseed rape (Brassica napus)
Alfalfa (Medicago sativa)
Rice (Oryza sativa)
Sorghum (Sorghum bicolor)
Maize (Zea mays)
Loblolly Pine (Pinus taeda L.)
Barley (Hordeum vulgare)
Pearl Millet (Pennisetum glaucum)
Finger millet (Eleusine coracana)
Foxtail millet (Setaria italica)
Forage grasses, (Lolium Perenne,
Lolium multiflorum and Festuca
Pratensis)
Lotus japonicus (a model legume)
Barrel medic (Medicago truncatula)
Pea (Pisum sativum)
Cotton (Gossypium hirsutum)
Soybean (Glycine max)
Wheat (Triticum aestivum)

### EXAMPLE 10

THE AMINO ACID PAIRINGS RESULTING FROM READING THE ANTICODON FOR
NATURALLY OCCURING AMINO ACID RESIDUES IN THE 5′-3′ DIRECTION

| Amino | | Complementary | Complementary | Amino | | Complementary | Complementary |
|---|---|---|---|---|---|---|---|
| Acid | codon | codon | Amino acid | Acid | codon | codon | Amino acid |
| Alanine | GCA | UGC | Cysteine | Serine | UCA | UGA | Stop |
| | GCG | CGC | Arginine | | UCC | GGA | Glycine |
| | GCC | GGC | Glycine | | UCG | CGA | Arginine |
| | GCU | AGC | Serine | | UCU | AGA | Arginine |
| | | | | | AGC | GCU | Alanine |
| | | | | | AGU | ACU | Threonine |
| Arginine | CGG | CCG | Proline | Glutamine | CAA | UUG | Leucine |
| | CGA | UCG | Serine | | CAG | CUG | Leucine |
| | CGC | GCG | Alanine | | | | |
| | CGU | ACG | Threonine | | | | |
| | AGG | CCU | Proline | | | | |
| | AGA | UCU | Serine | | | | |
| Aspartic Acid | GAC | GUC | Valine | Glycine | GGA | UCC | Serine |
| | GAU | AUC | Isoleucine | | GGC | GCC | Alanine |
| | | | | | GGU | ACC | Threonine |
| | | | | | GGG | CCC | Proline |
| Asparagine | AAC | GUU | Valine | Histidine | CAC | GUG | Valine |
| | AAU | AUU | Isoleucine | | CAU | AUG | Methionine |
| Cysteine | UGU | ACA | Threonine | Isoleucine | AUA | UAU | Tyrosine |
| | UGC | GCA | Alanine | | AUC | GAU | Aspartic |
| | | | | | AUU | AAU | acid |
| | | | | | | | Asparagine |
| Glutamic Acid | GAA | UUC | Phenylalanine | Leucine | CUG | CAG | Glutamine |
| | GAG | CUC | Leucine | | CUC | GAG | Glutamic |
| | | | | | CUU | AAG | acid |
| | | | | | UUA | UAA | Lysine |
| | | | | | CUA | UAG | Stop |
| | | | | | UUG | CAA | Stop |
| | | | | | CUG | CAG | Glutamine |
| | | | | | | | Glutamine |
| Lysine | AAA | UUU | Phenylalanine | Threonine | ACA | UGU | Cysteine |
| | AAG | CUU | Leucine | | ACG | CGU | Arginine |
| | | | | | ACC | GGU | Glycine |
| | | | | | ACU | AGU | Serine |
| Methionine | AUG | CAU | Histidine | Tryptophan | UGG | CCA | Proline |
| Phenylalanine | UUU | AAA | Lysine | Tyrosine | UAC | GUA | Valine |
| | UUC | GAA | Glutamic Acid | | UAU | AUA | Isoleucine |
| Proline | CCA | UGG | Tryptophan | Valine | GUA | UAC | Tyrosine |
| | CCC | GGG | Glycine | | GUG | CAC | Histidine |
| | CCU | AGG | Arginine | | GUC | GAC | Aspartic |
| | CCG | CGG | Arginine | | GUU | AAC | Acid |
| | | | | | | | Asparagine |

### EXAMPLE 11

The relationships between amino acids and the residues encoded in the complementary strand reading 3′-5′

| Amino | | Complementary | Complementary | Amino | | Complementary | CompleMentary |
|---|---|---|---|---|---|---|---|
| Acid | codon | codon | Amino acid | Acid | codon | codon | Amino acid |
| Alanine | GCA | CGU | Arginine | Serine | UCA | AGU | Serine |
| | GCG | CGC | | | UCC | AGG | Arginine |
| | GCC | CGG | | | UCG | AGC | Serine |
| | GCU | CGA | | | UCU | AGA | Arginine |
| | | | | | AGC | UCG | Serine |
| | | | | | AGU | UCA | Serine |
| Arginine | CGG | GCC | Alanine | Glutamine | CAA | GUU | Valine |
| | CGA | GCU | Alanine | | CAG | GUC | Valine |
| | CGC | GCG | Alanine | | | | |
| | CGU | GCA | Alanine | | | | |
| | AGG | UCC | Serine | | | | |
| | AGA | UCU | Serine | | | | |
| Aspartic | GAC | GUC | Valine | Glycine | GGA | CCU | Proline |
| Acid | GAU | AUC | Isoleucine | | GGC | CCG | Proline |
| | | | | | GGU | CCA | Proline |
| | | | | | GGG | CCC | Proline |
| Asparagine | AAC | UUG | Leucine | Histidine | CAC | GUG | Valine |
| | AAU | UUA | Leucine | | CAU | GUA | Valine |
| Cysteine | UGU | ACA | Threonine | Isoleucine | AUA | UAU | Tyrosine |
| | UGC | ACG | Threonine | | AUC | UAG | Stop |
| | | | | | AUU | UAA | Stop |
| Glutamic | GAA | CUU | Leucine | Leucine | CUG | GAC | Asp |
| Acid | GAG | CUG | Leucine | | CUC | GAG | Glutamic |
| | | | | | CUU | GAA | acid |
| | | | | | UUA | AAU | Glutamic |
| | | | | | CUA | GAU | Acid |
| | | | | | UUG | AAC | Asparagine |
| | | | | | CUG | GAC | Aspartic |
| | | | | | | | Acid |
| | | | | | | | Asparagine |
| | | | | | | | Aspartic |
| | | | | | | | Acid |
| Lysine | AAA | UUU | Phenylalanine | Threonine | ACA | UGU | Cysteine |
| | AAG | UUC | Phenylalanine | | ACG | UGC | Cysteine |
| | | | | | ACC | UGG | Tryptophan |
| | | | | | ACU | UGA | Stop |
| Methionine | AUG | UAC | Tyrosine | Tryptophan | UGG | ACC | Threonine |
| Phenylalanine | UUU | AAA | Lysine | Tyrosine | UAC | AUG | Methionine |
| | UUC | AAG | Lysine | | UAU | AUA | Isoleucine |
| Proline | CCA | GGU | Glycine | Valine | GUA | CAU | Histidine |
| | CCC | GGG | Glycine | | GUG | CAC | Histidine |
| | CCU | GGA | Glycine | | GUC | CAG | Glutamine |
| | CCG | GGC | Glycine | | GUU | CAA | Glutamine |

### REFERENCES

All publications, patents, and patent applications cited are hereby incorporated by reference in their entirety.

Baranyi L, Campbell W, Ohshima K, Fujimoto S, Boros M and Okada H. 1995. The antisense homology box: a new motif within proteins that encodes biologically active peptides. Nature Medicine. 1:894-901.

Gaasterland T. 1998. Structural genomics: Bioinformatics in the driver's seat. Nature Biotechnology 16: 645-627.

Goldstein D J. 1998. An unacknowledged problem for structural genomics? Nature Biotechnology 16: 696-697.

Sansom C. 1998. Extending the boundaries of molecular modelling. Nature Biotechnology 16: 917-918.

Stryer L. Biochmistry. 4th Edition. Freeman and Company, New York 1997.

We claim:

1. A set of peptide ligands; said set consisting of specific complementary peptides to proteins encoded by genes of plant genomes.

2. A set of peptide ligands according to claim 1, wherein the sequences of the peptides in the set are intra-molecular complementary peptide sequences.

3. A set of peptide ligands according to claim 1, wherein the sequences of the peptides in the set are inter-molecular complementary peptide sequences.

4. A novel peptide having a sequence which is a member of a set according to any preceding claim, capable of antagonising or agonising a specific interaction of a protein with another protein or receptor.

5. Use of a set of peptides according to any of claims 1 to 3 in an assay for screening and identification of one or more peptides according to claim 4.

6. Use according to claim 5 wherein the identified peptide(s) is a pesticide.

7. Use according to claim 5 wherein the identified peptide(s) is a herbicide.

8. A partly or wholly non-peptide mimetic of a peptide according to claim 4, 6 or 7, identified by use of the set of peptides according to claim 5.

9. A method for processing sequence data comprising the steps of:
selecting a first protein sequence and a second protein sequence;
selecting a frame size corresponding to a number of sequence elements such as amino acids or triplet codons, a score threshold, and a frame existence probability threshold;
comparing each frame of the first sequence with each frame of the second sequence by comparing pairs of sequence elements at corresponding positions within each such pair of frames to evaluate a complementary relationship score for each pair of frames;
storing details of any pairs of frames for which the score equals or exceeds the score threshold;
evaluating for each stored pair of frames the probability of the existence of that complementary pair of frames existing, on the basis of the number of possible complementary sequence elements existing for each sequence element in the pair of frames; and
discarding any stored pairs of frames for which the evaluated probability is greater than the probability threshold; wherein each frame is a peptide sequence of defined length.

10. A method according to claim 9, in which the first sequence is identical to the second sequence and a frame at a given position in the first sequence is only compared with frames in the second sequence at the same given position or at later positions in the second sequence, in order to eliminate repetition of comparisons.

11. A method according to claim 9 or 10, in which the sequence elements at corresponding positions within each of a pair of frames are compared sequentially, each such pair of sequence elements generating a score which is added to an aggregate score for the pair of frames.

12. A method according to claim 11, in which if the aggregate score reaches the score threshold before all the pairs of sequence elements in the pair of frames have been compared, details of the pair of frames are immediately stored and a new pair of frames is selected for comparison.

13. A method according to any preceding claim, in which the sequence elements are amino acids and pairs of amino acids are compared by using an antisense score list.

14. A method according to any of claims 9 to 12, in which the sequence elements are triplet codons and pairs of codons in corresponding positions within each it of the pairs of triplet codons are compared by using an antisense score list.

15. A method for processing sequence data substantially as described herein with reference to FIGS. 1 to 6.

16. A pair of frames or a list of pairs of frames being the product of the method of any of claims 9 to 15, optionally carried on a computer-readable medium.

17. A frame being the product of the method of any of claims 9 to 15, optionally carried on a computer-readable medium.

18. A peptide, pair of complementary peptides, or set of peptides, being the peptide(s) having the sequence of the frame(s) of claims 16 or 17.

19. A method for identifying a peptide pesticide or herbicide candidate, which method includes the steps of (i) identifying a set of specific complementary peptides according to any of claims 1 to 4; (ii) screening the set for specific protein interaction activity; and (iii) identifying one or more peptide(s) according to claim 5.
